(19) United States
(12) Patent Application Publication
(10) Pub. No.: US 2022/0098318 A1
(43) Pub. Date: Mar. 31, 2022

(54) DOSING FOR TREATMENT WITH ANTI-TIGIT AND ANTI-CD20 OR ANTI-CD38 ANTIBODIES

(71) Applicant: Genentech, Inc., South San Francisco, CA (US)

(72) Inventors: Raymond D. MENG, Mountain View, CA (US); Robert Wenchen HSIEH, Milbrae, CA (US); Namrata Srivastava PATIL, Sunnyvale, CA (US); Sean Keith KELLEY, Burlingame, CA (US); Karen Lynn KLING-MILLER, Livermore, CA (US); William Michael FLANAGAN, Menlo Park, CA (US)

(21) Appl. No.: 17/411,638

(22) Filed: Aug. 25, 2021

Related U.S. Application Data
(63) Continuation of application No. PCT/US2020/020135, filed on Feb. 27, 2020.
(60) Provisional application No. 62/866,309, filed on Jun. 25, 2019.
(60) Provisional application No. 62/835,941, filed on Apr. 18, 2019.
(60) Provisional application No. 62/832,769, filed on Apr. 11, 2019.
(60) Provisional application No. 62/811,513, filed on Feb. 27, 2019.

(51) Int. Cl. C07K 16/28; A61P 35/00

(52) U.S. Cl. CPC C07K 16/2896; A61K 2039/545; C07K 16/2887; A61P 35/00

(57) ABSTRACT

The invention provides methods of dosing for the treatment of cancers. In particular, provided are methods for treating human patients having cancer, such as a hematologic cancer, by administering a combination of an anti-TIGIT antagonist antibody and an anti-CD20 antibody or anti-CD38 antibody.

### SEQUENCE LISTING

The instant application contains a Sequence Listing which has been submitted electronically in ASCII format and is hereby incorporated by reference in its entirety. Said ASCII copy, created on Aug. 20, 2021, is named 50474-195006_Sequence_Listing_8_20_2021_ST25 and is 39,801 bytes in size.

### FIELD OF THE INVENTION

The present invention relates to the treatment of hematologic cancers. More specifically, the invention concerns the treatment of patients having a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL))) by administering a combination of an anti-T-cell immunoreceptor with Ig and ITIM domains (TIGIT) antagonist antibody and an anti-CD20 antibody or an anti-CD38 antibody.

### BACKGROUND OF THE INVENTION

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care, including the treatment of hematologic cancers such as lymphomas and myelomas, thus represent a significant and ever-increasing societal burden.

The most common hematologic cancer in adults is non-Hodgkin's lymphoma (NHL). Diffuse large B-cell lymphoma (DLBCL) is the most common aggressive NHL, accounting for approximately 30% of all NHLs diagnosed annually, and follicular lymphoma (FL) is the most common subtype of indolent NHL (iNHL), which accounts for approximately one-third of all NHLs. Nearly 40% of patients with DLBCL will eventually die of relapsed disease or disease that is refractory to first-line treatment, and FL remains an incurable disease with the currently available therapies. Another hematologic cancer, multiple myeloma (MM), affects almost 20,000 people every year in the United States, and worldwide, approximately 160,000 people are diagnosed with MM annually. MM remains incurable despite advances in treatment, with an estimated median survival of 8-10 years for standard-risk myeloma and 2-3 years for high-risk disease.

Thus, there is an unmet need in the field for the development of efficacious immunotherapies and methods of dosing the same which achieve a more favorable benefit-risk profile for the treatment of hematologic cancers, such as myelomas (e.g., MM) and lymphomas (e.g., NHL, e.g., DLBCL or FL).

### SUMMARY OF THE INVENTION

The present invention relates to methods of treating a subject having a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL))) by administering an anti-TIGIT antagonist antibody (e.g., an anti-TIGIT antagonist antibody as disclosed herein, e.g., tiragolumab) in combination with an anti-CD38 antibody (e.g., daratumumab) or an anti-CD20 antibody (e.g., rituximab).

In a first aspect, the disclosure features a method of treating a subject having a hematologic cancer comprising administering to the subject an anti-TIGIT antagonist antibody at a fixed dose of between about 30 mg to about 1200 mg and an anti-CD38 antibody at a dose of between about 8 mg/kg to about 24 mg/kg in a dosing regimen comprising at least nine dosing cycles, wherein (a) the anti-TIGIT antagonist antibody is administered once every three weeks, and (b) the anti-CD38 antibody is administered once every week during each of dosing cycles 1-3, once every three weeks during each of dosing cycles 4-8, and once every four weeks beginning on dosing cycle 9.

In some aspects, the length of each dosing cycle is 21 days. In some aspects, the anti-TIGIT antagonist antibody is administered on or about day 1 of each dosing cycle. In some aspects, the anti-CD38 antibody is administered on or about days 1, 8, and 15 of each of dosing cycles 1-3, on or about day 1 of each of dosing cycles 4-8, and on or about day 1 dosing cycle 9. In other aspects, the anti-TIGIT antagonist antibody and the anti-CD38 antibody are both administered on or about day 1 of each of dosing cycles 1-9.

In some aspects, the anti-TIGIT antagonist antibody is administered prior to the anti-CD38 antibody. In some aspects, the method comprises a first observation period following administration of the anti-TIGIT antagonist antibody and a second observation period following administration of the anti-CD38 antibody. In some aspects, the first observation period and the second observation period are each between about 30 minutes to about 60 minutes in length.

In some aspects, the anti-CD38 antibody is administered prior to the anti-TIGIT antagonist antibody. In some aspects the method comprises a first observation period following administration of the anti-CD38 antibody and a second observation period following administration of the anti-TIGIT antagonist antibody. In some aspects the first observation period and the second observation period are each between about 30 minutes to about 60 minutes in length.

In some aspects, the dosing regimen comprises at least 12 dosing cycles. In some aspects, the dosing regimen comprises at least 16 cycles.

In some aspects, the method further comprises administering to the subject a corticosteroid prior to each administration of the anti-CD38 antibody. In some aspects, the method further comprises administering to the subject an antipyretic prior to each administration of the anti-CD38 antibody. In some aspects, the method further comprises administering to the subject an antihistamine prior to each administration of the anti-CD38 antibody. In some aspects, the method further comprises administering to the subject a corticosteroid, an antipyretic, and an antihistamine prior to each administration of the anti-CD38 antibody. In some aspects, the corticosteroid is methylprednisolone, the antipyretic is acetaminophen, and the antihistamine is diphenhydramine. In some aspects, the method comprises administering to the subject a corticosteroid on each of the two days following administration of the anti-CD38 antibody.

In some aspects, the method comprises administering to the subject the anti-CD38 antibody at a dose of about 16 mg/kg.

In some aspects the anti-CD38 antibody is an anti-CD38 antagonist antibody. In some aspects, the anti-CD38 antibody comprises the following complementarity determining regions (CDRs): (a) a CDR-H1 comprising the amino acid sequence of SFAMS (SEQ ID NO: 20); (b) a CDR-H2 comprising the amino acid sequence of AISGSGGGTYYADSVKG (SEQ ID NO: 21); (c) a CDR-H3 comprising the amino acid sequence of DKILWFGEPVFDY (SEQ ID NO: 22); (d) a CDR-L1 comprising the amino acid sequence of RASQSVSSYLA (SEQ ID NO: 23); (e) a CDR-L2 comprising the amino acid sequence of DASNRAT (SEQ ID NO: 24); and (f) a CDR-L3 comprising the amino acid sequence of QQRSNWPPTF (SEQ ID NO: 25). In some aspects, the anti-CD38 antibody further comprises the following light chain variable region framework regions (FRs): (a) an FR-L1 comprising the amino acid sequence of EIVLTQSPATLSLSPGERATLSC (SEQ ID NO: 26); (b) an FR-L2 comprising the amino acid sequence of WYQQKPGQAPRLLIY (SEQ ID NO: 27); (c) an FR-L3 comprising the amino acid sequence of GIPARFSGSGSGTDFTLTISSLEPEDFAVYYC (SEQ ID NO: 28); and (d) an FR-L4 comprising the amino acid sequence of GQGTKVEIK (SEQ ID NO: 29). In some aspects, the anti-CD38 antibody further comprises the following heavy chain variable region FRs: (a) an FR-H1 comprising the amino acid sequence of EVQLLESGGGLVQPGGSLRLSCAVSGFTFN (SEQ ID NO: 30); (b) an FR-H2 comprising the amino acid sequence of WVRQAPGKGLEWVS (SEQ ID NO: 31); (c) an FR-H3 comprising the amino acid sequence of RFTISRDNSKNTLYLQMNSLRAEDTAVYFCAK (SEQ ID NO: 32); and (d) an FR-H4 comprising the amino acid sequence of WGQGTLVTVSS (SEQ ID NO: 33).

In some aspects, the anti-CD38 antibody further comprises: (a) a heavy chain variable (VH) domain comprising an amino acid sequence having at least 95% sequence identity to the amino acid sequence of EVQLLESGGGLVQPGGSLRLSCAVSGFTFNSFAMSWVRQAPGKGLEWVSAISGSGGGT YYADSVKGRFTISRDNSKNTLYLQMNSLRAEDTAVYFCAKDKILWFGEPVFDYWGQGTLVTVSS (SEQ ID NO: 34); (b) a light chain variable (VL) domain comprising an amino acid sequence having at least 95% sequence identity to the amino acid sequence of EIVLTQSPATLSLSPGERATLSCRASQSVSSYLAWYQQKPGQAPRLLIYDASNRATGIP ARFSGSGSGTDFTLTISSLEPEDFAVYYCQQRSNWPPTFGQGTKVEIK (SEQ ID NO: 35); or (c) a VH domain as in (a) and a VL domain as in (b).

In some aspects, the anti-CD38 antibody comprises: (a) a VH domain comprising the amino acid sequence of SEQ ID NO: 34; and (b) a VL domain comprising the amino acid sequence of SEQ ID NO: 35.

In some aspects, the anti-CD38 antibody is a monoclonal antibody. In some aspects, the anti-CD38 antibody is a human antibody. In some aspects, the anti-CD38 antibody is a full-length antibody. In some aspects, the anti-CD38 antibody is daratumumab.

In some aspects, the anti-CD38 antibody is an antibody fragment that binds CD38 selected from the group consisting of Fab, Fab′, Fab′-SH, Fv, single chain variable fragment (scFv), and (Fab′)₂ fragments.

In some aspects, the anti-CD38 antibody is an IgG class antibody. In some aspects, the IgG class antibody is an IgG1 subclass antibody.

In some aspects, the method comprises administering to the subject the anti-CD38 antibody intravenously.

In some aspects, the hematologic cancer is a myeloma. In some aspects, the myeloma is a multiple myeloma (MM). In some aspects, the MM is a relapsed or refractory MM.

In another aspect, the disclosure features a method for treating a subject having a hematologic cancer, the method comprising administering to the subject an anti-TIGIT antagonist antibody at a fixed dose of between about 30 mg to about 1200 mg and an anti-CD20 antibody at a dose of between about 250 mg/m² to about 500 mg/m² in a dosing regimen comprising at least a first, a second, and a third dosing cycle, wherein: (a) the anti-TIGIT antagonist antibody is administered once every three weeks; and (b) the anti-CD20 antibody is administered once every week.

In some aspects, (a) each dosing cycle of the dosing regimen comprises a single dose of the anti-TIGIT antagonist antibody; (b) the first dosing cycle comprises a first dose (C1D1), a second dose (C1D2), and a third dose (C1D3) of the anti-CD20 antibody; (c) the second dosing cycle comprises a first dose (C2D1), a second dose (C2D2), and a third dose (C2D3) of the anti-CD20 antibody; and (d) the third dosing cycle comprises at least a first dose (C3D1) and a second dose (C3D2) of the anti-CD20 antibody. In some aspects, the dosing regimen comprises a total of eight doses of the anti-CD20 antibody. In some aspects, the length of each dosing cycle is 21 days.

In some aspects, the method comprises administering to the subject the anti-TIGIT antagonist antibody on or about day 1 of each dosing cycle. In some aspects, the method comprises administering to the subject the C1 D1, the C1 D2, and the C1 D3 of the anti-CD20 antibody on or about days 1, 8, and 15, respectively, of the first dosing cycle. In some aspects, the method comprises administering to the subject the C2D1, the C2D2, and the C2D3 of the anti-CD20 antibody on or about days 1, 8, and 15, respectively, of the second dosing cycle. In some aspects, the method comprises administering to the subject the C3D1 and the C3D2 of the anti-CD20 antibody on or about days 1 and 8, respectively, of the third dosing cycle. In some aspects, the anti-TIGIT antagonist antibody and the anti-CD20 antibody are both administered on or about day 1 of each of dosing cycles 1, 2, and 3.

In some aspects, the anti-TIGIT antagonist antibody is administered prior to the anti-CD20 antibody. In some aspects, the method comprises a first observation period following administration of the anti-TIGIT antagonist antibody and a second observation period following administration of the anti-CD20 antibody. In some aspects, the first observation period and the second observation period are each between about 30 minutes to about 60 minutes in length.

In some aspects, the dosing regimen comprises at least 12 dosing cycles. In some aspects, the dosing regimen comprises at least 16 cycles.

In some aspects, the subject has an infusion-related reaction (IRR) to the anti-TIGIT antagonist antibody, and the method further comprises administering to the subject an antihistamine and/or an antipyretic prior to a subsequent administration of the anti-TIGIT antagonist antibody.

In some aspects, the method further comprises administering to the subject an antipyretic and an antihistamine prior to each administration of the anti-CD20 antibody. In some aspects the antipyretic is acetaminophen and the antihistamine is diphenhydramine. In some aspects, the method further comprises administering to the subject a glucocorticoid prior to each administration of the anti-CD20 antibody.

In some aspects, the method comprises administering to the subject the anti-CD20 antibody at a dose of about 375 mg/m².

In some aspects, the anti-CD20 antibody is an anti-CD20 antagonist antibody. In some aspects, the anti-CD20 antibody comprises the following CDRs: (a) a CDR-H1 comprising the amino acid sequence of SYNMH (SEQ ID NO: 36); (b) a CDR-H2 comprising the amino acid sequence of AIYPGNGDTSYNQKFKG (SEQ ID NO: 37); (c) a CDR-H3 comprising the amino acid sequence of STYYGGDWYFNV (SEQ ID NO: 38); (d) a CDR-L1 comprising the amino acid sequence of RASSSVSYIH (SEQ ID NO: 39); (e) a CDR-L2 comprising the amino acid sequence of ATSNLAS (SEQ ID NO: 40); and (f) a CDR-L3 comprising the amino acid sequence of QQWTSNPPT (SEQ ID NO: 41). In some aspects, the anti-CD20 antibody further comprises the following light chain variable region FRs: (a) an FR-L1 comprising the amino acid sequence of QIVLSQSPAILSASPGEKVTMTC (SEQ ID NO: 42); (b) an FR-L2 comprising the amino acid sequence of WFQQKPGSSPKPWIY (SEQ ID NO: 43); (c) an FR-L3 comprising the amino acid sequence of GVPVRFSGSGSGTSYSLTISRVEAEDAATYYC (SEQ ID NO: 44); and (d) an FR-L4 comprising the amino acid sequence of FGGGTKLEIK (SEQ ID NO: 45). In some aspects, the anti-CD20 antibody further comprises the following heavy chain variable region FRs: (a) an FR-H1 comprising the amino acid sequence of QVQLQQPGAELVKPGASVKMSCKASGYTFT (SEQ ID NO: 46); (b) an FR-H2 comprising the amino acid sequence of WVKQTPGRGLEWIG (SEQ ID NO: 47); (c) an FR-H3 comprising the amino acid sequence of KATLTADKSSSTAYMQLSSLTSEDSAVYYCAR (SEQ ID NO: 48); and (d) an FR-H4 comprising the amino acid sequence of WGAGTTVTVS (SEQ ID NO: 49).

In some aspects, the anti-CD20 antibody further comprises: (a) a VH domain comprising an amino acid sequence having at least 95% sequence identity to the amino acid sequence of QVQLQQPGAELVKPGASVKMSCKASGYTFTSYNMHWVKQTPGRGLEWIGAIYPGNGDTSYNQKFK GKATLTADKSSSTAYMQLSSLTSEDSAVYYCARSTYYGGDWYFNVWGAGTTVTVS (SEQ ID NO: 50); (b) a VL domain comprising an amino acid sequence having at least 95% sequence identity to the amino acid sequence of QIVLSQSPAILSASPGEKVTMTCRASSSVSYIHWFQQKPGSSPKPWIYATSNLASGVPVRFSGSGSG TSYSLTISRVEAEDAATYYCQQWTSNPPTFGGGTKLEIK (SEQ ID NO: 51); or (c) a VH domain as in (a) and a VL domain as in (b).

In some aspects, the anti-CD20 antibody comprises: (a) a VH domain comprising the amino acid sequence of SEQ ID NO:50; and (b) a VL domain comprising the amino acid sequence of SEQ ID NO:51.

In some aspects, the anti-CD20 antibody is a monoclonal antibody. In some aspects, the anti-CD20 antibody is a chimeric antibody. In some aspects, the anti-CD20 antibody is a full-length antibody. In some aspects, the anti-CD20 antibody is rituximab.

In some aspects, the anti-CD20 antibody is an antibody fragment that binds CD20 selected from the group consisting of Fab, Fab′, Fab′-SH, Fv, single chain variable fragment (scFv), and (Fab′)₂ fragments.

In some aspects, the anti-CD20 antibody is an IgG class antibody. In some aspects, the IgG class antibody is an IgG1 subclass antibody.

In some aspects, the method comprises administering to the subject the anti-CD20 antibody intravenously.

In some aspects, the hematologic cancer is a lymphoma. In some aspects, the lymphoma is a non-Hodgkin's lymphoma (NHL). In some aspects, the NHL is a relapsed or refractory diffuse large B cell lymphoma (DLBCL). In some aspects, the NHL is a relapsed or refractory follicular lymphoma (FL).

In some aspects, the method comprises administering to the subject an anti-TIGIT antagonist antibody at a fixed dose of between about 30 mg to about 600 mg. In some aspects, the method comprises administering to the subject an anti-TIGIT antagonist antibody at a fixed dose of about 600 mg.

In some aspects, the anti-TIGIT antagonist antibody comprises the following CDRs: (a) a CDR-H1 comprising the amino acid sequence of SNSAAWN (SEQ ID NO: 1); (b) a CDR-H2 comprising the amino acid sequence of KTYYRFKWYSDYAVSVKG (SEQ ID NO: 2); (c) a CDR-H3 comprising the amino acid sequence of ESTTYDLLAGPFDY (SEQ ID NO: 3); (d) a CDR-L1 comprising the amino acid sequence of KSSQTVLYSSNNKKYLA (SEQ ID NO: 4); (e) a CDR-L2 comprising the amino acid sequence of WASTRES (SEQ ID NO: 5); and (f) a CDR-L3 comprising the amino acid sequence of QQYYSTPFT (SEQ ID NO: 6). In some aspects, the anti-TIGIT antagonist antibody further comprises the following light chain variable region FRs: (a) an FR-L1 comprising the amino acid sequence of DIVMTQSPDSLAVSLGERATINC (SEQ ID NO: 7); (b) an FR-L2 comprising the amino acid sequence of WYQQKPGQPPNLLIY (SEQ ID NO: 8); (c) an FR-L3 comprising the amino acid sequence of GVPDRFSGSGSGTDFTLTISSLQAEDVAVYYC (SEQ ID NO: 9); and (d) an FR-L4 comprising the amino acid sequence of FGPGTKVEIK (SEQ ID NO: 10). In some aspects, the anti-TIGIT antagonist antibody further comprises the following heavy chain variable region FRs: (a) an FR-H1 comprising the amino acid sequence of X₁VQLQQSGPGLVKPSQTLSLTCAISGDSVS (SEQ ID NO: 11), wherein X₁ is Q or E; (b) an FR-H2 comprising the amino acid sequence of WIRQSPSRGLEWLG (SEQ ID NO: 12); (c) an FR-H3 comprising the amino acid sequence of RITINPDTSKNQFSLQLNSVTPEDTAVFYCTR (SEQ ID NO: 13); and (d) an FR-H4 comprising the amino acid sequence of WGQGTLVTVSS (SEQ ID NO: 14). In some aspects, X₁ is Q. In some aspects, X₁ is E.

In some aspects, the anti-TIGIT antagonist antibody comprises: (a) a VH domain comprising an amino acid sequence having at least 95% sequence identity to the amino acid sequence of EVQLQQSGPGLVKPSQTLSLTCAISGDSVSSNSAAWNWIRQSPSRGLEWLGKTYYRFKWYSDYAVS VKGRITINPDTSKNQFSLQLNSVTPEDTAVFYCTRESTTYDLLAGPFDYWGQGTLVTVSS (SEQ ID NO: 17) or QVQLQQSGPGLVKPSQTLSLTCAISGDSVSSNSAAWNWIRQSPSRGLEWLGKTYYRFKWYSDYAVS VKGRITINPDTSKNQFSLQLNSVTPEDTAVFYCTRESTTYDLLAGPFDYWGQGTLVTVSS (SEQ ID NO: 18); (b) a VL domain comprising an amino acid sequence having at least 95% sequence identity to the amino acid sequence of DIVMTQSPDSLAVSLGERATINCKSSQTVLYSSNNKKYLAWYQQKPGQPPNLLIYWASTRESGVPDR FSGSGSGTDFTLTISSLQAEDVAVYYCQQYYSTPFTFGPGTKVEIK (SEQ ID NO: 19); or (c) a VH domain as in (a) and a VL domain as in (b).

In some aspects, the anti-TIGIT antagonist antibody comprises: (a) a VH domain comprising the amino acid sequence of SEQ ID NO: 17 or 18; and (b) a VL domain comprising the amino acid sequence of SEQ ID NO: 19.

In some aspects, the anti-TIGIT antagonist antibody is a monoclonal antibody. In some aspects, the anti-TIGIT antagonist antibody is a human antibody. In some aspects, the anti-TIGIT antagonist antibody is a full-length antibody. In some aspects, the anti-TIGIT antagonist antibody is tiragolumab.

In some aspects, the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

In some aspects, the anti-TIGIT antagonist antibody is an IgG class antibody. In some aspects, the IgG class antibody is an IgG1 subclass antibody.

In some aspects, the method comprises administering to the subject the anti-TIGIT antagonist antibody intravenously.

In another aspect, the disclosure provides a method for treating a subject having a relapsed or refractory MM, the method comprising administering to the subject tiragolumab at a fixed dose of 600 mg and daratumumab at a dose of 16 mg/kg in a dosing regimen comprising at least nine dosing cycles, wherein the length of each dosing cycle is 21 days, and wherein: (a) tiragolumab is administered on or about day 1 of each dosing cycle; and (b) daratumumab is administered on or about days 1, 8, and 15 of each of dosing cycles 1-3, on or about day 1 during each of dosing cycles 4-8, and once every 4 weeks beginning on or about day 1 of dosing cycle 9.

In another aspect, the disclosure provides a method of treating a subject having a relapsed or refractory NHL, the method comprising administering to the subject tiragolumab at a fixed dose of 600 mg and rituximab at a dose of 375 mg/m² in a dosing regimen comprising at least a first, a second, and a third dosing cycle, wherein the length of each dosing cycle is 21 days, and wherein: (a) each dosing cycle comprises a single dose of tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; (c) the second dosing cycle further comprises a first dose (C2D1), a second dose (C2D2), and a third dose (C2D3) of rituximab administered on or about days 1, 8, and 15, respectively of the second dosing cycle; and (d) the third dosing cycle further comprises a first dose (C3D1) and a second dose (C3D2) of rituximab, wherein the C3D1 and the C3D2 are administered on or about days 1 and 8, respectively, of the third dosing cycle, and wherein the dosing regimen comprises a total of eight doses of rituximab.

In some aspects, the dosing regimen comprises at least 12 dosing cycles. In some aspects, the dosing regimen comprises at least 16 dosing cycles.

In another aspect, the disclosure provides a kit comprising an anti-TIGIT antagonist antibody, an anti-CD38 antibody, and a package insert comprising instructions to administer the anti-TIGIT antagonist antibody and the anti-CD38 antibody to a subject having a hematologic cancer in accordance with any of the methods disclosed herein. In some aspects, the anti-TIGIT antagonist antibody is tigarolumab and the anti-CD38 antibody is daratumumab.

In another aspect, the disclosure provides a kit comprising an anti-TIGIT antagonist antibody, an anti-CD38 antibody or an anti-CD20 antibody, and a package insert comprising instructions to administer the anti-TIGIT antagonist antibody and the anti-CD38 antibody or anti-CD20 antibody to a subject having a hematologic cancer in accordance with any the methods disclosed herein. In some aspects, the anti-TIGIT antagonist antibody is tiragolumab and the anti-CD20 antibody is rituximab.

In another aspect, the disclosure provides a method for treating a subject having a relapsed or refractory MM, the method comprising administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle. In some aspects, tiragolumab is administered as a monotherapy.

In another aspect, the disclosure provides a method for treating a subject having a relapsed or refractory NHL, the method comprising administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle. In some aspects, tiragolumab is administered as a monotherapy.

In some aspects, the method comprises an observation period following administration of tiragolumab. In some aspects, the observation period is between about 30 minutes to about 60 minutes in length.

In some aspects, the dosing regimen comprises at least 12 dosing cycles. In some aspects, the dosing regimen comprises at least 16 dosing cycles.

In some aspects, the subject has an infusion-related reaction (IRR) to tiragolumab, and the method further comprises administering to the subject an antihistamine and/or an antipyretic prior to a subsequent administration of tiragolumab.

In some aspects, the method comprises administering to the subject tiragolumab intravenously.

### BRIEF DESCRIPTION OF THE DRAWINGS

FIG. 1 is a diagram outlining the dosing schedule of a combination therapy with an anti-CD38 antibody (e.g., daratumumab) and anti-TIGIT antagonist antibody (e.g., tiragolumab).

### DETAILED DESCRIPTION OF THE INVENTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The “amount,” “level,” or “expression level,” used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker can be used to identify/characterize a subject having a cancer (e.g., a hematologic cancer (e.g., a myeloma (e.g., MM, e.g., a relapsed or refractory MM) or a lymphoma (e.g., a NHL, e.g., a relapsed or refractory DLBCL or a relapsed or refractory FL))) who may be likely to respond to, or benefit from, a particular therapy (e.g., a therapy comprising one or more dosing cycles of an anti-TIGIT antagonist antibody and an anti-CD38 or an anti-CD20 antibody).

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 52), as well as any form of TIGIT that results from processing in the cell (e.g., processed human TIGIT without a signal sequence, having the amino acid sequence of SEQ ID NO: 53). The term also encompasses naturally occurring variants of TIGIT, e.g., splice variants or allelic variants. The amino acid sequence of an exemplary human TIGIT may be found under UniProt Accession Number Q495A1.

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to substantially or completely restore a functional response by T-cells (e.g., proliferation, cytokine production, target cell killing) from a dysfunctional state to antigen stimulation. It will be understood by one of ordinary skill in the art that in some aspects, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one aspect, the extent of binding of an anti-TIGIT antagonist antibody to an unrelated, non-TIGIT protein is less than about 10% of the binding of the antibody to TIGIT as measured, e.g., by a radioimmunoassay (RIA). In certain aspects, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-TIGIT antagonist antibody binds to an epitope of TIGIT that is conserved among TIGIT from different species or an epitope on TIGIT that allows for cross-species reactivity.

“CD20” and “CD20 antigen” are used interchangeably herein and refer to a transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells, or normal plasma cells. CD20 is present on both normal B cells as well as malignant B cells, and is expressed in >90% of B cell NHLs. CD20 includes any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, e.g., splice variants or allelic variants. Other names for CD20 in the literature include “B-lymphocyte-restricted differentiation antigen” and “Bp35”. The CD20 antigen is encoded by the MS4A1 gene. The nucleic acid sequence of an exemplary human MS4A1 is shown under NCBI Reference Sequence: NM_152866.2 or in SEQ ID NO: 54. The amino acid sequence of an exemplary CD20 protein encoded by MS4A1 is shown under UniProt Accession No. P11836 or in SEQ ID NO: 55. The CD20 antigen is described in, for example, Clark and Ledbetter, Adv. Can. Res. 52:81-149 (1989) and Valentine et al. J. Biol. Chem. 264(19):11282-11287 (1989).

“Anti-CD20 antibody” and “CD20 binding antibody” are used interchangeably herein and encompass all antibodies that bind CD20 with sufficient affinity such that the antibody is useful as a therapeutic agent in targeting a cell expressing the antigen, and do not significantly cross-react with other proteins such as a negative control protein in the assays described below. For example, an anti-CD20 antibody may bind to CD20 on the surface of a malignant B cell and mediate B cell lysis through the activation of complement-dependent lysis, antibody-dependent cellular cytotoxicity (ADCC), and apoptosis mediated by Fc cross-linking, leading to the depletion of circulating B lymphocytes. In certain aspects, an anti-CD20 antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, an anti-CD20 antibody that binds to CD20 has a K_{D} of <10 nM. In certain aspects, the binding is at a K_{D} of <7.5 nM, <5 nM, between 1-5 nM, or <1 nM. In certain aspects, the anti-CD20 antibody may bind to both human CD20 and cyno CD20. Anti-CD20 antibodies also include anti-CD20 antagonist antibodies. Bispecific antibodies wherein one arm of the antibody binds CD20 are also contemplated. Also encompassed by this definition of anti-CD20 antibody are functional fragments of the preceding antibodies.

Examples of antibodies which bind the CD20 antigen include: “C2B8” which is now called “rituximab” (“RITUXAN®”) (U.S. Pat. No. 5,736,137, expressly incorporated herein by reference); the yttrium-[90]-labeled 2B8 murine antibody designated “Y2B8” or “Ibritumomab Tiuxetan” ZEVALIN® (U.S. Pat. No. 5,736,137, expressly incorporated herein by reference); murine IgG2a “B11,” also called “tositumomab,” (Beckman Coulter) optionally labeled with ¹³¹I to generate the “131I-B1” antibody (iodine 1131 tositumomab, BEXXAR™) (U.S. Pat. No. 5,595,721, expressly incorporated herein by reference); murine monoclonal antibody “1F” (Press et al. Blood 69(2):584-591 (1987) and variants thereof including “framework patched” or humanized 1F5 (WO03/002607, Leung, S.); ATCC deposit HB-96450); murine 2H7 and chimeric 2H7 antibody (U.S. Pat. No. 5,677,180, expressly incorporated herein by reference); humanized 2H7; huMax-CD20 or “ofatumumab” ARZERRA® (Genmab, Denmark); AME-133 (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)).

The terms “rituximab” or “RITUXAN®” herein refer to the genetically engineered chimeric murine/human monoclonal antibody directed against the CD20 antigen and designated “C2B8” in U.S. Pat. No. 5,736,137, expressly incorporated herein by reference, including fragments thereof which retain the ability to bind CD20.

“CD38” as used herein, refers to a CD38 glycoprotein found on the surface of many immune cells, including CD4+, CD8+, B lymphocytes, and natural killer (NK) cells, and includes any native CD38 from any vertebrate source, including mammals such as primates (e.g., humans) and rodents (e.g., mice and rats), unless otherwise indicated. CD38 is expressed at a higher level and more uniformly on myeloma cells as compared to normal lymphoid and myeloid cells. The term encompasses “full-length,” unprocessed CD38, as well as any form of CD38 that results from processing in the cell. The term also encompasses naturally occurring variants of CD38, e.g., splice variants or allelic variants. CD38 is also referred to in the art as cluster of differentiation 38, ADP-ribosyl cyclase 1, cADPr hydrolase 1, and cyclic ADP-ribose hydrolase 1. CD38 is encoded by the CD38 gene. The nucleic acid sequence of an exemplary human CD38 is shown under NCBI Reference Sequence: NM_001775.4 or in SEQ ID NO: 56. The amino acid sequence of an exemplary human CD38 protein encoded by CD38 is shown under UniProt Accession No. P28907 or in SEQ ID NO: 57.

The term “anti-CD38 antibody” encompass all antibodies that bind CD38 with sufficient affinity such that the antibody is useful as a therapeutic agent in targeting a cell expressing the antigen, and do not significantly cross-react with other proteins such as a negative control protein in the assays described below. For example, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. An anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the anti-CD38 antibody may bind to both human CD38 and chimpanzee CD38. Anti-CD38 antibodies also include anti-CD38 antagonist antibodies. Bispecific antibodies wherein one arm of the antibody binds CD38 are also contemplated. Also encompassed by this definition of anti-CD38 antibody are functional fragments of the preceding antibodies. Examples of antibodies which bind CD38 include: daratumumab (DARZALEX®) (U.S. Pat. No. 7,829,673 and U.S. Pub. No: 20160067205 A1, expressly incorporated herein by reference); “MOR202” (U.S. Pat. No. 8,263,746, expressly incorporated herein by reference); and isatuximab (SAR-650984) (U.S. Pat. No. 8,153,765, expressly incorporated herein by reference).

As used herein, “administering” is meant a method of giving a dosage of a compound (e.g., an anti-TIGIT antibody, an anti-CD20 antibody, or an anti-CD38 antibody) or a composition (e.g., a pharmaceutical composition, e.g., a pharmaceutical composition including an anti-TIGIT antibody, anti-CD20 antibody, and/or anti-CD38 antibody) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

A “fixed” or “flat” dose of a therapeutic agent (e.g., an anti-TIGIT antagonist antibody) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in combination with” or “in conjunction with” refers to administration of one treatment modality in addition to another treatment modality. As such, “in combination with” or “in conjunction with” refers to administration of one treatment modality before, during, or after administration of the other treatment modality to the individual.

A “disorder” or “disease” is any condition that would benefit from treatment including, but not limited to, disorders that are associated with some degree of abnormal cell proliferation, e.g., cancer, e.g., a hematologic cancer, e.g., a myeloma (e.g., multiple myeloma (MM), e.g., a relapsed or refractory MM) or a lymphoma (e.g., a NHL, e.g., a relapsed or refractory diffuse large B cell lymphoma (DLBCL) or a relapsed or refractory follicular lymphoma (FL))).

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include, but are not limited to, hematologic cancers including myeloma and B cell lymphoma (including MM (e.g., relapsed or refractory MM), DLBCL (e.g., relapsed or refractory DLBCL), FL (e.g., relapsed or refractory FL), low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); lung cancer, such as non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer); esophageal cancer; cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer; bladder cancer (e.g., urothelial bladder cancer (UBC), muscle invasive bladder cancer (MIBC), and BCG-refractory non-muscle invasive bladder cancer (NMIBC)); cancer of the urinary tract; hepatoma; breast cancer (e.g., HER2+ breast cancer and triple-negative breast cancer (TNBC), which are estrogen receptors (ER−), progesterone receptors (PR−), and HER2 (HER2−) negative); colon cancer; rectal cancer; colorectal cancer; endometrial or uterine carcinoma; salivary gland carcinoma; kidney or renal cancer (e.g., renal cell carcinoma (RCC)); prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)). Examples of anti-cancer therapeutic agents include, but are limited to, e.g., immunomodulatory agents (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), an anti-TIGIT antagonist antibody, or an anti-PD-L1 antagonist antibody, or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody)), chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/32 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, an anti-TIGIT antagonist antibody, an anti-CD20 antibody, or an anti-CD38 antibody, or a composition (e.g., pharmaceutical composition) thereof, is at least the minimum amount required to achieve the desired therapeutic result, such as a measurable increase in overall survival or progression-free survival of a particular disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, increasing the quality of life of those suffering from the disease, decreasing the dose of other medications required to treat the disease, enhancing effect of another medication such as via targeting, delaying the progression of the disease (e.g. progression-free survival); delay of unequivocal clinical progression (e.g., cancer-related pain progression, deterioration in Eastern Cooperative Group Oncology Group (ECOG) Performance Status (PS) (e.g., how the disease affects the daily living abilities of the patient), and/or initiation of next systemic anti-cancer therapy), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with a TIGIT and/or anti-CD20 or anti-CD38 antibody (e.g., anti-TIGIT antagonist antibodies and/or anti-CD20 antibodies or anti-CD38 antibodies).

“Individual response” or “response” can be assessed using any endpoint indicating a benefit to the subject, including, without limitation, (1) inhibition, to some extent, of disease progression (e.g., progression of cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)); (6) increase or extend in the length of survival, including overall survival and progression-free survival; and/or (9) decreased mortality at a given point of time following treatment.

An “objective response” refers to a measurable response including complete response (CR) or partial response (PR). In some aspects, “objective response rate” (ORR) refers to the sum of complete response (CR) rate and partial response (PR) rate. For MM, ORR may be defined as the proportion of patients with best overall response of stringent complete response (sCR), complete response (CR), very good partial response (VGPR), or partial response (PR) (see, e.g., Table 2, below), as defined by the International Myeloma Working Group Uniform Response (IMWG) criteria, as disclosed in Durie et al. Leukemia. 20(9):1467-73 (2006), Durie et al. Leukemia. 29:2416-7 (2015), and Kumar et al. Lancet Oncol. 17:e328-46 (2016), which are incorporated herein by reference in their entireties. For NHL, ORR may be defined as the proportion of patients with a CR or PR on two consecutive occasions 4 weeks apart, according to the Lugano Response Criteria for Malignant Lymphoma (Lugano) classification (see, e.g., Table 4, below), as described in Cheson et al. J. Clin. Oncol. 32(27):3059-3067 (2014), which is incorporated herein by reference in its entirety.

As used herein, “duration of objective response” (DOR) is defined as the time from the first occurrence of a documented objective response to disease progression (e.g., according to IMWG criteria for MM (see, e.g., Tables 2 and 3, below) or according to the Lugano classification for NHL (see, e.g., Table 4, below)), or death from any cause within 30 days of the last dose of a treatment, whichever occurs first.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment. In some aspects, OS may be defined as the time from enrollment to death from any cause.

As used herein, “progression-free survival” (PFS) refers to the length of time during and after treatment during which the disease being treated (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)) does not get worse, i.e., does not progress (e.g., according to IMWG criteria for MM (see, e.g., Tables 2 and 3, below) or according to the Lugano classification for NHL (see, e.g., Table 4, below). Progression-free survival may include the amount of time patients have experienced a complete response or a partial response, as well as the amount of time patients have experienced stable disease. As the skilled person will appreciate, a patients' progression-free survival is improved or enhanced if the patient experiences a longer length of time during which the disease does not progress as compared to the average or mean progression-free survival time of a control group of similarly situated patients.

As used herein, “complete response” or “CR” refers to disappearance of all signs of cancer (e.g., disappearance of target lesions). This does not always mean the cancer has been cured. For MM, CR is further defined according to the IMWG criteria (e.g., as described in Table 2, below). For NHL, CR is further defined according to the Lugano classification (e.g., as described in Table 4, below).

As used herein, “stringent complete response” or “sCR” refers to a complete response as defined by the IMWG criteria (e.g., as described in Table 2, below) plus normal free light chain (FLC) ratio and absence of clonal cells in bone marrow by immunohistochemistry (kappa/lambda ratio ≤4:1 or ≥1:2 for kappa and lambda patients, respectively after counting ≥100 plasma cells).

As used herein, “partial response” or “PR,” refers to a decrease in the size of one or more lesions or tumors, or in the extent of cancer in the body, in response to treatment. With respect to MM, PR refers to at least a 50% reduction of serum M-protein and at least a 90% reduction in 24 hr urinary M-protein or to a level of less than 200 mg/24 hr. For MM, PR is further defined according to the IMWG criteria (e.g., as described in Table 2, below). Partial response, with respect to NHL, refers to at least a 50% decrease in the sum of the product of the perpendicular diameters for multiple lesions (SPD) of up to six target measurable nodes and extranodal sites; a score of 4 or 5 with reduced uptake compared to baseline and residual masses of the lymph nodes and extralymphatic sites; spleen enlargement regression of at least 50% in length beyond normal; residual uptake of higher than normal bone marrow, but reduced compared with baseline; a non-measured lesion that is absent, normal, or regressed (i.e., that has not increased); and/or an absence of new lesions. For NHL, PR is further defined according to the Lugano classification (e.g., as described in Table 4, below).

As used herein, “very good partial response” or “VGPR” refers to serum and urine M-protein detectable by immunofixation but not on electrophoresis; or ≥90% reduction in serum M-protein-plus urine M-protein level <100 mg/24 hr, as defined by the IMGW criteria (see, e.g., Table 2, below).

As used herein, “minimal response” or “MR” is defined per the IMGW criteria (see, e.g., Table 3, below) and refers to ≥25% but ≤49% reductions of serum M-protein and reduction in 24-hour urine M-protein by 50%-89%, and additionally, if present at baseline, 25%-49% reduction in the size (SPD) ° of soft tissue plasmacytomas.

As used herein, “stable disease” or “SD” refers to neither sufficient shrinkage of target lesions and/or a decrease in the extent of cancer in the body to qualify for PR, nor sufficient increase to qualify for PD. For MM, SD refers to a response otherwise not meeting the criteria for MR, CR, VGPR, PR, or PD as defined according to the IMWG criteria (e.g., as described in Tables 2 and 3, below). SD, with respect to NHL, refers to (a) less than a 50% decrease from baseline in SPD of up to 6 dominant, measurable nodes and extranodal sites, without meeting criteria for progressive disease, (b) a score of 4 or 5 with no significant change in fluorodeoxyglucose (FDG) uptake from baseline at interim or end of treatment in the target nodes/nodal masses, and/or extranodal lesions, (c) no change from baseline for the bone marrow, (d) the absence of increases consistent with progression in the non-measured lesion or with respect to organ enlargement, and/or (e) the absence of the formation of new lesions. SD for NHL is further defined according to the Lugano classification (e.g., as described in Table 4, below).

As used herein, “progressive disease” or “PD” refers to an increase in the size of one or more lesions or tumors, or in the extent of cancer in the body, in response to treatment. PD, with respect to MM, refers to an increase of at least 25% from the lowest response value in at least one of the following: (a) serum M-protein, (b) urine M-protein, (c) the difference between involved and uninvolved FLC levels, (d) bone marrow plasma cell percentage irrespective of baseline status, (e) the appearance of new lesion(s), or (f) at least a 50% increase in circulating plasma cells. For MM, PD is further defined according to the IMWG criteria (e.g., as described in Table 3, below). For NHL, PD refers to one or more of (a) cross product of the longest transverse diameter of a lesion (LDi) and perpendicular diameter (PPD) progression, (b) abnormalities of the individual target nodes/nodal masses or extranodal lesions, (c) a score of 4 or 5 with an increase in the intensity of uptake from baseline, (d) new FDG-avid foci, (e) new or recurrent splenomegaly, (f) new or clear progression of preexisting non-measured lesions), (g) regrowth of previously resolved lesions, (h) a new node, extranodal site, or assessable disease of any size attributable to lymphoma (e.g., new FDG-avid foci consistent with lymphoma), and (i) new or recurrent FDG avid-foci or new or recurrent involvement of the bone marrow. For NHL, PD is further defined according to the Lugano classification (e.g., as described in Table 4, below).

“Clinical relapse,” as used herein refers to direct indications of increasing disease and/or end organ dysfunction relating to the underlying clonal plasma cell proliferative disorder. For MM, clinical relapse is defined according to the IMWG criterial (see, e.g., table 3, below) and includes one or more of (a) development of new soft tissue plasmacytomas or bone lesions, (b) definite increase in the size of existing plasmacytomas or bone lesions, defined as a 50% (and ≥1 cm) increase as measured serially by the sum of the products of the cross-diameters of the measurable lesion, (c) hypercalcemia >11 mg/dL (2.65 mm/L), (d) decrease in in hemoglobin of ≥2 g/dL (1.25 mmol/L) not related to therapy or other non-myeloma related conditions, (e) a rise in serum creatinine by 2 mg/dL or more (177 μmol/L or more) from the start of therapy and attributable to myeloma, and/or (f) hyperviscosity related to serum paraprotein.

As used herein, “delaying progression” of a disorder or disease means to defer, hinder, slow, retard, stabilize, and/or postpone development of the disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, development of central nervous system (CNS) metastasis, may be delayed.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)), the presence or size of metastases, or the size of the primary tumor.

By “extending survival” is meant increasing overall or progression-free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including stringent complete response (sCR), complete response (CR), very good partial response (VGPR), partial response (PR), and minimal response (MR).

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)) characterized by certain, molecular, pathological, histological, and/or clinical features. In some aspects, a biomarker is a gene. Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)₂, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:

(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);

(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., TIGIT or PD-L1). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one aspect, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some aspects, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred aspects, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Nat. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one aspect, the extent of binding of an antibody to an unrelated target is less than about 10% of the binding of the antibody to the target as measured, for example, by a radioimmunoassay (RIA). In certain aspects, an antibody that specifically binds to a target has a dissociation constant (K_{D}) of ≤1 μM, ≤100 nM, ≤10 nM, ≤1 nM, or ≤0.1 nM. In certain aspects, an antibody specifically binds to an epitope on a protein that is conserved among the protein from different species. In another aspect, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one aspect, the term “specific binding” refers to binding where a molecule binds to a particular polypeptide or epitope on a particular polypeptide without substantially binding to any other polypeptide or polypeptide epitope.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for aspect, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “subject” or “individual” is meant a mammal, including, but not limited to, a human or non-human mammal, such as a bovine, equine, canine, ovine, or feline. In some aspects, the subject is a human. Patients are also subjects herein.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample obtained from a subject of interest that would be expected or is known to contain the cellular and/or molecular entity that is to be characterized. In some aspects, the sample is a tumor tissue sample (e.g., a tumor biopsy, e.g., a lymph node biopsy (e.g., lymph fluid)), a bone marrow sample (e.g., a bone marrow aspirate), or a blood sample (e.g., a whole blood sample, a serum sample, or a plasma sample). Other samples include, but are not limited to, primary or cultured cells or cell lines, cell supernatants, cell lysates, platelets, vitreous fluid, synovial fluid, follicular fluid, seminal fluid, amniotic fluid, milk, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for aspect, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, aspects wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

An “article of manufacture” is any manufacture (e.g., a package or container) or kit comprising at least one reagent, e.g., a medicament for treatment of a disease or disorder (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)), and a package insert. In certain aspects, the manufacture or kit is promoted, distributed, or sold as a unit for performing the methods described herein.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments.

### III. Therapeutic Methods and Uses

Provided herein are methods and uses for treating cancer (e.g., a hematologic cancer, e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory FL)) in a subject comprising administering to the subject one or more dosing cycles of an effective amount of an anti-TIGIT antagonist antibody and anti-CD20 or an anti-CD38 antibody.

Dosing Regimens and Administration

The therapeutic methods and uses of the invention described herein, include, in one aspect, administering to a subject having a cancer (e.g., a hematologic cancer, e.g., a myeloma (e.g., a multiple myeloma (MM), e.g., a relapsed or refractory MM)) an effective amount of an anti-TIGIT antagonist antibody (e.g., an anti-TIGIT antagonist antibody described herein, e.g., tiragolumab) and an anti-CD38 antibody (e.g., dartumumab) in a dosing regimen comprising at least nine dosing cycles, wherein (a) the anti-TIGIT antagonist antibody is administered once every three weeks; and (b) the anti-CD38 antibody is administered once every week during each of dosing cycles 1-3, once every three weeks during each of dosing cycles 4-8, and once every four weeks beginning on dosing cycle 9, thereby treating the subject.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In some aspects, the effective amount of the anti-CD38 antibody (e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., daratumumab) is a dose of about 16 mg/kg.

In any of the methods and uses of the invention, the anti-TIGIT antagonist antibody (e.g., an anti-TIGIT antagonist antibody as disclosed herein, e.g., tiragolumab) and the anti-CD38 antibody (e.g., daratumumab) may be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-TIGIT antagonist antibody (e.g., an anti-TIGIT antagonist antibody as disclosed herein, e.g., tiragolumab) and the anti-CD38 antibody (e.g., daratumumab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-TIGIT antagonist antibody (e.g., an anti-TIGIT antagonist antibody as disclosed herein, e.g., tiragolumab) is administered on about day 1 (e.g., day 1±1 day) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab) is administered intravenously at a fixed dose of about 600 mg on day 2 of each 21 day cycle (i.e., at a fixed dose of about 600 mg every three weeks). Similarly, in some aspects, the anti-CD38 antibody (e.g., daratumumab) is administered on or about days 1 (e.g., day 1±1 day), 8 (e.g., day 8±1 day), and 15 (e.g., day 15±1 day) of each of dosing cycles 1-3, on or about day 1 (e.g., day 1±1 day) of each of dosing cycles 4-8, and on or about day 1 (e.g., day 1±1 day) of dosing cycle 9. For example, the anti-CD38 antibody is administered intravenously at a dose of 16 mg/kg on each of days 1, 8, and 15 of dosing cycles 1, 2, and 3; on day 1 of each of dosing cycles 4, 5, 6, 7, 8, and 9. In some aspects, the anti-CD38 antibody (e.g., daratumumab) is administered once every four weeks beginning on or about day 1 of cycle nine. For example, the anti-CD38 antibody (e.g., daratumumab) is administered intravenously at a dose of 16 mg/kg on day 1 of dosing cycle nine, on day 8 of dosing cycle 10, on day 15 of dosing cycle 11, on day 1 of dosing cycle 13, on day 8 of dosing cycle 14, on day 15 of dosing cycle 15, on day 1 of dosing cycle 17, and once every four weeks thereafter. In some aspects, any of the doses of the anti-CD38 antibody (e.g., daratumumab) may be split into two doses and administered to the subject over the course of two consecutive days. In some aspects, the first dose of the anti-CD38 antibody (e.g., daratumumab) is administered over days 1 and 2 of cycle 1.

In some aspects, when the anti-TIGIT antagonist antibody (e.g., an anti-TIGIT antagonist antibody as disclosed herein, e.g., tiragolumab) and the anti-CD38 antibody (e.g., daratumumab) are scheduled to be administered on the same day, the anti-CD38 antibody may be administered either on that day, or on the next consecutive day. Accordingly, in some aspects, the anti-TIGIT antagonist antibody (e.g., an anti-TIGIT antagonist antibody as disclosed herein, e.g., tiragolumab) is administered to the subject on day 1 of the dosing cycle and the anti-CD38 antibody (e.g. daratumumab) is administered to the subject on day 2 of the dosing cycle. In other aspects, the anti-TIGIT antagonist antibody (e.g., an anti-TIGIT antagonist antibody as disclosed herein, e.g., tiragolumab) and the anti-CD38 antibody (e.g. daratumumab) are both administered to the subject on day 1 of the dosing cycle. In aspects in which the anti-TIGIT antagonist antibody (e.g., an anti-TIGIT antagonist antibody as disclosed herein, e.g., tiragolumab) and the anti-CD38 antibody (e.g. daratumumab) are both administered to the subject on the same day, the anti-TIGIT antagonist antibody (e.g., an anti-TIGIT antagonist antibody as disclosed herein, e.g., tiragolumab) is administered before the anti-CD38 antibody (e.g. daratumumab).

In some aspects, the anti-TIGIT antagonist antibody (e.g., an anti-TIGIT antagonist antibody as disclosed herein, e.g., tiragolumab) is administered to the subject before the anti-CD38 antibody (e.g., daratumumab). In some aspects, for example, following administration of the anti-TIGIT antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-TIGIT antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-CD38 antibody during the first and second observation periods, respectively.

In other aspects, the anti-CD38 antibody (e.g. daratumumab) is administered to the subject before the anti-TIGIT antagonist antibody (e.g., an anti-TIGIT antagonist antibody as disclosed herein, e.g., tiragolumab). In some aspects, for example, following administration of the anti-CD38 antibody and before administration of the anti-TIGIT antagonist antibody, the method includes an intervening first observation period. In some aspects, the method includes a second observation period following administration of the anti-TIGIT antagonist antibody. In some aspects, the method includes both a first observation period following administration of the anti-CD38 antibody and second observation period following administration of the anti-TIGIT antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-TIGIT antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-TIGIT antagonist antibody during the first and second observation periods, respectively.

In some aspects, the methods and uses further include administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., daratumumab). For example, 100 mg IV methylprednisolone, 650-1000 mg oral acetaminophen, and/or 25-50 mg oral or IV diphenhydramine is administered to the subject about one to three hours prior to the administration of the anti-CD38 antibody. In other aspects, the methods and uses include administering to the subject a corticosteroid on each of the two days following administration of the anti-CD38 antibody (e.g., daratumumab), beginning on the day following administration. For example, 20 mg methylprednisolone is administered to the subject on days 1 and 2 following administration of the anti-CD38 antibody.

In another aspect, the invention provides a method of treating a subject having a relapsed or refractory MM by administering to the subject tiragolumab at a fixed dose of 600 mg and daratumumab at a dose of 16 mg/kg in a dosing regimen comprising at least nine dosing cycles, wherein the length of each dosing cycle is 21 days, and wherein: (a) tiragolumab is administered on or about day 1 of each dosing cycle; and (b) daratumumab is administered on or about days 1, 8, and 15 of each of dosing cycles 1-3, on or about day 1 during each of dosing cycles 4-8, and once every 4 weeks beginning on or about day 1 of dosing cycle 9. In some aspects, the dosing regimen comprises at least 12 dosing cycles. In other aspects, the dosing regimen comprises at least 16 dosing cycles.

In another aspect, the invention provides an anti-TIGIT antagonist antibody (e.g., an anti-TIGIT antagonist antibody disclosed herein, e.g., tiragolumab) and anti-CD38 antibody (e.g., daratumumab) for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of an anti-TIGIT antagonist antibody (e.g., an anti-TIGIT antagonist antibody described herein, e.g., tiragolumab) and an anti-CD38 antibody (e.g., dartumumab) in a dosing regimen comprising at least nine dosing cycles, wherein (a) the anti-TIGIT antagonist antibody is administered once every three weeks; and (b) the anti-CD38 antibody is administered once every week during each of dosing cycles 1-3, once every three weeks during each of dosing cycles 4-8, and once every four weeks beginning on dosing cycle 9.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In some aspects, the effective amount of the anti-CD38 antibody (e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., daratumumab) is a dose of about 16 mg/kg.

In any of the uses of the invention, the anti-TIGIT antagonist antibody (e.g., an anti-TIGIT antagonist antibody as disclosed herein, e.g., tiragolumab) and the anti-CD38 antibody (e.g., daratumumab) is to be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-TIGIT antagonist antibody (e.g., an anti-TIGIT antagonist antibody as disclosed herein, e.g., tiragolumab) and the anti-CD38 antibody (e.g., daratumumab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-TIGIT antagonist antibody (e.g., an anti-TIGIT antagonist antibody as disclosed herein, e.g., tiragolumab) is to be administered on about day 1 (e.g., day 1±1 day) of each dosing cycle. For example, the anti-TIGIT antagonist antibody (e.g., an anti-TIGIT antagonist antibody as disclosed herein, e.g., tiragolumab) is to be administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab) is to be administered intravenously at a fixed dose of about 600 mg on day 2 of each 21 day cycle (i.e., at a fixed dose of about 600 mg every three weeks). Similarly, in some aspects, the anti-CD38 antibody (e.g., daratumumab) is to be administered on or about days 1 (e.g., day 1±1 day), 8 (e.g., day 8±1 day), and 15 (e.g., day 15±1 day) of each of dosing cycles 1-3, on or about day 1 (e.g., day 1±1 day) of each of dosing cycles 4-8, and on or about day 1 (e.g., day 1±1 day) of dosing cycle 9. For example, the anti-CD38 antibody is to be administered intravenously at a dose of 16 mg/kg on each of days 1, 8, and 15 of dosing cycles 1, 2, and 3; on day 1 of each of dosing cycles 4, 5, 6, 7, 8, and 9. In some aspects, the anti-CD38 antibody (e.g., daratumumab) is to be administered once every four weeks beginning on or about day 1 of cycle nine. For example, the anti-CD38 antibody (e.g., daratumumab) is to be administered intravenously at a dose of 16 mg/kg on day 1 of dosing cycle nine, on day 8 of dosing cycle 10, on day 15 of dosing cycle 11, on day 1 of dosing cycle 13, on day 8 of dosing cycle 14, on day 15 of dosing cycle 15, on day 1 of dosing cycle 17, and once every four weeks thereafter. In some aspects, any of the doses of the anti-CD38 antibody (e.g., daratumumab) may be split into two doses and is to be administered to the subject over the course of two consecutive days. In some aspects, the first dose of the anti-CD38 antibody (e.g., daratumumab) is to be administered to the subject over days 1 and 2 of cycle 1.

In some aspects, when the anti-TIGIT antagonist antibody (e.g., an anti-TIGIT antagonist antibody as disclosed herein, e.g., tiragolumab) and the anti-CD38 antibody (e.g., daratumumab) are scheduled to be administered on the same day, the anti-CD38 antibody is to be administered either on that day, or on the next consecutive day. Accordingly, in some aspects, the anti-TIGIT antagonist antibody (e.g., an anti-TIGIT antagonist antibody as disclosed herein, e.g., tiragolumab) is to be administered to the subject on day 1 of the dosing cycle and the anti-CD38 antibody (e.g. daratumumab) is to be administered to the subject on day 2 of the dosing cycle. In other aspects, the anti-TIGIT antagonist antibody (e.g., an anti-TIGIT antagonist antibody as disclosed herein, e.g., tiragolumab) and the anti-CD38 antibody (e.g. daratumumab) are both to be administered to the subject on day 1 of the dosing cycle. In aspects in which the anti-TIGIT antagonist antibody (e.g., an anti-TIGIT antagonist antibody as disclosed herein, e.g., tiragolumab) and the anti-CD38 antibody (e.g. daratumumab) are both to be administered to the subject on the same day, the anti-TIGIT antagonist antibody (e.g., an anti-TIGIT antagonist antibody as disclosed herein, e.g., tiragolumab) is to be administered before the anti-CD38 antibody (e.g. daratumumab).

In some aspects, the anti-TIGIT antagonist antibody (e.g., an anti-TIGIT antagonist antibody as disclosed herein, e.g., tiragolumab) is to be administered to the subject before the anti-CD38 antibody (e.g., daratumumab). In some aspects, for example, following administration of the anti-TIGIT antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-TIGIT antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-CD38 antibody during the first and second observation periods, respectively.

In other aspects, the anti-CD38 antibody (e.g. daratumumab) is to be administered to the subject before the anti-TIGIT antagonist antibody (e.g., an anti-TIGIT antagonist antibody as disclosed herein, e.g., tiragolumab). In some aspects, for example, following administration of the anti-CD38 antibody and before administration of the anti-TIGIT antagonist antibody, the method includes an intervening first observation period. In some aspects, the method includes a second observation period following administration of the anti-TIGIT antagonist antibody. In some aspects, the method includes both a first observation period following administration of the anti-CD38 antibody and second observation period following administration of the anti-TIGIT antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and anti-TIGIT antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and anti-TIGIT antagonist antibody during the first and second observation periods, respectively.

In some aspects, the method further includes administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., daratumumab). In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., daratumumab). For example, 100 mg IV methylprednisolone, 650-1000 mg oral acetaminophen, and/or 25-50 mg oral or IV diphenhydramine is to be administered to the subject about one to three hours prior to the administration of the anti-CD38 antibody. In other aspects, the method includes administering to the subject a corticosteroid on each of the two days following administration of the anti-CD38 antibody (e.g., daratumumab), beginning on the day following administration. For example, 20 mg methylprednisolone is to be administered to the subject on days 1 and 2 following administration of the anti-CD38 antibody.

In another aspect, the invention provides tiragolumab and daratumumab for use in a method of treating a subject having a relapsed or refractory MM, wherein the method comprises administering to the subject tiragolumab at a fixed dose of 600 mg and daratumumab at a dose of 16 mg/kg in a dosing regimen comprising at least nine dosing cycles, wherein the length of each dosing cycle is 21 days, and wherein: (a) tiragolumab is administered on or about day 1 of each dosing cycle; and (b) daratumumab is administered on or about days 1, 8, and 15 of each of dosing cycles 1-3, on or about day 1 during each of dosing cycles 4-8, and once every 4 weeks beginning on or about day 1 of dosing cycle 9. In some aspects, the dosing regimen comprises at least 12 dosing cycles. In other aspects, the dosing regimen comprises at least 16 dosing cycles.

In another aspect, the invention provides uses of an effective amount of an anti-TIGIT antagonist antibody (e.g., an anti-TIGIT antagonist antibody disclosed herein, e.g., tiragolumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-TIGIT antagonist antibody in combination with an effective amount of an anti-CD38 antibody (e.g., daratumumab) in a dosing regimen comprising at least nine dosing cycles, wherein (a) the medicament comprising the anti-TIGIT antagonist antibody is administered once every three weeks; and (b) the anti-CD38 antibody is administered once every week during each of dosing cycles 1-3, once every three weeks during each of dosing cycles 4-8, and once every four weeks beginning on dosing cycle 9.

In another aspect, the invention provides uses of an effective amount of an anti-CD38 antibody (e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-CD38 antibody in combination with an effective amount of an anti-TIGIT antagonist antibody (e.g., an anti-TIGIT antagonist antibody disclosed herein, e.g., tiragolumab) in a dosing regimen comprising at least nine dosing cycles, wherein (a) the anti-TIGIT antagonist antibody is administered once every three weeks; and (b) the medicament comprising the anti-CD38 antibody is administered once every week during each of dosing cycles 1-3, once every three weeks during each of dosing cycles 4-8, and once every four weeks beginning on dosing cycle 9.

In another aspect, the invention provides uses of an effective amount of an anti-TIGIT antagonist antibody (e.g., an anti-TIGIT antagonist antibody disclosed herein, e.g., tiragolumab) and an effective amount of an anti-CD38 antibody (e.g., daratumumab) in the manufacture or preparation of a medicament for use in a method of treating a subject having a cancer (e.g., a hematologic cancer, e.g., a myeloma (e.g., a multiple myeloma (MM), e.g., a relapsed or refractory MM)), wherein the method comprises administering to the subject an effective amount of the medicament comprising the anti-TIGIT antagonist antibody in combination with an effective amount of a medicament comprising the anti-CD38 antibody in a dosing regimen comprising at least nine dosing cycles, wherein (a) the medicament comprising the anti-TIGIT antagonist antibody is administered once every three weeks; and (b) the medicament comprising the anti-CD38 antibody is administered once every week during each of dosing cycles 1-3, once every three weeks during each of dosing cycles 4-8, and once every four weeks beginning on dosing cycle 9.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In some aspects, the effective amount of the anti-CD38 antibody (e.g., daratumumab) is a dose of between about 8 mg/kg to about 24 mg/kg of the subject's body weight (e.g., between about 8 mg/kg to about 22 mg/kg, e.g., between about 10 mg/kg to about 20 mg/kg, e.g., between about 10 mg/kg to about 18 mg/kg, e.g., between about 12 mg/kg to about 16 mg/kg, e.g., about 16±2 mg/kg, about 16±1 mg/kg, about 16±0.5 mg/kg, about 16±0.2 mg/kg, or about 16±0.1 mg/kg, e.g., about 16 mg/kg). In some aspects, the effective amount of anti-CD38 antibody (e.g., daratumumab) is a dose of about 16 mg/kg.

In any of the uses of the invention, (a) the medicament comprising the anti-TIGIT antagonist antibody (e.g., an anti-TIGIT antagonist antibody as disclosed herein, e.g., tiragolumab) and the anti-CD38 antibody (e.g., daratumumab), (b) the medicament comprising and the anti-CD38 antibody (e.g., daratumumab) and the anti-TIGIT antagonist antibody (e.g., an anti-TIGIT antagonist antibody as disclosed herein, e.g., tiragolumab), or (c) the medicament comprising the anti-TIGIT antagonist antibody (e.g., an anti-TIGIT antagonist antibody as disclosed herein, e.g., tiragolumab) and the medicament comprising and the anti-CD38 antibody (e.g., daratumumab) are to be administered in a dosing regimen that includes at least nine dosing cycles (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of (a) the medicament comprising the anti-TIGIT antagonist antibody (e.g., an anti-TIGIT antagonist antibody as disclosed herein, e.g., tiragolumab) and the anti-CD38 antibody (e.g., daratumumab), (b) the medicament comprising and the anti-CD38 antibody (e.g., daratumumab) and the anti-TIGIT antagonist antibody (e.g., an anti-TIGIT antagonist antibody as disclosed herein, e.g., tiragolumab), or (c) the medicament comprising the anti-TIGIT antagonist antibody (e.g., an anti-TIGIT antagonist antibody as disclosed herein, e.g., tiragolumab) and the medicament comprising and the anti-CD38 antibody (e.g., daratumumab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-TIGIT antagonist antibody (e.g., an anti-TIGIT antagonist antibody as disclosed herein, e.g., tiragolumab), or medicament thereof, is to be administered on about day 1 (e.g., day 1±1 day) of each dosing cycle. For example, the anti-TIGIT antagonist antibody (e.g., an anti-TIGIT antagonist antibody as disclosed herein, e.g., tiragolumab), or medicament thereof, is to be administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab), or medicament thereof, is to be administered intravenously at a fixed dose of about 600 mg on day 2 of each 21 day cycle (i.e., at a fixed dose of about 600 mg every three weeks). Similarly, in some aspects, the anti-CD38 antibody (e.g., daratumumab), or medicament thereof, is to be administered on or about days 1 (e.g., day 1±1 day), 8 (e.g., day 8±1 day), and 15 (e.g., day 15±1 day) of each of dosing cycles 1-3, on or about day 1 (e.g., day 1±1 day) of each of dosing cycles 4-8, and on or about day 1 (e.g., day 1±1 day) of dosing cycle 9. For example, the anti-CD38 antibody (e.g., daratumumab), or medicament thereof, is to be administered intravenously at a dose of 16 mg/kg on each of days 1, 8, and 15 of dosing cycles 1, 2, and 3; on day 1 of each of dosing cycles 4, 5, 6, 7, and 8; and on day 9 of dosing cycle 9. In some aspects, the anti-CD38 antibody (e.g., daratumumab), or medicament thereof, is to be administered once every four weeks beginning on or about day 1 of cycle nine. For example, the anti-CD38 antibody (e.g., daratumumab), or medicament thereof, is to be administered intravenously at a dose of 16 mg/kg on day 1 of dosing cycle nine, on day 8 of dosing cycle 10, on day 15 of dosing cycle 11, on day 1 of dosing cycle 13, on day 8 of dosing cycle 14, on day 15 of dosing cycle 15, on day 1 of dosing cycle 17, and once every four weeks thereafter. In some aspects, any of the doses of the anti-CD38 antibody (e.g., daratumumab), or medicament thereof, may be split into two doses and administered to the subject over the course of two consecutive days. In some aspects, the first dose of the anti-CD38 antibody (e.g., daratumumab), or medicament thereof, is administered over days 1 and 2 of cycle 1.

In some aspects, when the anti-TIGIT antagonist antibody (e.g., an anti-TIGIT antagonist antibody as disclosed herein, e.g., tiragolumab), or medicament thereof, and the anti-CD38 antibody (e.g., daratumumab), or medicament thereof, are scheduled to be administered on the same day, the anti-CD38 antibody, or medicament thereof, is to be administered either on that day, or on the next consecutive day. Accordingly, in some aspects, the anti-TIGIT antagonist antibody (e.g., an anti-TIGIT antagonist antibody as disclosed herein, e.g., tiragolumab), or medicament thereof, is to be administered to the subject on day 1 of the dosing cycle and the anti-CD38 antibody (e.g. daratumumab), or medicament thereof, is to be administered to the subject on day 2 of the dosing cycle. In other aspects, the anti-TIGIT antagonist antibody (e.g., an anti-TIGIT antagonist antibody as disclosed herein, e.g., tiragolumab), or medicament thereof, and the anti-CD38 antibody (e.g. daratumumab), or medicament thereof, are both to be administered to the subject on day 1 of the dosing cycle. In aspects in which the anti-TIGIT antagonist antibody (e.g., an anti-TIGIT antagonist antibody as disclosed herein, e.g., tiragolumab), or medicament thereof, and the anti-CD38 antibody (e.g. daratumumab), or medicament thereof, are both to be administered to the subject on the same day, the anti-TIGIT antagonist antibody (e.g., an anti-TIGIT antagonist antibody as disclosed herein, e.g., tiragolumab), or medicament thereof, is to be administered before the anti-CD38 antibody (e.g. daratumumab), or medicament thereof.

In some aspects, the anti-TIGIT antagonist antibody (e.g., an anti-TIGIT antagonist antibody as disclosed herein, e.g., tiragolumab) is to be administered to the subject before the anti-CD38 antibody (e.g., daratumumab). In some aspects, for example, following administration of the anti-TIGIT antagonist antibody and before administration of the anti-CD38 antibody, the method includes an intervening first observation period. In some aspects, the method further includes a second observation period following administration of the anti-CD38 antibody. In some aspects, the method includes both a first observation period following administration of the anti-TIGIT antagonist antibody and second observation period following administration of the anti-CD38 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-CD38 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-CD38 antibody during the first and second observation periods, respectively.

In other aspects, the anti-CD38 antibody (e.g. daratumumab) is to be administered to the subject before the anti-TIGIT antagonist antibody (e.g., an anti-TIGIT antagonist antibody as disclosed herein, e.g., tiragolumab). In some aspects, for example, following administration of the anti-CD38 antibody and before administration of the anti-TIGIT antagonist antibody, the method includes an intervening first observation period. In some aspects, the method includes a second observation period following administration of the anti-TIGIT antagonist antibody. In some aspects, the method includes both a first observation period following administration of the anti-CD38 antibody and second observation period following administration of the anti-TIGIT antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD38 antibody and the anti-TIGIT antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD38 antibody and the anti-TIGIT antagonist antibody during the first and second observation periods, respectively.

In some aspects, the method further includes administering to the subject one or more of a corticosteroid (e.g., methylprednisolone), an antipyretic (e.g., acetaminophen), and an antihistamine (e.g., diphenhydramine) prior to each administration of the anti-CD38 antibody (e.g., daratumumab), or medicament thereof. In some aspects, the methods and uses further include administering to the subject a corticosteroid (e.g., methylprednisolone), an antipyretic (e.g., acetaminophen), and an antihistamine (e.g., diphenhydramine) prior to each administration of the anti-CD38 antibody (e.g., daratumumab), or medicament thereof. For example, 100 mg IV methylprednisolone, 650-1000 mg oral acetaminophen, and/or 25-50 mg oral or IV diphenhydramine is to be administered to the subject about one to three hours prior to the administration of the anti-CD38 antibody, or medicament thereof. In other aspects, the method includes administering to the subject a corticosteroid on each of the two days following administration of the medicament comprising the anti-CD38 antibody (e.g., daratumumab), or medicament thereof, beginning on the day following administration. For example, 20 mg methylprednisolone is to be administered to the subject on days 1 and 2 following administration of the anti-CD38 antibody, or medicament thereof.

In another aspect, the invention provides tiragolumab in the manufacture or preparation of a medicament for use in a method of treating a subject having relapsed or refractory MM, wherein the method comprises administering to the subject 600 mg of the medicament comprising the anti-TIGIT antagonist antibody in combination with 16 mg/kg of daratumumab in a dosing regimen comprising at least nine dosing cycles, wherein (a) the medicament comprising the anti-TIGIT antagonist antibody is administered once every three weeks; and (b) daratumumab is administered once every week during each of dosing cycles 1-3, once every three weeks during each of dosing cycles 4-8, and once every four weeks beginning on dosing cycle 9. In some aspects, the dosing regimen comprises at least 12 dosing cycles. In other aspects, the dosing regimen comprises at least 16 dosing cycles.

In another aspect, the invention provides daratumumab in the manufacture or preparation of a medicament for use in a method of treating a subject having relapsed or refractory MM, wherein the method comprises administering to the subject 600 mg of tiragolumab in combination with 16 mg/kg of the medicament comprising daratumumab in a dosing regimen comprising at least nine dosing cycles, wherein (a) tiragolumab is administered once every three weeks; and (b) the medicament comprising daratumumab is administered once every week during each of dosing cycles 1-3, once every three weeks during each of dosing cycles 4-8, and once every four weeks beginning on dosing cycle 9. In some aspects, the dosing regimen comprises at least 12 dosing cycles. In other aspects, the dosing regimen comprises at least 16 dosing cycles.

In another aspect, the invention provides tiragolumab and daratumumab in the manufacture or preparation of a medicament for use in a method of treating a subject having relapsed or refractory MM, wherein the method comprises administering to the subject 600 mg of the medicament comprising the anti-TIGIT antagonist antibody in combination with 16 mg/kg of the medicament comprising daratumumab in a dosing regimen comprising at least nine dosing cycles, wherein (a) the medicament comprising the anti-TIGIT antagonist antibody is administered once every three weeks; and (b) the medicament comprising daratumumab is administered once every week during each of dosing cycles 1-3, once every three weeks during each of dosing cycles 4-8, and once every four weeks beginning on dosing cycle 9. In some aspects, the dosing regimen comprises at least 12 dosing cycles. In other aspects, the dosing regimen comprises at least 16 dosing cycles.

The therapeutic methods and uses of the invention described herein, include, in another aspect, administering to a subject having a cancer (e.g., a hematologic cancer, e.g., a lymphoma (e.g., a non-Hodgkin's lymphoma (NHL), e.g., a relapsed or refractory DLBCL or a relapsed or refractory FL)) an effective amount of an anti-TIGIT antagonist antibody (e.g., an anti-TIGIT antagonist antibody described herein, e.g., tiragolumab) and an anti-CD20 antibody (e.g., rituximab) in a dosing regimen comprising at least a first and a second dosing cycle, wherein (a) the anti-TIGIT antagonist antibody is administered once every three weeks; and (b) the anti-CD20 antibody is administered once every week, thereby treating the subject.

The therapeutic methods and uses of the invention described herein, include, in another aspect, administering to a subject having a cancer (e.g., a hematologic cancer, e.g., a lymphoma (e.g., a non-Hodgkin's lymphoma (NHL), e.g., a relapsed or refractory DLBCL or a relapsed or refractory FL)) an effective amount of an anti-TIGIT antagonist antibody (e.g., an anti-TIGIT antagonist antibody described herein, e.g., tiragolumab) and an anti-CD20 antibody (e.g., rituximab) in a dosing regimen comprising at least a first, a second, and a third dosing cycle, wherein (a) the anti-TIGIT antagonist antibody is administered once every three weeks; and (b) the anti-CD20 antibody is administered once every week, thereby treating the subject.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In some aspects, the effective amount of the anti-CD20 antibody (e.g., rituximab) is a dose of between about 250 mg/m² to about 500 mg/m² (e.g., between about 250 mg/m² to about 450 mg/m², e.g., between about 250 mg/m² to about 400 mg/m², e.g., between about 300 mg/m² to about 400 mg/m², e.g., between about 325 mg/m² to about 400 mg/m², e.g., between about 350 mg/m² to about 400 mg/m², e.g., between about 350 mg/m² to about 375 mg/m², e.g., about 375±2 mg/m², about 375±1 mg/m², about 375±0.5 mg/m², about 375±0.2 mg/m², or about 375±0.1 mg/m², e.g., about 375 mg/m²). In some aspects, the effective amount of the anti-CD20 antibody (e.g., rituximab) is a dose of about 375 mg/m².

In any of the methods and uses of the invention, the anti-TIGIT antagonist antibody (e.g., an anti-TIGIT antagonist antibody as disclosed herein, e.g., tiragolumab) and the anti-CD20 antibody (e.g., rituximab) may be administered in a dosing regimen that includes at least a first and a second dosing cycle (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the anti-TIGIT antagonist antibody (e.g., an anti-TIGIT antagonist antibody as disclosed herein, e.g., tiragolumab) and the anti-CD20 antibody (e.g., rituximab) may be administered in a dosing regimen that includes at least a first, a second, and a third dosing cycle (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the anti-TIGIT antagonist antibody (e.g., an anti-TIGIT antagonist antibody as disclosed herein, e.g., tiragolumab) and the anti-CD20 antibody (e.g., rituximab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, each dosing cycle of the dosing regimen comprises a single dose of the anti-TIGIT antagonist antibody (e.g., an anti-TIGIT antagonist antibody as disclosed herein, e.g., tiragolumab). In some aspects, the anti-TIGIT antagonist antibody (e.g., an anti-TIGIT antagonist antibody as disclosed herein, e.g., tiragolumab) is administered on about day 1 (e.g., day 1±1 day) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab) is administered intravenously at a fixed dose of about 600 mg on day 2 of each 21 day cycle (i.e., at a fixed dose of about 600 mg every three weeks). Similarly in other aspects, the first dosing cycle comprises a first dose (C1D1), a second dose (C1D2), and a third dose (C1D3) of the anti-CD20 antibody; and the second dosing cycle comprises at least a first dose (C2D1) of the anti-CD20 antibody (e.g., rituximab). In some aspects, the dosing regimen comprises a total of four doses of the anti-CD20 antibody (e.g., rituximab). In some aspects, the method comprises administering the C1 D1 of the anti-CD20 antibody on or about day 1 (e.g., day 1±1 day) of the first dosing cycle, the C1 D2 of the anti-CD20 antibody on or about day 8 (e.g., day 8±1 day) of the first dosing cycle, and the C1 D3 of the anti-CD20 antibody on or about day 15 (e.g., day 15±1 day) of the first dosing cycle. For example, the C1 D1 of the anti-CD20 antibody is administered intravenously to the subject at a dose of 375 mg/m² on day 1 of the first dosing cycle, the C1 D2 of the anti-CD20 antibody is administered intravenously to the subject at a dose of 375 mg/m² on day 8 of the first dosing cycle, and the C1 D3 of the anti-CD20 antibody is administered intravenously to the subject at a dose of 375 mg/m² on day 15 of the first dosing cycle. In some aspects, the method comprises administering to the subject the C2D1 of the anti-CD20 antibody on or about day 1 (e.g., day 1±1 day) of the second dosing cycle. For example, the C2D1 of the anti-CD20 antibody (e.g., rituximab) is administered intravenously to the subject at a dose of 375 mg/m² on day 1 of the second dosing cycle. In some aspects, any of the C1 D1, C1 D2, C1 D3, and C2D1 of the anti-CD20 antibody (e.g., rituximab) may be split into two doses and administered to the subject over the course of two consecutive days.

In other aspects, the first dosing cycle comprises a first dose (C1D1), a second dose (C1D2), and a third dose (C1D3) of the anti-CD20 antibody; the second dosing cycle comprises a first dose (C2D1), a second dose (C2D2), and a third dose (C2D3) of the anti-CD20 antibody; and the third dosing cycle comprises at least a first dose (C3D1) and a second dose (C3D2) of the anti-CD20 antibody (e.g., rituximab). In some aspects, the dosing regimen comprises a total of eight doses of the anti-CD20 antibody (e.g., rituximab). In some aspects, the method comprises administering the C1 D1 of the anti-CD20 antibody on or about day 1 (e.g., day 1±1 day) of the first dosing cycle, the C1 D2 of the anti-CD20 antibody on or about day 8 (e.g., day 8±1 day) of the first dosing cycle, and the C1 D3 of the anti-CD20 antibody on or about day 15 (e.g., day 15±1 day) of the first dosing cycle. For example, the C1 D1 of the anti-CD20 antibody is administered intravenously to the subject at a dose of 375 mg/m² on day 1 of the first dosing cycle, the C1 D2 of the anti-CD20 antibody is administered intravenously to the subject at a dose of 375 mg/m² on day 8 of the first dosing cycle, and the C1 D3 of the anti-CD20 antibody is administered intravenously to the subject at a dose of 375 mg/m² on day 15 of the first dosing cycle. In some aspects, the method comprises administering to the subject the C2D1 of the anti-CD20 antibody on or about day 1 (e.g., day 1±1 day) of the second dosing cycle, the C2D2 of the anti-CD20 antibody on or about day 8 (e.g., day 8±1 day) of the second dosing cycle, and the C2D3 of the anti-CD20 antibody on or about day 15 (e.g., day 15±1 day) of the second dosing cycle. For example, the C2D1 of the anti-CD20 antibody (e.g., rituximab) is administered intravenously to the subject at a dose of 375 mg/m² on day 1 of the second dosing cycle, the C2D2 is administered intravenously to the subject at a dose of 375 mg/m² on day 8 of the second dosing cycle, and the C2D3 is administered intravenously to the subject at a dose of 375 mg/m² on day 15 of the second dosing cycle. In some aspects, the method comprises administering to the subject the C3D1 of the anti-CD20 antibody on or about day 1 (e.g., day 1±1 day) of the third dosing cycle and the C3D2 of the anti-CD20 antibody on or about day 8 (e.g., day 8±1 day) of the third dosing cycle. For example, the C3D1 of the anti-CD20 antibody (e.g., rituximab) is administered intravenously to the subject at a dose of 375 mg/m² on day 1 of the third dosing cycle and the C3D2 is administered intravenously to the subject at a dose of 375 mg/m² on day 8 of the third dosing cycle. In some aspects, any of the C1 D1, C1 D2, C1 D3, C2D1, C2D2, C2D3, C3D1, and C3D2 of the anti-CD20 antibody (e.g., rituximab) may be split into two doses and administered to the subject over the course of two consecutive days.

In some aspects, the anti-TIGIT antagonist antibody (e.g., an anti-TIGIT antagonist antibody as disclosed herein, e.g., tiragolumab) and the anti-CD20 antibody (e.g., rituximab) are both administered on the same day. For example, in some aspects, the anti-TIGIT antagonist antibody (e.g., an anti-TIGIT antagonist antibody as disclosed herein, e.g., tiragolumab) and the anti-CD20 antibody are both administered on or about day 1 (e.g., day 1±1 day) of each of dosing cycles 1 and 2. In other aspects, the anti-TIGIT antagonist antibody (e.g., an anti-TIGIT antagonist antibody as disclosed herein, e.g., tiragolumab) and the anti-CD20 antibody are both administered on or about day 1 (e.g., day 1±1 day) of each of dosing cycles 1, 2, and 3. In some aspects, the anti-TIGIT antagonist antibody (e.g., an anti-TIGIT antagonist antibody as disclosed herein, e.g., tiragolumab) is administered to the subject before the anti-CD20 antibody (e.g., rituximab). In some aspects, for example, following administration of the anti-TIGIT antagonist antibody and before administration of the anti-CD20 antibody, the method includes an intervening first observation period. In some aspects, the method further includes a second observation period following administration of the anti-CD20 antibody. In some aspects, the method includes both a first observation period following administration of the anti-TIGIT antagonist antibody and second observation period following administration of the anti-CD20 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-CD20 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-CD20 antibody during the first and second observation periods, respectively.

In some aspects, when the anti-TIGIT antagonist antibody (e.g., an anti-TIGIT antagonist antibody as disclosed herein, e.g., tiragolumab) and the anti-CD20 antibody (e.g., rituximab) are scheduled to be administered on the same day, the anti-CD20 antibody is administered on one day, and the anti-TIGIT antagonist antibody is administered on the next consecutive day. Accordingly, in some aspects, the anti-CD20 antibody (e.g. rituximab) is administered to the subject before the anti-TIGIT antagonist antibody (e.g., an anti-TIGIT antagonist antibody as disclosed herein, e.g., tiragolumab). For example, the anti-CD20 antibody may be administered on day 1, and the anti-TIGIT antagonist antibody may be administered on day 2. In some aspects, following administration of the anti-CD20 antibody and before administration of the anti-TIGIT antagonist antibody, the method includes an intervening first observation period. In some aspects, the method includes a second observation period following administration of the anti-TIGIT antagonist antibody. In some aspects, the method includes both a first observation period following administration of the anti-CD20 antibody and second observation period following administration of the anti-TIGIT antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD20 antibody and anti-TIGIT antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD20 antibody and anti-TIGIT antagonist antibody during the first and second observation periods, respectively.

In another aspect, the invention provides for a method of treating a subject having relapsed or refractory NHL by administering to the subject tiragolumab at a fixed dose of 600 mg and rituximab at a dose of 375 mg/m² in a dosing regimen comprising at least a first and a second dosing cycle, wherein the length of each dosing cycle is 21 days, and wherein (a) each dosing cycle comprises a single dose of tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; and (c) the second dosing cycle further comprises a single dose of rituximab administered on or about day 1 of the second dosing cycle, and wherein the dosing regimen comprises a total of four doses of rituximab.

In another aspect, the invention provides for a method of treating a subject having relapsed or refractory NHL by administering to the subject tiragolumab at a fixed dose of 600 mg and rituximab at a dose of 375 mg/m² in a dosing regimen comprising a first, a second, and a third dosing cycle, wherein the length of each dosing cycle is 21 days, and wherein: (a) each dosing cycle comprises a single dose of tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; (c) the second dosing cycle further comprises a first dose (C2D1), a second dose (C2D2), and a third dose (C2D3) of rituximab administered on or about days 1, 8, and 15 of the second dosing cycle; and (d) the third dosing cycle further comprises a first dose (C3D1) and a second dose (C3D2) of rituximab, wherein the C3D1 and the C3D2 are administered on or about days 1 and 8, respectively, of the third dosing cycle, and wherein the dosing regimen comprises a total of eight doses of rituximab.

In another aspect, the invention provides an anti-TIGIT antagonist antibody (e.g., an anti-TIGIT antagonist antibody disclosed herein, e.g., tiragolumab) and anti-CD20 antibody (e.g., rituximab) for use in a method of treating a subject having a cancer (e.g., a hematologic cancer, e.g., a lymphoma (e.g., a non-Hodgkin's lymphoma (NHL), e.g., a relapsed or refractory DLBCL or a relapsed or refractory FL)), wherein the method comprises administering to the subject an effective amount of an anti-TIGIT antagonist antibody (e.g., an anti-TIGIT antagonist antibody described herein, e.g., tiragolumab) and an anti-CD20 antibody (e.g., rituximab) in a dosing regimen comprising at least a first and a second dosing cycle, wherein (a) the anti-TIGIT antagonist antibody is administered once every three weeks; and (b) the anti-CD20 antibody is administered once every week. In some aspects, the dosing regimen comprises at least 12 dosing cycles. In some aspects, the dosing regimen comprises at least 16 dosing cycles.

In another aspect, the invention provides an anti-TIGIT antagonist antibody (e.g., an anti-TIGIT antagonist antibody disclosed herein, e.g., tiragolumab) and anti-CD20 antibody (e.g., rituximab) for use in a method of treating a subject having a cancer (e.g., a hematologic cancer, e.g., a lymphoma (e.g., a non-Hodgkin's lymphoma (NHL), e.g., a relapsed or refractory DLBCL or a relapsed or refractory FL)), wherein the method comprises administering to the subject an effective amount of an anti-TIGIT antagonist antibody (e.g., an anti-TIGIT antagonist antibody described herein, e.g., tiragolumab) and an anti-CD20 antibody (e.g., rituximab) in a dosing regimen comprising at least a first, a second, and a third dosing cycle, wherein: (a) the anti-TIGIT antagonist antibody is administered once every three weeks, and (b) the anti-CD20 antibody is administered once every week, thereby treating the subject. In some aspects, the dosing regimen comprises at least 16 dosing cycles.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In some aspects, the effective amount of the anti-CD20 antibody (e.g., rituximab) is a dose of between about 250 mg/m² to about 500 mg/m² (e.g., between about 250 mg/m² to about 450 mg/m², e.g., between about 250 mg/m² to about 400 mg/m², e.g., between about 300 mg/m² to about 400 mg/m², e.g., between about 325 mg/m² to about 400 mg/m², e.g., between about 350 mg/m² to about 400 mg/m², e.g., between about 350 mg/m² to about 375 mg/m², e.g., about 375±2 mg/m², about 375±1 mg/m², about 375±0.5 mg/m², about 375±0.2 mg/m², or about 375±0.1 mg/m², e.g., about 375 mg/m²). In some aspects, the effective amount of the anti-CD20 antibody (e.g., rituximab) is a dose of about 375 mg/m².

In any of the uses of the invention, the anti-TIGIT antagonist antibody (e.g., an anti-TIGIT antagonist antibody as disclosed herein, e.g., tiragolumab) and the anti-CD20 antibody (e.g., rituximab) are to be administered in a dosing regimen that includes at least a first and a second dosing cycle (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the anti-TIGIT antagonist antibody (e.g., an anti-TIGIT antagonist antibody as disclosed herein, e.g., tiragolumab) and the anti-CD20 antibody (e.g., rituximab) are to be administered in a dosing regimen that includes at least a first, a second, and a third dosing cycle (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles).

In other aspects, the dosing regimen includes at least 12 dosing cycles. In other aspects, the dosing regimen includes at least 16 dosing cycles. In some aspects, the dosing cycles of the anti-TIGIT antagonist antibody (e.g., an anti-TIGIT antagonist antibody as disclosed herein, e.g., tiragolumab) and the anti-CD20 antibody (e.g., rituximab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, each dosing cycle of the dosing regimen comprises a single dose of the anti-TIGIT antagonist antibody (e.g., an anti-TIGIT antagonist antibody as disclosed herein, e.g., tiragolumab). In some aspects, the anti-TIGIT antagonist antibody (e.g., an anti-TIGIT antagonist antibody as disclosed herein, e.g., tiragolumab) is to be administered on about day 1 (e.g., day 1±1 day) of each dosing cycle. For example, the anti-TIGIT antagonist antibody (e.g., an anti-TIGIT antagonist antibody as disclosed herein, e.g., tiragolumab) is to be administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab) is to be administered intravenously at a fixed dose of about 600 mg on day 2 of each 21 day cycle (i.e., at a fixed dose of about 600 mg every three weeks). Similarly in other aspects, the first dosing cycle comprises a first dose (C1D1), a second dose (C1D2), and a third dose (C1D3) of the anti-CD20 antibody (e.g., rituximab); and the second dosing cycle comprises at least a first dose (C2D1) of the anti-CD20 antibody (e.g., rituximab). In some aspects, the dosing regimen comprises a total of four doses of the anti-CD20 antibody (e.g., rituximab). In some aspects, the method comprises administering the C1 D1 of the anti-CD20 antibody on or about day 1 (e.g., day 1±1 day) of the first dosing cycle, the C1 D2 of the anti-CD20 antibody on or about day 8 (e.g., day 8±1 day) of the first dosing cycle, and the C1 D3 of the anti-CD20 antibody on or about day 15 (e.g., day 15±1 day) of the first dosing cycle. For example, the C1 D1 of the anti-CD20 antibody is to be administered intravenously to the subject at a dose of 375 mg/m² on day 1 of the first dosing cycle, the C1 D2 of the anti-CD20 antibody is to be administered intravenously to the subject at a dose of 375 mg/m² on day 8 of the first dosing cycle, and the C1 D3 of the anti-CD20 antibody is to be administered intravenously to the subject at a dose of 375 mg/m² on day 15 of the first dosing cycle. In some aspects, the method comprises administering to the subject the C2D1 of the anti-CD20 antibody on or about day 1 (e.g., day 1±1 day) of the second dosing cycle. For example, the C2D1 of the anti-CD20 antibody (e.g., rituximab) is to be administered intravenously to the subject at a dose of 375 mg/m² on day 1 of the second dosing cycle. In some aspects, any of the C1 D1, C1 D2, C1 D3, and C2D1 of the anti-CD20 antibody (e.g., rituximab) may be split into two doses and administered to the subject over the course of two consecutive days.

In other aspects, the first dosing cycle comprises a first dose (C1D1), a second dose (C1D2), and a third dose (C1D3) of the anti-CD20 antibody; the second dosing cycle comprises a first dose (C2D1), a second dose (C2D2), and a third dose (C2D3) of the anti-CD20 antibody; and the third dosing cycle comprises at least a first dose (C3D1) and a second dose (C3D2) of the anti-CD20 antibody (e.g., rituximab). In some aspects, the dosing regimen comprises a total of eight doses of the anti-CD20 antibody (e.g., rituximab). In some aspects, the method comprises administering the C1 D1 of the anti-CD20 antibody on or about day 1 (e.g., day 1±1 day) of the first dosing cycle, the C1 D2 of the anti-CD20 antibody on or about day 8 (e.g., day 8±1 day) of the first dosing cycle, and the C1 D3 of the anti-CD20 antibody on or about day 15 (e.g., day 15±1 day) of the first dosing cycle. For example, the C1 D1 of the anti-CD20 antibody is to be administered intravenously to the subject at a dose of 375 mg/m² on day 1 of the first dosing cycle, the C1 D2 of the anti-CD20 antibody is to be administered intravenously to the subject at a dose of 375 mg/m² on day 8 of the first dosing cycle, and the C1 D3 of the anti-CD20 antibody is to be administered intravenously to the subject at a dose of 375 mg/m² on day 15 of the first dosing cycle. In some aspects, the method comprises administering to the subject the C2D1 of the anti-CD20 antibody on or about day 1 (e.g., day 1±1 day) of the second dosing cycle, the C2D2 of the anti-CD20 antibody on or about day 8 (e.g., day 8±1 day) of the second dosing cycle, and the C2D3 of the anti-CD20 antibody on or about day 15 (e.g., day 15±1 day) of the second dosing cycle. For example, the C2D1 of the anti-CD20 antibody (e.g., rituximab) is to be administered intravenously to the subject at a dose of 375 mg/m² on day 1 of the second dosing cycle, the C2D2 is to be administered intravenously to the subject at a dose of 375 mg/m² on day 8 of the second dosing cycle, and the C2D3 is to be administered intravenously to the subject at a dose of 375 mg/m² on day 15 of the second dosing cycle. In some aspects, the method comprises administering to the subject the C3D1 of the anti-CD20 antibody on or about day 1 (e.g., day 1±1 day) of the third dosing cycle and the C3D2 of the anti-CD20 antibody on or about day 8 (e.g., day 8±1 day) of the third dosing cycle. For example, the C3D1 of the anti-CD20 antibody (e.g., rituximab) is to be administered intravenously to the subject at a dose of 375 mg/m² on day 1 of the third dosing cycle and the C3D2 is to be administered intravenously to the subject at a dose of 375 mg/m² on day 8 of the third dosing cycle. In some aspects, any of the C1 D1, C1 D2, C1 D3, C2D1, C2D2, C2D3, C3D1, and C3D2 of the anti-CD20 antibody (e.g., rituximab) may be split into two doses and administered to the subject over the course of two consecutive days.

In some aspects, the anti-TIGIT antagonist antibody (e.g., an anti-TIGIT antagonist antibody as disclosed herein, e.g., tiragolumab) and the anti-CD20 antibody (e.g., rituximab) are both to be administered on the same day. For example, in some aspects, the anti-TIGIT antagonist antibody (e.g., an anti-TIGIT antagonist antibody as disclosed herein, e.g., tiragolumab) and the anti-CD20 antibody are both to be administered on or about day 1 (e.g., day 1±1 day) of each of dosing cycles 1 and 2. In other aspects, the anti-TIGIT antagonist antibody (e.g., an anti-TIGIT antagonist antibody as disclosed herein, e.g., tiragolumab) and the anti-CD20 antibody are both to be administered on or about day 1 (e.g., day 1±1 day) of each of dosing cycles 1, 2, and 3. In some aspects, the anti-TIGIT antagonist antibody (e.g., an anti-TIGIT antagonist antibody as disclosed herein, e.g., tiragolumab) is to be administered to the subject before the anti-CD20 antibody (e.g., rituximab). In some aspects, for example, following administration of the anti-TIGIT antagonist antibody and before administration of the anti-CD20 antibody, the method includes an intervening first observation period. In some aspects, the method further includes a second observation period following administration of the anti-CD20 antibody. In some aspects, the method includes both a first observation period following administration of the anti-TIGIT antagonist antibody and second observation period following administration of the anti-CD20 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-CD20 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-CD20 antibody during the first and second observation periods, respectively.

In some aspects, when the anti-TIGIT antagonist antibody (e.g., an anti-TIGIT antagonist antibody as disclosed herein, e.g., tiragolumab) and the anti-CD20 antibody (e.g., rituximab) are scheduled to be administered on the same day, the anti-CD20 antibody is to be administered on one day, and the anti-TIGIT antagonist antibody is to be administered on the next consecutive day. Accordingly, in some aspects, the anti-CD20 antibody (e.g. rituximab) is to be administered to the subject before the anti-TIGIT antagonist antibody (e.g., an anti-TIGIT antagonist antibody as disclosed herein, e.g., tiragolumab). For example, the anti-CD20 antibody is to be administered on day 1, and the anti-TIGIT antagonist antibody is to be administered on day 2. In some aspects, following administration of the anti-CD20 antibody and before administration of the anti-TIGIT antagonist antibody, the method includes an intervening first observation period. In some aspects, the method includes a second observation period following administration of the anti-TIGIT antagonist antibody. In some aspects, the method includes both a first observation period following administration of the anti-CD20 antibody and second observation period following administration of the anti-TIGIT antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD20 antibody and anti-TIGIT antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD20 antibody and anti-TIGIT antagonist antibody during the first and second observation periods, respectively.

In another aspect, the invention provides for tiragolumab and rituximab for use in a method of treating a subject having relapsed or refractory NHL, wherein the method comprises administering to the subject tiragolumab at a fixed dose of 600 mg and rituximab at a dose of 375 mg/m² in a dosing regimen comprising at least a first and a second dosing cycle, wherein the length of each dosing cycle is 21 days, and wherein (a) each dosing cycle comprises a single dose of tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; and (c) the second dosing cycle further comprises a single dose of rituximab administered on or about day 1 of the second dosing cycle, and wherein the dosing regimen comprises a total of four doses of rituximab. In some aspects, the dosing regimen comprises at least 12 dosing cycles. In some aspects, the dosing regimen comprises at least 16 dosing cycles.

In another aspect, the invention provides for tiragolumab and rituximab for use in a method of treating a subject having relapsed or refractory NHL, wherein the method comprises administering to the subject tiragolumab at a fixed dose of 600 mg and rituximab at a dose of 375 mg/m² in a dosing regimen comprising at least a first, a second, and a third dosing cycle, wherein the length of each dosing cycle is 21 days, and wherein (a) each dosing cycle comprises a single dose of tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; (c) the second dosing cycle further comprises a first dose (C2D1), a second dose (C2D2), and a third dose (C2D3) of rituximab administered on or about days 1, 8, and 15 of the second dosing cycle; and (d) the third dosing cycle further comprises a first dose (C3D1) and a second dose (C3D2) of rituximab, wherein the C3D1 and the C3D2 are administered on or about days 1 and 8, respectively, of the third dosing cycle, and wherein the dosing regimen comprises a total of eight doses of rituximab. In some aspects, the dosing regimen comprises at least 12 dosing cycles. In some aspects, the dosing regimen comprises at least 16 dosing cycles.

In another aspect, the invention provides an anti-TIGIT antagonist antibody (e.g., an anti-TIGIT antagonist antibody disclosed herein, e.g., tiragolumab) in the manufacture or preparation of a medicament for use in a method of treating a subject having a cancer (e.g., a hematologic cancer, e.g., a myeloma (e.g., a multiple myeloma (MM), e.g., a relapsed or refractory MM) or a lymphoma (e.g., a non-Hodgkin's lymphoma (NHL), e.g., a relapsed or refractory DLBCL or a relapsed or refractory FL)), wherein the method comprises administering to the subject an effective amount of the medicament comprising the anti-TIGIT antagonist antibody in combination with an anti-CD20 antibody (e.g., rituximab) in a dosing regimen comprising at least a first and a second dosing cycle, wherein (a) the medicament comprising the anti-TIGIT antagonist antibody is administered once every three weeks; and (b) the anti-CD20 antibody is administered once every week. In some aspects, the dosing regimen comprises at least 12 dosing cycles. In some aspects, the dosing regimen comprises at least 16 dosing cycles.

In another aspect, the invention provides an anti-TIGIT antagonist antibody (e.g., an anti-TIGIT antagonist antibody disclosed herein, e.g., tiragolumab) in the manufacture or preparation of a medicament for use in a method of treating a subject having a cancer (e.g., a hematologic cancer, e.g., a myeloma (e.g., a multiple myeloma (MM), e.g., a relapsed or refractory MM) or a lymphoma (e.g., a non-Hodgkin's lymphoma (NHL), e.g., a relapsed or refractory DLBCL or a relapsed or refractory FL)), wherein the method comprises administering to the subject an effective amount of the medicament comprising the anti-TIGIT antagonist antibody in combination with an anti-CD20 antibody (e.g., rituximab) in a dosing regimen comprising at least a first, a second, and a third dosing cycle, wherein (a) the medicament comprising the anti-TIGIT antagonist antibody is administered once every three weeks; and (b) the anti-CD20 antibody is administered once every week. In some aspects, the dosing regimen comprises at least 12 dosing cycles. In some aspects, the dosing regimen comprises at least 16 dosing cycles.

In another aspect, the invention provides an anti-CD20 antibody (e.g., rituximab) in the manufacture or preparation of a medicament for use in a method of treating a subject having a cancer (e.g., a hematologic cancer, e.g., a lymphoma (e.g., a non-Hodgkin's lymphoma (NHL), e.g., a relapsed or refractory DLBCL or a relapsed or refractory FL)), wherein the method comprises administering to the subject an effective amount of an anti-TIGIT antagonist antibody in combination with the medicament comprising anti-CD20 antibody (e.g., rituximab) in a dosing regimen comprising at least a first and a second dosing cycle, wherein (a) the anti-TIGIT antagonist antibody is administered once every three weeks; and (b) the medicament comprising the anti-CD20 antibody is administered once every week. In some aspects, the dosing regimen comprises at least 12 dosing cycles. In some aspects, the dosing regimen comprises at least 16 dosing cycles.

In another aspect, the invention provides an anti-CD20 antibody (e.g., rituximab) in the manufacture or preparation of a medicament for use in a method of treating a subject having a cancer (e.g., a hematologic cancer, e.g., a lymphoma (e.g., a non-Hodgkin's lymphoma (NHL), e.g., a relapsed or refractory DLBCL or a relapsed or refractory FL)), wherein the method comprises administering to the subject an effective amount of an anti-TIGIT antagonist antibody in combination with the medicament comprising anti-CD20 antibody (e.g., rituximab) in a dosing regimen comprising at least a first, a second, and a third dosing cycle, wherein (a) the anti-TIGIT antagonist antibody is administered once every three weeks; and (b) the medicament comprising the anti-CD20 antibody is administered once every week. In some aspects, the dosing regimen comprises at least 12 dosing cycles. In some aspects, the dosing regimen comprises at least 16 dosing cycles.

In another aspect, the invention provides an anti-TIGIT antagonist antibody (e.g., an anti-TIGIT antagonist antibody disclosed herein, e.g., tiragolumab) and an anti-CD20 antagonist antibody (e.g., rituximab) in the manufacture or preparation of medicaments for use in a method of treating a subject having a cancer (e.g., a hematologic cancer, e.g., a lymphoma (e.g., a non-Hodgkin's lymphoma (NHL), e.g., a relapsed or refractory DLBCL or a relapsed or refractory FL)), wherein the method comprises administering to the subject an effective amount of the medicament comprising the anti-TIGIT antagonist antibody and the medicament comprising the anti-CD20 antibody (e.g., rituximab) in a dosing regimen comprising at least a first and a second dosing cycle, wherein (a) the medicament comprising the anti-TIGIT antagonist antibody is administered once every three weeks; and (b) the medicament comprising the anti-CD20 antibody is administered once every week. In some aspects, the dosing regimen comprises at least 12 dosing cycles. In some aspects, the dosing regimen comprises at least 16 dosing cycles.

In another aspect, the invention provides an anti-TIGIT antagonist antibody (e.g., an anti-TIGIT antagonist antibody disclosed herein, e.g., tiragolumab) and an anti-CD20 antagonist antibody (e.g., rituximab) in the manufacture or preparation of medicaments for use in a method of treating a subject having a cancer (e.g., a hematologic cancer, e.g., a lymphoma (e.g., a non-Hodgkin's lymphoma (NHL), e.g., a relapsed or refractory DLBCL or a relapsed or refractory FL)), wherein the method comprises administering to the subject an effective amount of the medicament comprising the anti-TIGIT antagonist antibody and the medicament comprising the anti-CD20 antibody (e.g., rituximab) in a dosing regimen comprising at least a first, a second, and a third dosing cycle, wherein (a) the medicament comprising the anti-TIGIT antagonist antibody is administered once every three weeks; and (b) the medicament comprising the anti-CD20 antibody is administered once every week. In some aspects, the dosing regimen comprises at least 12 dosing cycles. In some aspects, the dosing regimen comprises at least 16 dosing cycles.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In some aspects, the effective amount of the anti-CD20 antibody (e.g., rituximab) is a dose of between about 250 mg/m² to about 500 mg/m² (e.g., between about 250 mg/m² to about 450 mg/m², e.g., between about 250 mg/m² to about 400 mg/m², e.g., between about 300 mg/m² to about 400 mg/m², e.g., between about 325 mg/m² to about 400 mg/m², e.g., between about 350 mg/m² to about 400 mg/m², e.g., between about 350 mg/m² to about 375 mg/m², e.g., about 375±2 mg/m², about 375±1 mg/m², about 375±0.5 mg/m², about 375±0.2 mg/m², or about 375±0.1 mg/m², e.g., about 375 mg/m²). In some aspects, the effective amount of the anti-CD20 antibody (e.g., rituximab) is a dose of about 375 mg/m².

In any of the uses of the invention, (a) the anti-CD20 antibody (e.g., rituximab) and the medicament comprising the anti-TIGIT antagonist antibody (e.g., an anti-TIGIT antagonist antibody as disclosed herein, e.g., tiragolumab), (b) the anti-TIGIT antagonist antibody (e.g., an anti-TIGIT antagonist antibody as disclosed herein, e.g., tiragolumab) and the medicament comprising the anti-CD20 antibody (e.g., rituximab), or (c) the medicament comprising the anti-TIGIT antagonist antibody (e.g., an anti-TIGIT antagonist antibody as disclosed herein, e.g., tiragolumab) and the medicament comprising the anti-CD20 antibody (e.g., rituximab) are to be administered in a dosing regimen that includes at least a first and a second dosing cycle (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, (a) the anti-CD20 antibody (e.g., rituximab) and the medicament comprising the anti-TIGIT antagonist antibody (e.g., an anti-TIGIT antagonist antibody as disclosed herein, e.g., tiragolumab), (b) the anti-TIGIT antagonist antibody (e.g., an anti-TIGIT antagonist antibody as disclosed herein, e.g., tiragolumab) and the medicament comprising the anti-CD20 antibody (e.g., rituximab), or (c) the medicament comprising the anti-TIGIT antagonist antibody (e.g., an anti-TIGIT antagonist antibody as disclosed herein, e.g., tiragolumab) and the medicament comprising the anti-CD20 antibody (e.g., rituximab) are to be administered in a dosing regimen that includes at least a first, a second, and a third dosing cycle (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 12 dosing cycles. In other aspects, the dosing regimen includes at least 16 dosing cycles. In some aspects, the dosing cycles of the medicament comprising (a) the anti-CD20 antibody (e.g., rituximab) and the medicament comprising the anti-TIGIT antagonist antibody (e.g., an anti-TIGIT antagonist antibody as disclosed herein, e.g., tiragolumab), (b) the anti-TIGIT antagonist antibody (e.g., an anti-TIGIT antagonist antibody as disclosed herein, e.g., tiragolumab) and the medicament comprising the anti-CD20 antibody (e.g., rituximab), or (c) the medicament comprising the anti-TIGIT antagonist antibody (e.g., an anti-TIGIT antagonist antibody as disclosed herein, e.g., tiragolumab) and the medicament comprising the anti-CD20 antibody (e.g., rituximab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, each dosing cycle of the dosing regimen comprises a single dose of the anti-TIGIT antagonist antibody (e.g., an anti-TIGIT antagonist antibody as disclosed herein, e.g., tiragolumab), or medicament thereof. In some aspects, the anti-TIGIT antagonist antibody (e.g., an anti-TIGIT antagonist antibody as disclosed herein, e.g., tiragolumab), or medicament thereof, is to be administered on about day 1 (e.g., day 1±1 day) of each dosing cycle. For example, the anti-TIGIT antagonist antibody (e.g., an anti-TIGIT antagonist antibody as disclosed herein, e.g., tiragolumab), or medicament thereof, is to be administered intravenously at a fixed dose of about 600 mg on day 1 of each 21 day cycle (i.e., at a fixed dose of about 600 mg every three weeks). In another aspect, of the anti-TIGIT antagonist antibody (e.g., an anti-TIGIT antagonist antibody as disclosed herein, e.g., tiragolumab), or medicament thereof, is to be administered intravenously at a fixed dose of about 600 mg on day 2 of each 21 day cycle (i.e., at a fixed dose of about 600 mg every three weeks). Similarly in other aspects, the first dosing cycle comprises a first dose (C1D1), a second dose (C1D2), and a third dose (C1D3) of the anti-CD20 antibody (e.g., rituximab), or medicament thereof; and the second dosing cycle comprises at least a first dose (C2D1) of the anti-CD20 antibody (e.g., rituximab), or medicament thereof. In some aspects, the dosing regimen comprises a total of four doses of the anti-CD20 antibody (e.g., rituximab), or medicament thereof. In some aspects, the method comprises administering the C1 D1 of the anti-CD20 antibody (e.g., rituximab), or medicament thereof, on or about day 1 (e.g., day 1±1 day) of the first dosing cycle, the C1 D2 of the anti-CD20 antibody (e.g., rituximab), or medicament thereof, on or about day 8 (e.g., day 8±1 day) of the first dosing cycle, and the C1 D3 of the anti-CD20 antibody (e.g., rituximab), or medicament thereof on or about day 15 (e.g., day 15±1 day) of the first dosing cycle. For example, the C1 D1 of the anti-CD20 antibody (e.g., rituximab), or medicament thereof, is to be administered intravenously to the subject at a dose of 375 mg/m² on day 1 of the first dosing cycle, the C1 D2 of the anti-CD20 antibody (e.g., rituximab), or medicament thereof, is to be administered intravenously to the subject at a dose of 375 mg/m² on day 8 of the first dosing cycle, and the C1 D3 of the anti-CD20 antibody (e.g., rituximab), or medicament thereof, is to be administered intravenously to the subject at a dose of 375 mg/m² on day 15 of the first dosing cycle. In some aspects, the method comprises administering to the subject the C2D1 of the anti-CD20 antibody (e.g., rituximab), or medicament thereof, on or about day 1 (e.g., day 1±1 day) of the second dosing cycle. For example, the C2D1 of the anti-CD20 antibody (e.g., rituximab), or medicament thereof, is to be administered intravenously to the subject at a dose of 375 mg/m² on day 1 of the second dosing cycle. In some aspects, any of the C1 D1, C1 D2, C1 D3, and C2D1 of the anti-CD20 antibody (e.g., rituximab), or medicament thereof, may be split into two doses and administered to the subject over the course of two consecutive days.

In other aspects, the first dosing cycle comprises a first dose (C1D1), a second dose (C1D2), and a third dose (C1D3) of the anti-CD20 antibody (e.g., rituximab), or medicament thereof; the second dosing cycle comprises a first dose (C2D1), a second dose (C2D2), and a third dose (C2D3) of the anti-CD20 antibody (e.g., rituximab), or medicament thereof; and the third dosing cycle comprises at least a first dose (C3D1) and a second dose (C3D2) of the anti-CD20 antibody (e.g., rituximab), or medicament thereof. In some aspects, the dosing regimen comprises a total of eight doses of the anti-CD20 antibody (e.g., rituximab), or medicament thereof. In some aspects, the method comprises administering the C1 D1 of the anti-CD20 antibody (e.g., rituximab), or medicament thereof, on or about day 1 (e.g., day 1±1 day) of the first dosing cycle, the C1 D2 of the anti-CD20 antibody (e.g., rituximab), or medicament thereof, on or about day 8 (e.g., day 8±1 day) of the first dosing cycle, and the C1 D3 of the anti-CD20 antibody (e.g., rituximab), or medicament thereof on or about day 15 (e.g., day 15±1 day) of the first dosing cycle. For example, the C1 D1 of the anti-CD20 antibody (e.g., rituximab), or medicament thereof, is to be administered intravenously to the subject at a dose of 375 mg/m² on day 1 of the first dosing cycle, the C1 D2 of the anti-CD20 antibody (e.g., rituximab), or medicament thereof, is to be administered intravenously to the subject at a dose of 375 mg/m² on day 8 of the first dosing cycle, and the C1 D3 of the anti-CD20 antibody (e.g., rituximab), or medicament thereof, is to be administered intravenously to the subject at a dose of 375 mg/m² on day 15 of the first dosing cycle. In some aspects, the method comprises administering to the subject the C2D1 of the anti-CD20 antibody (e.g., rituximab), or medicament thereof, on or about day 1 (e.g., day 1±1 day) of the second dosing cycle, the C2D2 of the anti-CD20 antibody (e.g., rituximab), or medicament thereof, on or about day 8 (e.g., day 8±1 day) of the second dosing cycle, and the C2D3 of the anti-CD20 antibody (e.g., rituximab), or medicament thereof, on or about day 15 (e.g., day 15±1 day) of the second dosing cycle. For example, the C2D1 of the anti-CD20 antibody (e.g., rituximab), or medicament thereof, is to be administered intravenously to the subject at a dose of 375 mg/m² on day 1 of the second dosing cycle, the C2D2 of the anti-CD20 antibody (e.g., rituximab), or medicament thereof, is to be administered intravenously to the subject at a dose of 375 mg/m² on day 8 of the second dosing cycle, and the C2D3 of the anti-CD20 antibody (e.g., rituximab), or medicament thereof, is to be administered intravenously to the subject at a dose of 375 mg/m² on day 15 of the second dosing cycle. In some aspects, the method comprises administering to the subject the C3D1 of the anti-CD20 antibody (e.g., rituximab), or medicament thereof, on or about day 1 (e.g., day 1±1 day) of the third dosing cycle and the C3D2 of the anti-CD20 antibody (e.g., rituximab), or medicament thereof, on or about day 8 (e.g., day 8±1 day) of the third dosing cycle. For example, the C3D1 of the anti-CD20 antibody (e.g., rituximab), or medicament thereof, is to be administered intravenously to the subject at a dose of 375 mg/m² on day 1 of the third dosing cycle and the C3D2 of the anti-CD20 antibody (e.g., rituximab), or medicament thereof, is to be administered intravenously to the subject at a dose of 375 mg/m² on day 8 of the third dosing cycle. In some aspects, any of the C1 D1, C1 D2, C1 D3, C2D1, C2D2, C2D3, C3D1, and C3D2 of the anti-CD20 antibody (e.g., rituximab), or medicament thereof, may be split into two doses and administered to the subject over the course of two consecutive days.

In some aspects, the anti-TIGIT antagonist antibody (e.g., an anti-TIGIT antagonist antibody as disclosed herein, e.g., tiragolumab) and the anti-CD20 antibody (e.g., rituximab) are both to be administered on the same day. For example, in some aspects, the anti-TIGIT antagonist antibody (e.g., an anti-TIGIT antagonist antibody as disclosed herein, e.g., tiragolumab) and the anti-CD20 antibody are both to be administered on or about day 1 (e.g., day 1±1 day) of each of dosing cycles 1 and 2. In other aspects, the anti-TIGIT antagonist antibody (e.g., an anti-TIGIT antagonist antibody as disclosed herein, e.g., tiragolumab) and the anti-CD20 antibody are both to be administered on or about day 1 (e.g., day 1±1 day) of each of dosing cycles 1, 2, and 3. In some aspects, the anti-TIGIT antagonist antibody (e.g., an anti-TIGIT antagonist antibody as disclosed herein, e.g., tiragolumab) is to be administered to the subject before the anti-CD20 antibody (e.g., rituximab). In some aspects, for example, following administration of the anti-TIGIT antagonist antibody and before administration of the anti-CD20 antibody, the method includes an intervening first observation period. In some aspects, the method further includes a second observation period following administration of the anti-CD20 antibody. In some aspects, the method includes both a first observation period following administration of the anti-TIGIT antagonist antibody and second observation period following administration of the anti-CD20 antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and the anti-CD20 antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and the anti-CD20 antibody during the first and second observation periods, respectively.

In some aspects, when the anti-TIGIT antagonist antibody (e.g., an anti-TIGIT antagonist antibody as disclosed herein, e.g., tiragolumab) and the anti-CD20 antibody (e.g., rituximab) are scheduled to be administered on the same day, the anti-CD20 antibody is administered on one day, and the anti-TIGIT antagonist antibody is administered on the next consecutive day. Accordingly, in some aspects, the anti-CD20 antibody (e.g. rituximab) is administered to the subject before the anti-TIGIT antagonist antibody (e.g., an anti-TIGIT antagonist antibody as disclosed herein, e.g., tiragolumab). For example, the anti-CD20 antibody may be administered on day 1, and the anti-TIGIT antagonist antibody may be administered on day 2. In some aspects, following administration of the anti-CD20 antibody and before administration of the anti-TIGIT antagonist antibody, the method includes an intervening first observation period. In some aspects, the method includes a second observation period following administration of the anti-TIGIT antagonist antibody. In some aspects, the method includes both a first observation period following administration of the anti-CD20 antibody and second observation period following administration of the anti-TIGIT antagonist antibody. In some aspects, the first and second observation periods are each between about 30 minutes to about 60 minutes in length. In aspects in which the first and second observation periods are each about 60 minutes in length, the method may include recording the subject's vital signs (e.g., pulse rate, respiratory rate, blood pressure, and temperature) at about 30±10 minutes after administration of the anti-CD20 antibody and anti-TIGIT antagonist antibody during the first and second observation periods, respectively. In aspects in which the first and second observation periods are each about 30 minutes in length, the method may include recording the subject's vital signs (e.g., pulse rate, respiratory rate, blood pressure, and temperature) at about 15±10 minutes after administration of the anti-CD20 antibody and anti-TIGIT antagonist antibody during the first and second observation periods, respectively.

In another aspect, the invention provides for tiragolumab in the manufacture or preparation of a medicament for use in a method of treating a subject having relapsed or refractory NHL, wherein the method comprises administering to the subject the medicament comprising tiragolumab at a fixed dose of 600 mg in combination with rituximab at a dose of 375 mg/m² in a dosing regimen comprising at least a first and a second dosing cycle, wherein the length of each dosing cycle is 21 days, and wherein (a) each dosing cycle comprises a single dose of the medicament comprising tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; and (c) the second dosing cycle further comprises a single dose of rituximab administered on or about day 1 of the second dosing cycle, and wherein the dosing regimen comprises a total of four doses of rituximab. In some aspects, the dosing regimen comprises at least 12 dosing cycles. In some aspects, the dosing regimen comprises at least 16 dosing cycles.

In another aspect, the invention provides for tiragolumab in the manufacture or preparation of a medicament for use in a method of treating a subject having relapsed or refractory NHL, wherein the method comprises administering to the subject the medicament comprising tiragolumab at a fixed dose of 600 mg in combination with rituximab at a dose of 375 mg/m² in a dosing regimen comprising at least a first, a second, and a third dosing cycle, wherein the length of each dosing cycle is 21 days, and wherein (a) each dosing cycle comprises a single dose of the medicament comprising tiragolumab administered on or about day 1 of each dosing cycle; (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle; (c) the second dosing cycle further comprises a first dose (C2D1), a second dose (C2D2), and a third dose (C2D3) of rituximab, wherein the C2D1, C2D2, and C2D3 administered on or about days 1, 8, and 15, respectively, of the second dosing cycle; and (d) the third dosing cycle further comprises a first dose (C3D1) and a second dose (C3D2) of rituximab, wherein the C3D1 and C3D2 administered on or about days 1 and 8, respectively, of the third dosing cycle; and wherein the dosing regimen comprises a total of eight doses of rituximab. In some aspects, the dosing regimen comprises at least 12 dosing cycles. In some aspects, the dosing regimen comprises at least 16 dosing cycles.

In another aspect, the invention provides for rituximab in the manufacture or preparation of a medicament for use in a method of treating a subject having relapsed or refractory NHL, wherein the method comprises administering to the subject tiragolumab at a fixed dose of 600 mg in combination with the medicament comprising rituximab at a dose of 375 mg/m² in a dosing regimen comprising at least a first and a second dosing cycle, wherein the length of each dosing cycle is 21 days, and wherein (a) each dosing cycle comprises a single dose of tiragolumab administered on or about day 1 of each dosing cycle; (b) the first dosing cycle comprises a first dose (C1D1), a second dose (C1D2), and a third dose (C1D3) of the medicament comprising rituximab, wherein the C1 D1, the C1 D2, and the C1 D3 are administered on or about days 1, 8, and 15, respectively, of the first dosing cycle; and (c) the second dosing cycle further comprises a single dose of the medicament comprising rituximab administered on or about day 1 of the second dosing cycle, and wherein the dosing regimen comprises a total of four doses of the medicament comprising rituximab. In some aspects, the dosing regimen comprises at least 12 dosing cycles. In some aspects, the dosing regimen comprises at least 16 dosing cycles.

In another aspect, the invention provides for rituximab in the manufacture or preparation of a medicament for use in a method of treating a subject having relapsed or refractory NHL, wherein the method comprises administering to the subject tiragolumab at a fixed dose of 600 mg in combination with the medicament comprising rituximab at a dose of 375 mg/m² in a dosing regimen comprising at least a first, a second, and a third dosing cycle, wherein the length of each dosing cycle is 21 days, and wherein (a) each dosing cycle comprises a single dose of tiragolumab administered on or about day 1 of each dosing cycle; (b) the first dosing cycle comprises a first dose (C1D1), a second dose (C1D2), and a third dose (C1D3) of the medicament comprising rituximab, wherein the C1 D1, the C1 D2, and the C1 D3 are administered on or about days 1, 8, and 15, respectively, of the first dosing cycle; (c) the second dosing cycle further comprises a first dose (C2D1), a second dose (C2D2), and a third dose (C2D3) of the medicament comprising rituximab, wherein the C2D1, C2D2, and C2D3 are administered on or about days 1, 8, and 15, respectively, of the second dosing cycle; and (d) the third dosing cycle further comprises a first dose (C3D1) and a second dose (C3D2) of the medicament comprising rituximab, wherein the C3D1 and C3D2 are administered on or about days 1 and 8 respectively, of the third dosing cycle; and wherein the dosing regimen comprises a total of eight doses of the medicament comprising rituximab. In some aspects, the dosing regimen comprises at least 12 dosing cycles. In some aspects, the dosing regimen comprises at least 16 dosing cycles.

In another aspect, the invention provides for tiragolumab and rituximab in the manufacture or preparation of a medicament for use in a method of treating a subject having relapsed or refractory NHL, wherein the method comprises administering to the subject the medicament comprising tiragolumab at a fixed dose of 600 mg in combination with the medicament comprising rituximab at a dose of 375 mg/m² in a dosing regimen comprising at least a first and a second dosing cycle, wherein the length of each dosing cycle is 21 days, and wherein (a) each dosing cycle comprises a single dose of the medicament comprising tiragolumab administered on or about day 1 of each dosing cycle; (b) the first dosing cycle comprises a first dose (C1D1), a second dose (C1D2), and a third dose (C1D3) of the medicament comprising rituximab, wherein the C1 D1, the C1 D2, and the C1 D3 are administered on or about days 1, 8, and 15, respectively, of the first dosing cycle; and (c) the second dosing cycle further comprises a single dose of the medicament comprising rituximab administered on or about day 1 of the second dosing cycle, and wherein the dosing regimen comprises a total of four doses of the medicament comprising rituximab. In some aspects, the dosing regimen comprises at least 12 dosing cycles. In some aspects, the dosing regimen comprises at least 16 dosing cycles.

In another aspect, the invention provides for tiragolumab and rituximab in the manufacture or preparation of a medicament for use in a method of treating a subject having relapsed or refractory NHL, wherein the method comprises administering to the subject the medicament comprising tiragolumab at a fixed dose of 600 mg in combination with the medicament comprising rituximab at a dose of 375 mg/m² in a dosing regimen comprising at least a first, a second, and a third dosing cycle, wherein the length of each dosing cycle is 21 days, and wherein (a) each dosing cycle comprises a single dose of the medicament comprising tiragolumab administered on or about day 1 of each dosing cycle; (b) the first dosing cycle comprises a first dose (C1D1), a second dose (C1D2), and a third dose (C1D3) of the medicament comprising rituximab, wherein the C1 D1, the C1 D2, and the C1 D3 are administered on or about days 1, 8, and 15, respectively, of the first dosing cycle; (c) the second dosing cycle further comprises a first dose (C2D1), a second dose (C2D2), and a third dose (C2D3) of the medicament comprising rituximab, wherein the C2D1, C2D2, and C2D3 are administered on or about days 1, 8, and 15, respectively, of the second dosing cycle; and (d) the third dosing cycle further comprises at least a first dose (C3D1) and a second dose (C3D2) of the medicament comprising rituximab, wherein the C3D1 and C3D2 are administered on or about days 1 and 8, respectively, of the third dosing cycle; and wherein the dosing regimen comprises a total of eight doses of the medicament comprising rituximab. In some aspects, the dosing regimen comprises at least 12 dosing cycles. In some aspects, the dosing regimen comprises at least 16 dosing cycles.

In any of the methods, uses, and compositions for use described herein, the subject has an infusion-related reaction to the anti-TIGIT antagonist antibody (e.g., an anti-TIGIT antagonist antibody as disclosed herein, e.g., tiragolumab). In such aspects, the method further includes administering to the subject an antihistamine (e.g., diphenhydramine) and/or an antipyretic (e.g., acetaminophen) prior to a subsequent administration of the anti-TIGIT antagonist antibody (e.g., an anti-TIGIT antagonist antibody as disclosed herein, e.g., tiragolumab).

In any of the methods, uses, and compositions for use described herein, the anti-TIGIT antagonist antibody (e.g., an anti-TIGIT antagonist antibody as disclosed herein, e.g., tiragolumab), or medicament thereof, and anti-CD38 antibody (e.g., daratumumab), or medicament thereof, is for treating a subject having a hematologic cancer. In some aspects, the hematologic cancer is a myeloma. In some aspects the myeloma is a multiple myeloma (MM). In some aspects, the MM is a relapsed or refractory MM. In some aspects, the subject has a recurrent or relapsed MM for which no established therapy for MM is appropriate and available or be intolerant to those established therapies. In some aspects, the subject has received at least three prior therapeutic regimens (e.g., including at least one prior regimen containing a proteasome inhibitor, at least one containing an immunomodulatory drug, and at least one containing an anti-CD38 antibody). In some aspects, measurable disease is defined as a subject having one or more of: (a) serum monoclonal protein (M-protein) ≥0.5 g/dL ≥5 g/L); (b) urine M-protein 200 mg/24 hr; and/or (c) serum free light chain (SFLC) assay: Involved SFLCs ≥10 mg/dL (≥100 mg/L) and an abnormal SFLC ratio (<0.26 or >1.65).

In some aspects, the patient has a total hemoglobin ≥8 g/dL and serum creatinine ≤2.0 mg/dL and creatinine clearance ≥30 mL/min (calculated or per 24-hr urine collection). In some aspects, the subject does not have primary or secondary plasma cell leukemia as defined by an absolute plasma cell count exceeding 2000/μL or 20% of the peripheral blood white cells

In any of the methods, uses, and compositions for use described herein, the anti-TIGIT antagonist antibody (e.g., an anti-TIGIT antagonist antibody as disclosed herein, e.g., tiragolumab), or medicament thereof, and anti-CD20 antibody (e.g., rituximab), or medicament thereof, is for treating a subject having a hematologic cancer. In some aspects, the hematologic cancer is a lymphoma. In some aspects the lymphoma is a non-Hodgkin's lymphoma (NHL). In some aspects, the NHL is a relapsed or refractory diffuse large B cell lymphoma (DLBCL). In other aspects, the NHL is a relapsed or refractory follicular lymphoma (FL). In some aspects, the subject has a history of histologically-documented DLBCL or transformed FL and have relapsed after or failed to respond to at least two prior systemic treatment regimens (e.g., including at least one prior regimen containing anthracycline, and at least one containing an anti-CD20-directed therapy) and for which no suitable therapy of curative intent or higher priority exists (e.g., standard chemotherapy, autologous SCT). In some aspects the subject has at least one bi-dimensionally measurable lesion (>1.5 cm in its largest dimension by computerized tomography [CT] scan). In other aspects, the subject does not have a current or a history of CNS lymphoma. In some aspects, the subject has a total hemoglobin ≥9 g/dL and a serum creatinine ≤ULN or estimated creatinine CL ≥60 mL/min.

In some aspects in any of the methods, uses, or compositions for use described herein, the subject has an Eastern Cooperative Oncology Group (ECOG) Performance Status (PS) of 0 or 1 and a life expectancy of 12 weeks. In some aspects, the subject has an AST and ALT ≤3× upper limit of normal (ULN), and total serum bilirubin of ≤1.5×ULN, and an alkaline phosphatase s 2.5×ULN. In some aspects, the subject has a platelet count ≥75,000/μL and ANC 1000/μL. In some aspects, in any of the methods, uses, or compositions for use described herein, the subject does not have an active Epstein-Barr virus (EBV) infection or a known or suspected chronic active EBV infection. In some aspects, the subject is negative for EBV IgM and/or negative by EBV PCR. In some aspects, the subject is negative for EBV IgM and/or negative by EBV PCR and is positive for EBV IgG and/or positive for Epstein-Barr nuclear antigen (EBNA). In other aspects, the subject is negative for EBV IgG and/or negative for EBNA. In some aspects, patients have not used any chemotherapy, monoclonal antibody, radioimmunoconjugate, antibody-drug conjugate, hormonal therapy, and/or radiotherapy within 4 weeks prior to administration of the anti-TIGIT antagonist antibody, anti-CD38 antibody, and/or anti-CD20 antibody.

In some aspects, in any of the methods, uses, or compositions for use described herein, administration of the anti-TIGIT antagonist antibody and the anti-CD38 antibody or anti-CD20 antibody results in a clinical response. In some aspects, the clinical response is an increase in the objective response rate (ORR), the duration of objective response (DOR), and/or the progression-free survival (PFS) of the subject compared to a reference ORR, DOR, and/or PFS. In some aspects, the reference ORR, DOR, and/or PFS may be from a reference population. In some aspects, the reference population may, for example, be (a) a population of patients who have received the anti-TIGIT antagonist antibody, without the anti-CD38 antibody or anti-CD20 antibody, or (b) a population of patients who have received the anti-CD38 antibody or anti-CD20 antibody, without the anti-TIGIT antagonist antibody. In some aspects, the ORR, DOR, and PFS are assessed using the International Myeloma Working Group Uniform Response (IMWG) criteria. In other aspects, the ORR, DOR, and PFS are assessed using the Lugano Response Criteria for Malignant Lymphoma (Lugano classification). In some aspects, the ORR may be assessed during screening, during each of cycles 1-21, and at discontinuation of treatment.

The therapeutic methods and uses of the invention described herein, include, in another aspect, administering to a subject having a cancer (e.g., a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL))) an effective amount of an anti-TIGIT antagonist antibody (e.g., an anti-TIGIT antagonist antibody described herein, e.g., tiragolumab) in a dosing regimen comprising one or more dosing cycles, wherein the anti-TIGIT antagonist antibody is administered once every three weeks, thereby treating the subject. In some aspects, the anti-TIGIT antagonist antibody (e.g., an anti-TIGIT antagonist antibody described herein, e.g., tiragolumab) is administered as a monotherapy.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In any of the methods and uses of the invention, the anti-TIGIT antagonist antibody (e.g., an anti-TIGIT antagonist antibody as disclosed herein, e.g., tiragolumab) may be administered in a dosing regimen that includes one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 9 dosing cycles. In other aspects, the dosing regimen includes at least 12 dosing cycles. In other aspects, the dosing regimen includes at least 16 dosing cycles. In some aspects, the dosing cycles of the anti-TIGIT antagonist antibody (e.g., an anti-TIGIT antagonist antibody as disclosed herein, e.g., tiragolumab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-TIGIT antagonist antibody (e.g., an anti-TIGIT antagonist antibody as disclosed herein, e.g., tiragolumab) is administered on about day 1 (e.g., day 1±1 day) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab) is administered intravenously at a fixed dose of about 600 mg on day 2 of each 21 day cycle (i.e., at a fixed dose of about 600 mg every three weeks).

In some aspects, following administration of the anti-TIGIT antagonist antibody, the method includes an observation period. In some aspects, the observation period is between about 30 minutes to about 60 minutes in length. In aspects in which the observation period is about 60 minutes in length, the method may include recording the subject's vital signs (e.g., pulse rate, respiratory rate, blood pressure, and temperature) at about 30±10 minutes after administration of the anti-TIGIT antagonist antibody during the observation period. In aspects in which the observation period is about 30 minutes in length, the method may include recording the subject's vital signs (e.g., pulse rate, respiratory rate, blood pressure, and temperature) at about 15±10 minutes after administration of the anti-TIGIT antagonist antibody during the observation period.

In another aspect, the invention provides a method of treating a subject having a relapsed or refractory MM by administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle. In some aspects, the dosing regimen comprises at least 12 dosing cycles. In other aspects, the dosing regimen comprises at least 16 dosing cycles. In some aspects, tiragolumab is administered as a monotherapy.

In another aspect, the invention provides a method of treating a subject having a relapsed or refractory NHL by administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle. In some aspects, the dosing regimen comprises at least 12 dosing cycles. In other aspects, the dosing regimen comprises at least 16 dosing cycles. In some aspects, tiragolumab is administered as a monotherapy.

In another aspect, the invention provides an anti-TIGIT antagonist antibody (e.g., an anti-TIGIT antagonist antibody disclosed herein, e.g., tiragolumab) for use in a method of treating a subject having a cancer (e.g., a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL))), wherein the method comprises administering to the subject an effective amount of an anti-TIGIT antagonist antibody (e.g., an anti-TIGIT antagonist antibody described herein, e.g., tiragolumab) in a dosing regimen comprising one or more dosing cycles, wherein the anti-TIGIT antagonist antibody is administered once every three weeks. In some aspects, the anti-TIGIT antagonist antibody (e.g., an anti-TIGIT antagonist antibody described herein, e.g., tiragolumab) is to be administered as a monotherapy.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In any of the uses of the invention, the anti-TIGIT antagonist antibody (e.g., an anti-TIGIT antagonist antibody as disclosed herein, e.g., tiragolumab) is to be administered in a dosing regimen that includes one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 9 dosing cycles. In other aspects, the dosing regimen includes at least 12 dosing cycles. In other aspects, the dosing regimen includes at least 16 dosing cycles. In some aspects, the dosing cycles of the anti-TIGIT antagonist antibody (e.g., an anti-TIGIT antagonist antibody as disclosed herein, e.g., tiragolumab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-TIGIT antagonist antibody (e.g., an anti-TIGIT antagonist antibody as disclosed herein, e.g., tiragolumab) is to be administered on about day 1 (e.g., day 1±1 day) of each dosing cycle. For example, the anti-TIGIT antagonist antibody (e.g., an anti-TIGIT antagonist antibody as disclosed herein, e.g., tiragolumab) is to be administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab) is to be administered intravenously at a fixed dose of about 600 mg on day 2 of each 21 day cycle (i.e., at a fixed dose of about 600 mg every three weeks).

In some aspects, following administration of the anti-TIGIT antagonist antibody, the method includes an observation period. In some aspects, the observation period is between about 30 minutes to about 60 minutes in length. In aspects in which the observation period is about 60 minutes in length, the method may include recording the subject's vital signs (e.g., pulse rate, respiratory rate, blood pressure, and temperature) at about 30±10 minutes after administration of the anti-TIGIT antagonist during the observation period. In aspects in which the observation period is about 30 minutes in length, the method may include recording the subject's vital signs (e.g., pulse rate, respiratory rate, blood pressure, and temperature) at about 15±10 minutes after administration of the anti-TIGIT antagonist antibody during the observation period.

In another aspect, the invention provides tiragolumab for use in a method of treating a subject having a relapsed or refractory MM, wherein the method comprises administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle. In some aspects, the dosing regimen comprises at least 12 dosing cycles. In other aspects, the dosing regimen comprises at least 16 dosing cycles. In some aspects, tiragolumab is to be administered as a monotherapy.

In another aspect, the invention provides tiragolumab for use in a method of treating a subject having a relapsed or refractory NHL, wherein the method comprises administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle. In some aspects, the dosing regimen comprises at least 12 dosing cycles. In other aspects, the dosing regimen comprises at least 16 dosing cycles. In some aspects, tiragolumab is to be administered as a monotherapy.

In another aspect, the invention provides uses of an effective amount of an anti-TIGIT antagonist antibody (e.g., an anti-TIGIT antagonist antibody disclosed herein, e.g., tiragolumab) in the manufacture or preparation of a medicament for use in a method of treating a subject having a cancer (e.g., a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL))), wherein the method comprises administering to the subject an effective amount of the medicament comprising the anti-TIGIT antagonist antibody in a dosing regimen comprising one or more dosing cycles, wherein the medicament comprising the anti-TIGIT antagonist antibody is administered once every three weeks. In some aspects, the medicament comprising the anti-TIGIT antagonist antibody (e.g., an anti-TIGIT antagonist antibody described herein, e.g., tiragolumab) is to be administered as a monotherapy.

In some aspects, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some aspects, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some aspects, the effective amount of the anti-TIGIT antagonist antibody (e.g., an anti-TIGIT antagonist antibody as disclosed herein, e.g., tiragolumab) is a fixed dose of about 600 mg every three weeks. In some aspects, effective amount of the anti-TIGIT antagonist antibody (e.g., an anti-TIGIT antagonist antibody as disclosed herein, e.g., tiragolumab) is a fixed dose of 600 mg.

In any of the uses of the invention, the medicament comprising the anti-TIGIT antagonist antibody (e.g., an anti-TIGIT antagonist antibody as disclosed herein, e.g., tiragolumab) is to be administered in a dosing regimen that includes one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In other aspects, the dosing regimen includes at least 9 dosing cycles. In other aspects, the dosing regimen includes at least 12 dosing cycles. In other aspects, the dosing regimen includes at least 16 dosing cycles. In some aspects, the dosing cycles of the medicament comprising the anti-TIGIT antagonist antibody (e.g., an anti-TIGIT antagonist antibody as disclosed herein, e.g., tiragolumab) continue until there is a loss of clinical benefit (e.g., confirmed disease progression, drug resistance, death, or unacceptable toxicity). In some aspects, the length of each dosing cycle is about 18 to 24 days (e.g., 15 days, 16 days, 17 days, 18 days, 19 days, 20 days, 21 days, 22 days, 23 days, or 24 days). In some aspects, the length of each dosing cycle is about 21 days.

In some aspects, the anti-TIGIT antagonist antibody (e.g., an anti-TIGIT antagonist antibody as disclosed herein, e.g., tiragolumab), or medicament thereof, is to be administered on about day 1 (e.g., day 1±1 day) of each dosing cycle. For example, the anti-TIGIT antagonist antibody (e.g., an anti-TIGIT antagonist antibody as disclosed herein, e.g., tiragolumab), or medicament thereof, is to be administered intravenously at a fixed dose of about 600 mg on day 1 of each 21 day cycle (i.e., at a fixed dose of about 600 mg every three weeks). In another aspect, the anti-TIGIT antagonist antibody (e.g., an anti-TIGIT antagonist antibody as disclosed herein, e.g., tiragolumab), or medicament thereof, is to be administered intravenously at a fixed dose of about 600 mg on day 2 of each 21 day cycle (i.e., at a fixed dose of about 600 mg every three weeks).

In some aspects, following administration of the anti-TIGIT antagonist antibody, the method includes an observation period. In some aspects, the observation period is between about 30 minutes to about 60 minutes in length. In aspects in which the observation period is about 60 minutes in length, the method may include recording the subject's vital signs (e.g., pulse rate, respiratory rate, blood pressure, and temperature) at about 30±10 minutes after administration of the anti-TIGIT antagonist antibody during the observation period. In aspects in which the observation period is about 30 minutes in length, the method may include recording the subject's vital signs (e.g., pulse rate, respiratory rate, blood pressure, and temperature) at about 15±10 minutes after administration of the anti-TIGIT antagonist antibody during the observation period.

In another aspect, the invention provides tiragolumab in the manufacture or preparation of a medicament for use in a method of treating a subject having relapsed or refractory MM, wherein the method comprises administering to the subject 600 mg of the medicament comprising the anti-TIGIT antagonist antibody in a dosing regimen comprising one or more dosing cycles, wherein the medicament comprising the anti-TIGIT antagonist antibody is administered once every three weeks. In some aspects, the dosing regimen comprises at least 12 dosing cycles. In other aspects, the dosing regimen comprises at least 16 dosing cycles. In some aspects, the medicament comprising tiragolumab is to be administered as a monotherapy.

In another aspect, the invention provides tiragolumab in the manufacture or preparation of a medicament for use in a method of treating a subject having relapsed or refractory NHL, wherein the method comprises administering to the subject 600 mg of the medicament comprising the anti-TIGIT antagonist antibody in a dosing regimen comprising one or more dosing cycles, wherein the medicament comprising the anti-TIGIT antagonist antibody is administered once every three weeks. In some aspects, the dosing regimen comprises at least 12 dosing cycles. In other aspects, the dosing regimen comprises at least 16 dosing cycles. In some aspects, the medicament comprising tiragolumab is to be administered as a monotherapy.

In any of the methods, uses, and compositions for use described herein, the subject has an infusion-related reaction to the anti-TIGIT antagonist antibody (e.g., an anti-TIGIT antagonist antibody as disclosed herein, e.g., tiragolumab). In such aspects, the method further includes administering to the subject an antihistamine (e.g., diphenhydramine) and/or an antipyretic (e.g., acetaminophen) prior to a subsequent administration of the anti-TIGIT antagonist antibody (e.g., an anti-TIGIT antagonist antibody as disclosed herein, e.g., tiragolumab).

In any of the methods, uses, and compositions for use described herein, the anti-TIGIT antagonist antibody (e.g., an anti-TIGIT antagonist antibody as disclosed herein, e.g., tiragolumab), or medicament thereof, is for treating a subject having a hematologic cancer.

In some aspects, the hematologic cancer is a myeloma. In some aspects, the myeloma is a multiple myeloma (MM). In some aspects, the MM is a relapsed or refractory MM. In some aspects, the subject has a recurrent or relapsed MM for which no established therapy for MM is appropriate and available or be intolerant to those established therapies. In some aspects, the subject has received at least three prior therapeutic regimens (e.g., including at least one prior regimen containing a proteasome inhibitor, at least one containing an immunomodulatory drug, and at least one containing an anti-CD38 antibody). In some aspects, measurable disease is defined as a subject having one or more of: (a) serum monoclonal protein (M-protein) ≥0.5 g/dL ≥5 g/L); (b) urine M-protein ≥200 mg/24 hr; and/or (c) serum free light chain (SFLC) assay: Involved SFLCs ≥10 mg/dL (≥100 mg/L) and an abnormal SFLC ratio (<0.26 or >1.65).

In some aspects, the patient has a total hemoglobin ≥8 g/dL and serum creatinine ≤2.0 mg/dL and creatinine clearance ≥30 mL/min (calculated or per 24-hr urine collection). In some aspects, the subject does not have primary or secondary plasma cell leukemia as defined by an absolute plasma cell count exceeding 2000/μL or 20% of the peripheral blood white cells

In some aspects, the hematologic cancer is a lymphoma. In some aspects, the lymphoma is a non-Hodgkin's lymphoma (NHL). In some aspects, the NHL is a relapsed or refractory diffuse large B cell lymphoma (DLBCL). In other aspects, the NHL is a relapsed or refractory follicular lymphoma (FL). In some aspects, the subject has a history of histologically-documented DLBCL or transformed FL and have relapsed after or failed to respond to at least two prior systemic treatment regimens (e.g., including at least one prior regimen containing anthracycline, and at least one containing an anti-CD20-directed therapy) and for which no suitable therapy of curative intent or higher priority exists (e.g., standard chemotherapy, autologous SCT). In some aspects the subject has at least one bi-dimensionally measurable lesion (>1.5 cm in its largest dimension by computerized tomography [CT] scan). In other aspects, the subject does not have a current or a history of CNS lymphoma. In some aspects, the subject has a total hemoglobin 9 g/dL and a serum creatinine ≤ULN or estimated creatinine CL ≥60 mL/min.

In some aspects in any of the methods, uses, or compositions for use described herein, the subject has an Eastern Cooperative Oncology Group (ECOG) Performance Status (PS) of 0 or 1 and a life expectancy of 12 weeks. In some aspects, the subject has an AST and ALT ≤3×ULN, a total serum bilirubin of ≤1.5×ULN, and alkaline phosphatase of s 2.5×ULN. In some aspects, the subject has a platelet count ≥75,000/μL and ANC 1000/μL. In some aspects, in any of the methods, uses, or compositions for use described herein, the subject does not have an active Epstein-Barr virus (EBV) infection or a known or suspected chronic active EBV infection. In some aspects, the subject is negative for EBV IgM and/or negative by EBV PCR. In some aspects, the subject is negative for EBV IgM and/or negative by EBV PCR and is positive for EBV IgG and/or positive for Epstein-Barr nuclear antigen (EBNA). In other aspects, the subject is negative for EBV IgG and/or negative for EBNA. In some aspects, patients have not used any chemotherapy, monoclonal antibody, radioimmunoconjugate, antibody-drug conjugate, hormonal therapy, and/or radiotherapy within 4 weeks prior to administration of the anti-TIGIT antagonist antibody, anti-CD38 antibody, and/or anti-CD20 antibody.

In some aspects, in any of the methods, uses, or compositions for use described herein, administration of the anti-TIGIT antagonist antibody results in a clinical response. In some aspects, the clinical response is an increase in the objective response rate (ORR), the duration of objective response (DOR), and/or the progression-free survival (PFS) of the subject compared to a reference ORR, DOR, and/or PFS. In some aspects, the reference ORR, DOR, and/or PFS may be from a reference population. In some aspects, the reference population may, for example, be a population of patients who have received a therapy other than an anti-TIGIT antagonist antibody (e.g., an anti-CD38 antibody (e.g., daratumumab) or anti-CD20 antibody (e.g., rituximab)). In some aspects, the ORR, DOR, and PFS are assessed using the International Myeloma Working Group Uniform Response (IMWG) criteria. In other aspects, the ORR, DOR, and PFS are assessed using the Lugano Response Criteria for Malignant Lymphoma (Lugano classification). In some aspects, the ORR may be assessed during screening, during each of cycles 1-21, and at discontinuation of treatment.

### IV. Exemplary Antibodies for Use in the Methods and Uses of the Invention

Exemplary anti-TIGIT antagonist antibodies, anti-CD20 antibodies, and anti-CD38 antibodies useful for treating a subject (e.g., a human) having cancer (e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)) in accordance with the methods, uses, and compositions for use of the invention are described herein.

### A. Exemplary Anti-TIGIT Antagonist Antibodies

The invention provides anti-TIGIT antagonist antibodies useful for treating cancer (e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)) in a subject (e.g., a human).

In certain aspects, the anti-TIGIT antagonist antibodies includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some aspects, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-10. In some aspects, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some aspects, any of the above anti-TIGIT antagonist antibodies includes (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6).

In some aspects, the anti-TIGIT antagonist antibody further comprises at least one, two, three, or four of the following heavy chain variable region FRs: an FR-H1 comprising the amino acid sequence of X₁VQLQQSGPGLVKPSQTLSLTCAISGDSVS (SEQ ID NO: 11), wherein X₁ is Q or E; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some aspects, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In another aspect, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some aspects, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In some aspects, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, EVQLQQSGPGLVKPSQTLSLTCAISGDSVSSNSAAWNWIRQSPSRGLEWLGKTYYRFKWYSDYAVS VKGRITINPDTSKNQFSLQLNSVTPEDTAVFYCTRESTTYDLLAGPFDYWGQGTLVTVSS (SEQ ID NO: 17) or QVQLQQSGPGLVKPSQTLSLTCAISGDSVSSNSAAWNWIRQSPSRGLEWLGKTYYRFKWYSDYAVS VKGRITINPDTSKNQFSLQLNSVTPEDTAVFYCTRESTTYDLLAGPFDYWGQGTLVTVSS (SEQ ID NO: 18) and/or a VL domain comprising an amino acid sequence having at least 90% sequence identity (e.g., at least 91%, 92%, 93%, 94%, 95%, 96%, 97%, 98%, or 99% sequence identity) to, or the sequence of, DIVMTQSPDSLAVSLGERATINCKSSQTVLYSSNNKKYLAWYQQKPGQPPNLLIYWASTRESGVPDR FSGSGSGTDFTLTISSLQAEDVAVYYCQQYYSTPFTFGPGTKVEIK (SEQ ID NO: 19). In some aspects, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some aspects, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19.

In another aspect, an anti-TIGIT antagonist antibody is provided, wherein the antibody comprises a VH as in any of the aspects provided above, and a VL as in any of the aspects provided above, wherein one or both of the variable domain sequences include post-translational modifications.

In some aspects, any one of the anti-TIGIT antagonist antibodies described above is capable of binding to rabbit TIGIT, in addition to human TIGIT. In some aspects, any one of the anti-TIGIT antagonist antibodies described above is capable of binding to both human TIGIT and cynomolgus monkey (cyno) TIGIT. In some aspects, any one of the anti-TIGIT antagonist antibodies described above is capable of binding to human TIGIT, cyno TIGIT, and rabbit TIGIT. In some aspects, any one of the anti-TIGIT antagonist antibodies described above is capable of binding to human TIGIT, cyno TIGIT, and rabbit TIGIT, but not murine TIGIT.

In some aspects, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some aspects, the anti-TIGIT antagonist antibody specifically binds TIGIT and inhibit or block TIGIT interaction with poliovirus receptor (PVR) (e.g., the antagonist antibody inhibits intracellular signaling mediated by TIGIT binding to PVR). In some aspects, the antagonist antibody inhibits or blocks binding of human TIGIT to human PVR with an IC50 value of 10 nM or lower (e.g., 1 nM to about 10 nM). In some aspects, the antagonist antibody inhibits or blocks binding of cyno TIGIT to cyno PVR with an IC50 value of 50 nM or lower (e.g., 1 nM to about 50 nM, e.g., 1 nM to about 5 nM).

In some aspects, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

In certain aspects, the anti-TIGIT antagonist antibody is tiragolumab (CAS Registry Number: 1918185-84-8). Tiragolumab (Genentech) is also known as MTIG7192A. Examples of anti-TIGIT antibodies useful for the methods of this invention and methods for making thereof are described in PCT Pub. No: WO 2017/053748, herein incorporated by reference. The anti-TIGIT antagonist antibodies (e.g., tiragolumab) useful in this invention, including compositions containing such antibodies, may be used in combination with an anti-CD38 antibody or an anti-CD20 antibody to treat a hematologic cancer (e.g., a myeloma (e.g., MM, e.g., a relapsed or refractory MM) or a lymphoma (e.g., a NHL, e.g., a relapsed or refractory DLBCL or a relapsed or refractory FL)).

An anti-TIGIT antagonist antibody according to any of the above aspects may be a monoclonal antibody, comprising a chimeric, humanized, or human antibody. In one aspect, an anti-TIGIT antagonist antibody is an antibody fragment, for example, a Fv, Fab, Fab′, scFv, diabody, or F(ab′)₂ fragment. In another aspect, the antibody is a full-length antibody, e.g., an intact IgG antibody (e.g., an intact IgG1 antibody) or other antibody class or isotype as defined herein.

In a further aspect, an anti-TIGIT antagonist antibody according to any of the above aspects may incorporate any of the features, singly or in combination, as described in Sections 1-6 below.

B. Exemplary Anti-CD38 Antibodies

Provided herein are methods for treating cancer (e.g., a hematologic cancer, e.g., a myeloma (e.g., MM, e.g., a relapsed or refractory MM)) in a subject (e.g., a human) in a subject comprising administering to the subject an effective amount of an anti-CD38 antibody.

In certain aspects, the anti-CD38 antibodies includes at least one, two, three, four, five, or six HVRs selected from: (a) an HVR-H1 comprising the amino acid sequence of SFAMS (SEQ ID NO: 20); (b) an HVR-H2 comprising the amino acid sequence of AISGSGGGTYYADSVKG (SEQ ID NO: 21); (c) an HVR-H3 comprising the amino acid sequence of DKILWFGEPVFDY (SEQ ID NO: 22); (d) an HVR-L1 comprising the amino acid sequence of RASQSVSSYLA (SEQ ID NO: 23), (e) an HVR-L2 comprising the amino acid sequence of DASNRAT (SEQ ID NO: 24); and/or (f) an HVR-L3 comprising the amino acid sequence of QQRSNWPPTF (SEQ ID NO: 25), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 20-25.

In some aspects, any of the above anti-CD38 antibodies includes (a) an HVR-H1 comprising the amino acid sequence of SFAMS (SEQ ID NO: 20); (b) an HVR-H2 comprising the amino acid sequence of AISGSGGGTYYADSVKG (SEQ ID NO: 21); (c) an HVR-H3 comprising the amino acid sequence of DKILWFGEPVFDY (SEQ ID NO: 22); (d) an HVR-L1 comprising the amino acid sequence of RASQSVSSYLA (SEQ ID NO: 23); (e) an HVR-L2 comprising the amino acid sequence of DASNRAT (SEQ ID NO: 24); and (f) an HVR-L3 comprising the amino acid sequence of QQRSNWPPTF (SEQ ID NO: 25).

In some aspects, the anti-CD38 antibody further comprises at least one, two, three, or four of the following light chain variable region framework regions (FRs): an FR-L1 comprising the amino acid sequence of EIVLTQSPATLSLSPGERATLSC (SEQ ID NO: 26); an FR-L2 comprising the amino acid sequence of WYQQKPGQAPRLLIY (SEQ ID NO: 27); an FR-L3 comprising the amino acid sequence of GIPARFSGSGSGTDFTLTISSLEPEDFAVYYC (SEQ ID NO: 28); and/or an FR-L4 comprising the amino acid sequence of GQGTKVEIK (SEQ ID NO: 29), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 26-29. In some aspects, for example, the antibody further comprises an FR-L1 comprising the amino acid sequence of EIVLTQSPATLSLSPGERATLSC (SEQ ID NO: 26); an FR-L2 comprising the amino acid sequence of WYQQKPGQAPRLLIY (SEQ ID NO: 27); an FR-L3 comprising the amino acid sequence of GIPARFSGSGSGTDFTLTISSLEPEDFAVYYC (SEQ ID NO: 28); and an FR-L4 comprising the amino acid sequence of GQGTKVEIK (SEQ ID NO: 29).

In some aspects, the anti-CD38 antibody further comprises at least one, two, three, or four of the following heavy chain variable region FRs: an FR-H1 comprising the amino acid sequence of EVQLLESGGGLVQPGGSLRLSCAVSGFTFN (SEQ ID NO: 30); an FR-H2 comprising the amino acid sequence of WVRQAPGKGLEWVS (SEQ ID NO: 31); an FR-H3 comprising the amino acid sequence of RFTISRDNSKNTLYLQMNSLRAEDTAVYFCAK (SEQ ID NO: 32); and/or an FR-H4 comprising the amino acid sequence of WGQGTLVTVSS (SEQ ID NO: 33), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 30-33. In some aspects, the anti-CD38 antibody includes an FR-H1 comprising the amino acid sequence of EVQLLESGGGLVQPGGSLRLSCAVSGFTFN (SEQ ID NO: 30); an FR-H2 comprising the amino acid sequence of WVRQAPGKGLEWVS (SEQ ID NO: 31); an FR-H3 comprising the amino acid sequence of RFTISRDNSKNTLYLQMNSLRAEDTAVYFCAK (SEQ ID NO: 32); and an FR-H4 comprising the amino acid sequence of WGQGTLVTVSS (SEQ ID NO: 33).

In some aspects, the anti-CD38 antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of EVQLLESGGGLVQPGGSLRLSCAVSGFTFNSFAMSWVRQAPGKGLEWVSAISGSGGGTYYADSVK GRFTISRDNSKNTLYLQMNSLRAEDTAVYFCAKDKILWFGEPVFDYWGQGTLVTVSS (SEQ ID NO: 34) and/or a VL domain comprising an amino acid sequence having at least 90% sequence identity (e.g., at least 91%, 92%, 93%, 94%, 95%, 96%, 97%, 98%, or 99% sequence identity) to, or the sequence of EIVLTQSPATLSLSPGERATLSCRASQSVSSYLAWYQQKPGQAPRLLIYDASNRATGIP ARFSGSGSGTDFTLTISSLEPEDFAVYYCQQRSNWPPTFGQGTKVEIK (SEQ ID NO: 35).

In another aspect, an anti-CD38 antibody is provided, wherein the antibody comprises a VH as in any of the aspects provided above, and a VL as in any of the aspects provided above, wherein one or both of the variable domain sequences include post-translational modifications.

In some aspects, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. In some aspects, an anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹ M to 10⁻¹³ M). In certain aspects, the anti-CD38 antibody may bind to both human CD38 and chimpanzee CD38.

In some aspects, the methods or uses described herein may include using or administering an isolated anti-CD38 antibody that competes for binding to CD38 with any of the anti-CD38 antibodies described above. For example, the method may include administering an isolated anti-CD38 antibody that competes for binding to CD38 with an anti-CD38 antibody having the following six HVRs: (a) an HVR-H1 comprising the amino acid sequence of SFAMS (SEQ ID NO: 20); (b) an HVR-H2 comprising the amino acid sequence of AISGSGGGTYYADSVKG (SEQ ID NO:21); (c) an HVR-H3 comprising the amino acid sequence of DKILWFGEPVFDY (SEQ ID NO: 22); (d) an HVR-L1 comprising the amino acid sequence of RASQSVSSYLA (SEQ ID NO:23), (e) an HVR-L2 comprising the amino acid sequence of DASNRAT (SEQ ID NO: 24); and (f) an HVR-L3 comprising the amino acid sequence of QQRSNWPPTF (SEQ ID NO: 25). The methods described herein may also include administering an isolated anti-CD38 antibody that binds to the same epitope as an anti-CD38 antibody described above.

In certain aspects, the anti-CD38 antibody is daratumumab (DARZALEX®). In other aspects, the anti-CD38 antibody is MOR202 or isatuximab (SAR-650984). Examples of anti-CD38 antibodies useful for the methods of this invention and methods for making thereof are described in U.S. Pat. Nos. 7,829,673; 8,263,746; and 8,153,765; and U.S. Pub. No: 20160067205 A1. The anti-CD38 antibodies (e.g., daratumumab) useful in this invention, including compositions containing such antibodies, may be used in combination with an anti-TIGIT antagonist antibody to treat a hematologic cancer (e.g., a myeloma (e.g., MM, e.g., a relapsed or refractory MM)).

An anti-CD38 antibody according to any of the above aspects may be a monoclonal antibody, comprising a chimeric, humanized, or human antibody. In one aspect, an anti-CD38 antibody is an antibody fragment, for example, a Fv, Fab, Fab′, scFv, diabody, or F(ab′)₂ fragment. In another aspect, the antibody is a full-length antibody, e.g., an intact IgG antibody (e.g., an intact IgG1 antibody) or other antibody class or isotype as defined herein.

In a further aspect, an anti-CD38 antibody according to any of the above aspects may incorporate any of the features, singly or in combination, as described in Sections 1-6 below.

C. Exemplary Anti-CD20 Antibodies

Provided herein are methods for treating cancer (e.g., a hematologic cancer, e.g., a lymphoma (e.g., a NHL, e.g., a relapsed or refractory DLBCL or a relapsed or refractory FL)) in a subject (e.g., a human) in a subject comprising administering to the subject an effective amount of an anti-CD20 antibody.

In certain aspects, the anti-CD20 antibodies includes at least one, two, three, four, five, or six HVRs selected from: (a) an HVR-H1 comprising the amino acid sequence of SYNMH (SEQ ID NO: 36); (b) an HVR-H2 comprising the amino acid sequence of AIYPGNGDTSYNQKFKG (SEQ ID NO: 37); (c) an HVR-H3 comprising the amino acid sequence of STYYGGDWYFNV (SEQ ID NO: 38); (d) an HVR-L1 comprising the amino acid sequence of RASSSVSYIH (SEQ ID NO: 39), (e) an HVR-L2 comprising the amino acid sequence of ATSNLAS (SEQ ID NO: 40); and/or (f) an HVR-L3 comprising the amino acid sequence of QQWTSNPPT (SEQ ID NO: 41), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 36-41.

In some aspects, any of the above anti-CD20 antibodies includes (a) an HVR-H1 comprising the amino acid sequence of SYNMH (SEQ ID NO: 36); (b) an HVR-H2 comprising the amino acid sequence of AIYPGNGDTSYNQKFKG (SEQ ID NO: 37); (c) an HVR-H3 comprising the amino acid sequence of STYYGGDWYFNV (SEQ ID NO: 38); (d) an HVR-L1 comprising the amino acid sequence of RASSSVSYIH (SEQ ID NO: 39); (e) an HVR-L2 comprising the amino acid sequence of ATSNLAS (SEQ ID NO: 40); and (f) an HVR-L3 comprising the amino acid sequence of QQWTSNPPT (SEQ ID NO: 41). In some aspects, the anti-CD20 antibody further comprises at least one, two, three, or four of the following light chain variable region framework regions (FRs): an FR-L1 comprising the amino acid sequence of QIVLSQSPAILSASPGEKVTMTC (SEQ ID NO: 42); an FR-L2 comprising the amino acid sequence of WFQQKPGSSPKPWIY (SEQ ID NO: 43); an FR-L3 comprising the amino acid sequence of GVPVRFSGSGSGTSYSLTISRVEAEDAATYYC (SEQ ID NO: 44); and/or an FR-L4 comprising the amino acid sequence of FGGGTKLEIK (SEQ ID NO: 45), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 42-45. In some aspects, for example, the antibody further comprises an FR-L1 comprising the amino acid sequence of QIVLSQSPAILSASPGEKVTMTC (SEQ ID NO: 42); an FR-L2 comprising the amino acid sequence of WFQQKPGSSPKPWIY (SEQ ID NO: 43); an FR-L3 comprising the amino acid sequence of GVPVRFSGSGSGTSYSLTISRVEAEDAATYYC (SEQ ID NO: 44); and an FR-L4 comprising the amino acid sequence of FGGGTKLEIK (SEQ ID NO: 45).

In some aspects, the anti-CD20 antibody further comprises at least one, two, three, or four of the following heavy chain variable region FRs: an FR-H1 comprising the amino acid sequence of QVQLQQPGAELVKPGASVKMSCKASGYTFT (SEQ ID NO: 46); an FR-H2 comprising the amino acid sequence of WVKQTPGRGLEWIG (SEQ ID NO: 47); an FR-H3 comprising the amino acid sequence of KATLTADKSSSTAYMQLSSLTSEDSAVYYCAR (SEQ ID NO: 48); and/or an FR-H4 comprising the amino acid sequence of WGAGTTVTVS (SEQ ID NO: 49), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 46-49. In some aspects, the anti-CD20 antibody includes an FR-H1 comprising the amino acid sequence of QVQLQQPGAELVKPGASVKMSCKASGYTFT (SEQ ID NO: 46); an FR-H2 comprising the amino acid sequence of WVKQTPGRGLEWIG (SEQ ID NO: 47); an FR-H3 comprising the amino acid sequence of KATLTADKSSSTAYMQLSSLTSEDSAVYYCAR (SEQ ID NO: 48); and an FR-H4 comprising the amino acid sequence of WGAGTTVTVS (SEQ ID NO: 49).

In some aspects, the anti-CD20 antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of QVQLQQPGAELVKPGASVKMSCKASGYTFTSYNMHWVKQTPGRGLEWIGAIYPGNGDTSYNQKFK GKATLTADKSSSTAYMQLSSLTSEDSAVYYCARSTYYGGDWYFNVWGAGTTVTVS (SEQ ID NO: 50) and/or a VL domain comprising an amino acid sequence having at least 90% sequence identity (e.g., at least 91%, 92%, 93%, 94%, 95%, 96%, 97%, 98%, or 99% sequence identity) to, or the sequence of QIVLSQSPAILSASPGEKVTMTCRASSSVSYIHWFQQKPGSSPKPWIYATSNLASGVPVRFSGSGSG TSYSLTISRVEAEDAATYYCQQWTSNPPTFGGGTKLEIK (SEQ ID NO: 51).

In another aspect, an anti-CD20 antibody is provided, wherein the antibody comprises a VH as in any of the aspects provided above, and a VL as in any of the aspects provided above, wherein one or both of the variable domain sequences include post-translational modifications.

In certain aspects, an anti-CD20 antibody may bind to CD20 on the surface of a malignant B cell and mediate B cell lysis through the activation of complement-dependent lysis, antibody-dependent cellular cytotoxicity (ADCC), and apoptosis mediated by Fc cross-linking, leading to the depletion of circulating B lymphocytes. In certain aspects, an anti-CD20 antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹ M to 10⁻¹³ M). In certain aspects, an anti-CD20 antibody that binds to CD20 has a K_{D} of <10 nM. In certain aspects, the binding is at a K_{D} of <7.5 nM, <5 nM, between 1-5 nM, or <1 nM. In certain aspects, the anti-CD20 antibody may bind to both human CD20 and cyno CD20.

In some aspects, the methods or uses described herein may include using or administering an isolated anti-CD20 antibody that competes for binding to CD20 with any of the anti-CD20 antibodies described above. For example, the method may include administering an isolated anti-CD20 antibody that competes for binding to CD20 with an anti-CD20 antibody having the following six HVRs: (a) an HVR-H1 comprising the amino acid sequence of SYNMH (SEQ ID NO: 36); (b) an HVR-H2 comprising the amino acid sequence of AIYPGNGDTSYNQKFKG (SEQ ID NO: 37); (c) an HVR-H3 comprising the amino acid sequence of STYYGGDWYFNV (SEQ ID NO: 38); (d) an HVR-L1 comprising the amino acid sequence of RASSSVSYIH (SEQ ID NO: 39), (e) an HVR-L2 comprising the amino acid sequence of ATSNLAS (SEQ ID NO: 40); and (f) an HVR-L3 comprising the amino acid sequence of QQWTSNPPT (SEQ ID NO: 41). The methods described herein may also include administering an isolated anti-CD20 antibody that binds to the same epitope as an anti-CD20 antibody described above.

In certain aspects, the anti-CD20 antibody is rituximab (RITUXAN®). In other aspects, the anti-CD20 antibody is Y2B8 or Ibritumomab Tiuxetan (ZEVALIN®). In other aspects, the anti-CD20 antibody is tositumomab, (BEXXAR™). In other aspects, the anti-CD20 antibody is huMax-CD20 or ofatumumab (ARZERRA®). Examples of anti-CD20 antibodies useful for the methods of this invention and methods for making thereof are described in U.S. Pat. Nos. 5,736,137; 5,595,721; 5,677,180; in U.S. Pub. Nos: US 2003/0219433 and US 2003/0219433; and in PCT Pub. No: WO03/002607, expressly incorporated herein by reference. The anti-CD20 antibodies (e.g., rituximab) useful in this invention, including compositions containing such antibodies, may be used in combination with an anti-TIGIT antagonist antibody to treat a hematologic cancer (e.g., a lymphoma (e.g., a NHL, e.g., a relapsed or refractory DLBCL or a relapsed or refractory FL)).

An anti-CD20 antibody according to any of the above aspects may be a monoclonal antibody, comprising a chimeric, humanized, or human antibody. In one aspect, an anti-CD20 antibody is an antibody fragment, for example, a Fv, Fab, Fab′, scFv, diabody, or F(ab′)₂ fragment. In another aspect, the antibody is a full-length antibody, e.g., an intact IgG antibody (e.g., an intact IgG1 antibody) or other antibody class or isotype as defined herein.

In a further aspect, an anti-CD20 antibody according to any of the above aspects may incorporate any of the features, singly or in combination, as described in Sections 1-6 below.

1. Antibody Affinity

In certain aspects, an anti-TIGIT antagonist antibody, anti-CD20 antibody, and/or anti-CD38 antibody provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one aspect, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20©) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another aspect, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (kₒₙ) are calculated using a simple one-to-one Langmuir binding model (BIACORE© Evaluation Software version 3.2) by simultaneously fitting the association and dissociation sensorgrams. The equilibrium dissociation constant (K_{D}) is calculated as the ratio kₒₙ/kₒₙ. See, for example, Chen et al., J. Mol. Biol. 293:865-881 (1999). If the on-rate exceeds 10⁶M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain aspects, an anti-TIGIT antagonist antibody, anti-CD20 antibody, and/or anti-CD38 antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain aspects, an anti-TIGIT antagonist antibody, anti-CD20 antibody, and/or anti-CD38 antibody provided herein is a chimeric antibody. Certain chimeric antibodies are described, e.g., in U.S. Pat. No. 4,816,567; and Morrison et al. Proc. Nat. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain aspects, an anti-TIGIT antagonist antibody, anti-CD20 antibody, and/or anti-CD38 antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in L et al, Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Anti-TIGIT antagonist antibody, anti-CD20 antibodies, and/or anti-CD38 antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-TIGIT antagonist antibody, anti-CD20 antibodies, and/or anti-CD38 antibodies or antibody fragments isolated from human antibody libraries are considered human antibodies or human antibody fragments herein.

6. Antibody Variants

In certain aspects, amino acid sequence variants of the anti-TIGIT antagonist antibodies, anti-CD20 antibodies, and/or anti-CD38 antibodies of the invention are contemplated. As described in detail herein, anti-TIGIT antagonist antibodies, anti-CD20 antibodies, and/or anti-CD38 antibodies may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain aspects, anti-TIGIT antagonist antibody, anti-CD20 antibody, and/or anti-CD38 antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino aids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain aspects of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

II. Glycosylation Variants

In certain aspects, anti-TIGIT antagonist antibodies, anti-CD20 antibodies, and/or anti-CD38 antibodies of the invention can be altered to increase or decrease the extent to which the antibody is glycosylated. Addition or deletion of glycosylation sites to anti-TIGIT antagonist antibody, anti-CD20 antibody, and/or anti-CD38 antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention are made in order to create antibody variants with certain improved properties.

In one aspect, anti-TIGIT antagonist antibody, anti-CD20 antibody, and/or anti-CD38 antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some aspects, the methods of the invention involve administering to the subject in the context of a fractionated, dose-escalation dosing regimen an anti-TIGIT antagonist antibody (e.g., an anti-TIGIT antagonist antibody disclosed herein (e.g., tiragolumab)) and/or anti-CD20 antibody (e.g., rituximab) or anti-CD38 antibody (e.g., daratumumab) variant that comprises an aglycosylation site mutation. In some aspects, the aglycosylation site mutation reduces effector function of the antibody. In some aspects, the aglycosylation site mutation is a substitution mutation. In some aspects, the antibody comprises a substitution mutation in the Fc region that reduces effector function. In some aspects, the substitution mutation is at amino acid residue N297, L234, L235, and/or D265 (EU numbering). In some aspects, the substitution mutation is selected from the group consisting of N297G, N297A, L234A, L235A, D265A, and P329G. In some aspects, the substitution mutation is at amino acid residue N297. In a preferred aspect, the substitution mutation is N297A.

Anti-TIGIT antagonist antibody, anti-CD20 antibody, and/or anti-CD38 antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc Region Variants

In certain aspects, one or more amino acid modifications are introduced into the Fc region of an anti-TIGIT antagonist antibody (e.g., an anti-TIGIT antagonist antibody disclosed herein (e.g., tiragolumab)), anti-CD20 antibody (e.g., rituximab), and/or anti-CD38 antibody (e.g., daratumumab) of the invention, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain aspects, the invention contemplates an anti-TIGIT antagonist antibody, anti-CD20 antibody, or antibody anti-CD38 antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain aspects, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain aspects, the antibody comprises at least one further amino acid substitution. In one aspect, the further amino acid substitution is S228P, E233P, L234A, L235A, L235E, N297A, N297D, or P331S, and still in another aspect the at least one further amino acid substitution is L234A and L235A of the human IgG1 Fc region or S228P and L235E of the human IgG4 Fc region (see e.g., US 2012/0251531), and still in another aspect the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspect, an antibody variant comprises an Fc region with one or more amino acid substitutions which improve ADCC, e.g., substitutions at positions 298, 333, and/or 334 of the Fc region (EU numbering of residues).

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects the anti-TIGIT antagonist antibody (e.g., an anti-TIGIT antagonist antibody disclosed herein (e.g., tiragolumab)), anti-CD20 antibody (e.g., rituximab), and/or anti-CD38 antibody (e.g., daratumumab) comprises an Fc region comprising an N297G mutation.

In some aspects, the anti-TIGIT antagonist antibody (e.g., an anti-TIGIT antagonist antibody disclosed herein (e.g., tiragolumab)), anti-CD20 antibody (e.g., rituximab), and/or anti-CD38 antibody (e.g., daratumumab) comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some aspects, at least one of the one or more heavy chain constant domains is paired with another heavy chain constant domain. In some aspects, the CH3₁ and CH3₂ domains each comprise a protuberance or cavity, and wherein the protuberance or cavity in the CH3₁ domain is positionable in the cavity or protuberance, respectively, in the CH3₂ domain. In some aspects, the CH3₁ and CH3₂ domains meet at an interface between said protuberance and cavity. In some aspects, the CH2₁ and CH2₂ domains each comprise a protuberance or cavity, and wherein the protuberance or cavity in the CH2₁ domain is positionable in the cavity or protuberance, respectively, in the CH2₂ domain. In other aspects, the CH2₁ and CH2₂ domains meet at an interface between said protuberance and cavity. In some aspects, the anti-TIGIT antagonist antibody (e.g., an anti-TIGIT antagonist antibody disclosed herein (e.g., tiragolumab)), anti-CD20 antibody (e.g., rituximab), and/or anti-CD38 antibody (e.g., daratumumab) is an IgG1 antibody.

### IV. Cysteine Engineered Antibody Variants

In certain aspects, it is desirable to create cysteine engineered anti-TIGIT antagonist antibodies, anti-CD20 antibodies, and/or anti-CD38 antibodies, e.g., “thioMAbs,” in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain aspects, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

### V. Antibody Derivatives

In certain aspects, an anti-TIGIT antagonist antibody of the invention (e.g., an anti-TIGIT antagonist antibody or a variant thereof (e.g., tiragolumab)), anti-CD20 antibody of the invention (e.g., rituximab), and/or anti-CD38 antibody of the invention (e.g., daratumumab or a variant thereof) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another aspect, conjugates of an antibody and nonproteinaceous moiety that may be selectively heated by exposure to radiation are provided. In one aspect, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Production Methods

Anti-TIGIT antagonist antibodies (e.g., an anti-TIGIT antagonist antibody disclosed herein (e.g., tiragolumab)), anti-CD20 antibodies (e.g., rituximab), and/or anti-CD38 antibodies (e.g., daratumumab) of the invention may be produced using recombinant methods and compositions, for example, as described in U.S. Pat. No. 4,816,567, which is incorporated herein by reference in its entirety.

For recombinant production of an anti-TIGIT antagonist antibody, anti-CD20 antibody, and/or anti-CD38 antibody, nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Immunoconjugates

The invention also provides immunoconjugates comprising an anti-TIGIT antagonist antibody (e.g., an anti-TIGIT antagonist antibody disclosed herein (e.g., tiragolumab)), anti-CD20 antibody (e.g., rituximab), and/or anti-CD38 antibody (e.g., daratumumab) of the invention conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some aspects, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another aspect, an immunoconjugate comprises an anti-TIGIT antagonist antibody as described herein (e.g., tiragolumab), anti-CD20 antibody (e.g., rituximab), and/or anti-CD38 antibody (e.g., daratumumab) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an anti-TIGIT antagonist antibody as described herein (e.g., tiragolumab), an anti-CD20 antibody as described herein (e.g., rituximab), and/or an anti-CD38 antibody as described herein (e.g., daratumumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See W094/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A.).

### V. Pharmaceutical Compositions and Formulations

Any of the anti-TIGIT antagonist antibodies and anti-CD20 antibodies or anti-CD38 antibodies described herein can be used in pharmaceutical compositions and formulations. Pharmaceutical compositions and formulations of an anti-TIGIT antagonist antibody and an anti-CD20 antibody or anti-CD38 antibody can be prepared by mixing such antibodies having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules. The formulations to be used for in vivo administration are generally sterile. Sterility may be readily accomplished, e.g., by filtration through sterile filtration membranes.

### VI. Articles of Manufacture and Kits

In another aspect of the invention, an article of manufacture or a kit containing materials useful for the treatment, prevention, and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing, and/or diagnosing the condition and may have a sterile access port (for example the container may be an intravenous solution bag or a vial having a stopper pierceable by a hypodermic injection needle).

At least one active agent in the composition is an anti-TIGIT antagonist antibody of the invention. The label or package insert indicates that the composition is used for treating the condition of choice (e.g., cancer, e.g., a hematologic cancer, e.g., a myeloma (e.g., MM, e.g., a relapsed or refractory MM) or a lymphoma (e.g., a NHL, e.g., a relapsed or refractory DLBCL or a relapsed or refractory FL)). Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

In one aspect, provided is a kit including an anti-TIGIT antagonist antibody (e.g., an anti-TIGIT antagonist antibody disclosed herein (e.g., tiragolumab)), an anti-CD38 antibody (e.g., daratumumab), and a package insert comprising instructions to administer to the subject having a hematologic cancer (e.g., a myeloma (e.g., MM, e.g., a relapsed or refractory MM)) the anti-TIGIT antagonist antibody at a fixed dose of between about 30 mg to about 1200 mg and an anti-CD38 antibody at a dose of between about 8 mg/kg to about 24 mg/kg in a dosing regimen comprising at least nine dosing cycles, wherein (a) the anti-TIGIT antagonist antibody is administered once every three weeks and (b) the anti-CD38 antibody is administered once every week during each of dosing cycles 1-3, once every three weeks during each of dosing cycles 4-8, and once every four weeks beginning on dosing cycle 9.

In another aspect, provided is a kit including an anti-TIGIT antagonist antibody (e.g., an anti-TIGIT antagonist antibody disclosed herein (e.g., tiragolumab)), an anti-CD38 antibody (e.g., daratumumab), and a package insert comprising instructions to administer to the subject having a MM (e.g., a relapsed or refractory MM) the anti-TIGIT antagonist antibody at a fixed dose of 600 mg and an anti-CD38 antibody at a dose of 16 mg/kg in a dosing regimen comprising at least nine dosing cycles, wherein the length of each dosing cycle is 21 days, and wherein (a) the anti-TIGIT antagonist antibody is administered once every three weeks and (b) the anti-CD38 antibody is administered once every week during each of dosing cycles 1-3, once every three weeks during each of dosing cycles 4-8, and once every four weeks beginning on dosing cycle 9.

In another aspect, provided is a kit including tiragolumab, daratumumab, and a package insert comprising instructions to administer to the subject having a MM (e.g., a relapsed or refractory MM) tiragolumab at a fixed dose of 600 mg and daratumumab at a dose of 16 mg/kg in a dosing regimen comprising at least nine dosing cycles, wherein the length of each dosing cycle is 21 days, and wherein (a) tiragolumab is administered once every three weeks and (b) the daratumumab is administered once every week during each of dosing cycles 1-3, once every three weeks during each of dosing cycles 4-8, and once every four weeks beginning on dosing cycle 9.

In another aspect, provided is a kit including an anti-TIGIT antagonist antibody (e.g., an anti-TIGIT antagonist antibody disclosed herein (e.g., tiragolumab)), an anti-CD20 antibody (e.g., rituximab), and a package insert comprising instructions to administer to the subject having a hematologic cancer (e.g., a lymphoma (e.g., a NHL, e.g., a relapsed or refractory DLBCL or a relapsed or refractory FL)) an anti-TIGIT antagonist antibody at a fixed dose of between about 30 mg to about 1200 mg and an anti-CD20 antibody at a dose of between about 250 mg/m² to about 500 mg/m² in a dosing regimen comprising at least a first and a second dosing cycle, wherein (a) the anti-TIGIT antagonist antibody is administered once every three weeks; and (b) the anti-CD20 antibody is administered once every week.

In another aspect, provided is a kit including an anti-TIGIT antagonist antibody (e.g., an anti-TIGIT antagonist antibody disclosed herein (e.g., tiragolumab)), an anti-CD20 antibody (e.g., rituximab), and a package insert comprising instructions to administer to the subject having a lymphoma (e.g., a NHL, e.g., a relapsed or refractory DLBCL or a relapsed or refractory FL) an anti-TIGIT antagonist antibody at a fixed dose of 600 mg and an anti-CD20 antibody at a dose of 375 mg/m² in a dosing regimen comprising at least a first and a second dosing cycle, wherein the length of each dosing cycle is 21 days, and wherein (a) each dosing cycle comprises a single dose of the anti-TIGIT antagonist antibody is administered on or about day 1 of each dosing cycle, (b) the first dosing cycle comprises a first dose (C1D1), a second dose (C1D2), and a third dose (C1D3) of the anti-CD20 antibody, wherein the C1 D1, the C1 D2, and the C1 D3 are administered on or about days 1, 8, and 15, respectively, of the first dosing cycle, and (c) the second dosing cycle further comprises a single dose of the anti-CD20 antibody administered on or about day 1 of the second dosing cycle, and wherein the dosing regimen comprises a total of four doses of the anti-CD20 antibody.

In another aspect, provided is a kit including tiragolumab, rituximab, and a package insert comprising instructions to administer to the subject having a lymphoma (e.g., a NHL, e.g., a relapsed or refractory DLBCL or a relapsed or refractory FL) tiragolumab at a fixed dose of 600 mg and rituximab at a dose of 375 mg/m² in a dosing regimen comprising at least a first and a second dosing cycle, wherein the length of each dosing cycle is 21 days, and wherein (a) each dosing cycle comprises a single dose of tiragolumab administered on or about day 1 of each dosing cycle, (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle, and (c) the second dosing cycle further comprises a single dose of rituximab administered on or about day 1 of the second dosing cycle, and wherein the dosing regimen comprises a total of four doses of rituximab.

In another aspect, provided is a kit including an anti-TIGIT antagonist antibody (e.g., an anti-TIGIT antagonist antibody disclosed herein (e.g., tiragolumab)), an anti-CD20 antibody (e.g., rituximab), and a package insert comprising instructions to administer to the subject having a hematologic cancer (e.g., a lymphoma (e.g., a NHL, e.g., a relapsed or refractory DLBCL or a relapsed or refractory FL)) an anti-TIGIT antagonist antibody at a fixed dose of between about 30 mg to about 1200 mg and an anti-CD20 antibody at a dose of between about 250 mg/m² to about 500 mg/m² in a dosing regimen comprising at least a first, a second, and a third dosing cycle, wherein (a) the anti-TIGIT antagonist antibody is administered once every three weeks; and (b) the anti-CD20 antibody is administered once every week.

In another aspect, provided is a kit including an anti-TIGIT antagonist antibody (e.g., an anti-TIGIT antagonist antibody disclosed herein (e.g., tiragolumab)), an anti-CD20 antibody (e.g., rituximab), and a package insert comprising instructions to administer to the subject having a lymphoma (e.g., a NHL, e.g., a relapsed or refractory DLBCL or a relapsed or refractory FL) an anti-TIGIT antagonist antibody at a fixed dose of 600 mg and an anti-CD20 antibody at a dose of 375 mg/m² in a dosing regimen comprising at least a first, a second, and a third dosing cycle, wherein the length of each dosing cycle is 21 days, and wherein (a) each dosing cycle comprises a single dose of the anti-TIGIT antagonist antibody is administered on or about day 1 of each dosing cycle, (b) the first dosing cycle comprises a first dose (C1D1), a second dose (C1D2), and a third dose (C1D3) of the anti-CD20 antibody, wherein the C1 D1, the C1 D2, and the C1 D3 are administered on or about days 1, 8, and 15, respectively, of the first dosing cycle, (c) the second dosing cycle further comprises a first dose (C2D1), a second dose (C2D2), and a third dose (C2D3) of the anti-CD20 antibody administered on or about days 1, 8, and 15, respectively, of the second dosing cycle; (d) the third dosing cycle further comprises a first dose (C3D1) and a second dose (C3D2) of the anti-CD20 antibody administered on or about days 1 and 8, respectively, of the third dosing cycle, and wherein the dosing regimen comprises a total of eight doses of the anti-CD20 antibody.

In another aspect, provided is a kit including tiragolumab, rituximab, and a package insert comprising instructions to administer to the subject having a lymphoma (e.g., a NHL, e.g., a relapsed or refractory DLBCL or a relapsed or refractory FL) tiragolumab at a fixed dose of 600 mg and rituximab at a dose of 375 mg/m² in a dosing regimen comprising at least a first, a second, and a third dosing cycle, wherein the length of each dosing cycle is 21 days, and wherein (a) each dosing cycle comprises a single dose of tiragolumab administered on or about day 1 of each dosing cycle, (b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle, (c) the second dosing cycle further comprises a first dose (C2D1), a second dose (C2D2), and a third dose (C2D3) of rituximab administered on or about days 1, 8, and 15, respectively, of the second dosing cycle (d) the third dosing cycle further comprises a first dose (C3D1) and a second dose (C3D2) of rituximab administered on or about days 1 and 8, respectively, of the third dosing cycle, and wherein the dosing regimen comprises a total of eight doses of rituximab.

In another aspect, the invention features a kit including an anti-TIGIT antagonist antibody (e.g., an anti-TIGIT antagonist antibody disclosed herein (e.g., tiragolumab)) of the invention, an anti-CD38 antibody (e.g., daratumumab), and a package insert comprising instructions for using the anti-TIGIT antagonist antibody and anti-CD38 antibody for treating cancer (e.g., a hematologic cancer, e.g., a myeloma (e.g., MM, e.g., a relapsed or refractory MM)) in a subject according to any of the methods disclosed herein.

In another aspect, the invention features a kit including an anti-TIGIT antagonist antibody (e.g., an anti-TIGIT antagonist antibody disclosed herein (e.g., tiragolumab)) of the invention, anti-CD20 antibody (e.g., rituximab), and a package insert comprising instructions for using the anti-TIGIT antagonist antibody and anti-CD20 antibody for treating cancer (e.g., a hematologic cancer, e.g., a lymphoma (e.g., a NHL, e.g., a relapsed or refractory diffuse large B cell lymphoma or a relapsed or refractory follicular lymphoma)) in a subject according to any of the methods disclosed herein. In any of the above aspects, the subject may, for example, be a human. It is specifically contemplated that any of the anti-TIGIT antagonist antibodies and anti-CD20 antibodies or anti-CD38 antibodies described herein may be included in the kit.

In another aspect, provided is a kit including an anti-TIGIT antagonist antibody (e.g., an anti-TIGIT antagonist antibody disclosed herein (e.g., tiragolumab)) and a package insert comprising instructions to administer to the subject having a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL)) the anti-TIGIT antagonist antibody at a fixed dose of between about 30 mg to about 1200 mg in a dosing regimen comprising one or more dosing cycles, wherein the anti-TIGIT antagonist antibody is administered once every three weeks.

In another aspect, provided is a kit including an anti-TIGIT antagonist antibody (e.g., an anti-TIGIT antagonist antibody disclosed herein (e.g., tiragolumab)) and a package insert comprising instructions to administer to the subject having a MM (e.g., a relapsed or refractory MM) the anti-TIGIT antagonist antibody at a fixed dose of 600 mg in a dosing regimen comprising at one or more dosing cycles, wherein the length of each dosing cycle is 21 days, and wherein the anti-TIGIT antagonist antibody is administered once every three weeks.

In another aspect, provided is a kit including tiragolumab and a package insert comprising instructions to administer to the subject having a MM (e.g., a relapsed or refractory MM) tiragolumab at a fixed dose of 600 mg in a dosing regimen comprising one or more dosing cycles, wherein the length of each dosing cycle is 21 days, and wherein tiragolumab is administered once every three weeks. In some aspects, the instructions may further indicate that tiragolumab is to be administered as a monotherapy.

In another aspect, provided is a kit including an anti-TIGIT antagonist antibody (e.g., an anti-TIGIT antagonist antibody disclosed herein (e.g., tiragolumab)) and a package insert comprising instructions to administer to the subject having a NHL (e.g., a relapsed or refractory DLBCL or a relapsed or refractory FL)) the anti-TIGIT antagonist antibody at a fixed dose of 600 mg in a dosing regimen comprising at one or more dosing cycles, wherein the length of each dosing cycle is 21 days, and wherein the anti-TIGIT antagonist antibody is administered once every three weeks.

In another aspect, provided is a kit including tiragolumab and a package insert comprising instructions to administer to the subject having a NHL (e.g., a relapsed or refractory DLBCL or a relapsed or refractory FL)) tiragolumab at a fixed dose of 600 mg in a dosing regimen comprising one or more dosing cycles, wherein the length of each dosing cycle is 21 days, and wherein tiragolumab is administered once every three weeks. In some aspects, the instructions may further indicate that tiragolumab is to be administered as a monotherapy.

In another aspect, the invention features a kit including an anti-TIGIT antagonist antibody (e.g., an anti-TIGIT antagonist antibody disclosed herein (e.g., tiragolumab)) of the invention and a package insert comprising instructions for using the anti-TIGIT antagonist antibody for treating cancer (e.g., a hematologic cancer (e.g., a myeloma (e.g., a multiple myeloma (MM), e.g., a relapsed or refractory MM) or a lymphoma (e.g., a non-Hodgkin's lymphoma (NHL), e.g., a relapsed or refractory diffuse large B cell lymphoma (DLBCL) or a relapsed or refractory follicular lymphoma (FL))) in a subject according to any of the methods disclosed herein. In any of the above aspects, the subject may, for example, be a human. It is specifically contemplated that any of the anti-TIGIT antagonist antibodies described herein may be included in the kit.

### VII. Examples

The following are examples of the methods of the invention. It is understood that various other aspects may be practiced, given the general descriptions provided above.

### Example 1. Efficacy of an Anti-TIGIT Antagonist Antibody Alone or in Combination with an Anti-CD38 Antibody or an Anti-CD20 Antibody in Patients with Hematologic Cancers

To evaluate the efficacy and safety of treatment with an anti-TIGIT antagonist antibody (e.g., an anti-TIGIT antibody disclosed herein (e.g., tiragolumab)) in combination with an anti-CD38 antibody (e.g., daratumumab) or an anti-CD20 antibody (e.g., rituximab) in patients with a hematologic cancer (e.g., multiple myeloma (MM) (e.g., relapsed or refractory MM) or Non-Hodgkin's Lymphoma (NHL) (e.g., relapsed or refractory diffuse large B cell lymphoma (DLBCL) or follicular lymphoma (FL)), patients are enrolled in a phase Ia/Ib open-label, global, multicenter study.

Inclusion/Exclusion Criteria

To be eligible, patients must sign an informed consent form and be ≥18 years of age at the time of signing, have the ability to comply with the study's protocol, have an Eastern Cooperative Oncology Group (ECOG) performance status of 0 or 1, and a life expectancy of ≥12 weeks. Patients who have experienced adverse events from prior anti-cancer therapy, must have resolved to ≤Grade 1, excepting any grade alopecia, vitiligo, and anorexia, Grade ≤2 peripheral sensory or motor neuropathy, and endocrinopathy managed with replacement therapy.

Patients must also have AST and ALT ≤3× upper limit of normal (ULN), and total serum bilirubin of ≤1.5×ULN. Patients must also have alkaline phosphatase ≤2.5×ULN. Patients with documented liver or bone metastases may have alkaline phosphatase ≤5×ULN. Patients must also have a platelet count ≥75,000/μL without transfusion in the 14 days prior to first dose of study treatment, and an absolute neutrophil count (ANC) ≥1000/μL.

Patients must additionally consent to provide bone marrow biopsy and aspirate samples as necessary for evaluation and detailed in the protocol.

Women of childbearing potential must agree to remain abstinent from heterosexual intercourse or use contraceptive methods with a failure rate of <1% per year during the treatment period and for 5 months after the final dose of the anti-TIGIT antagonist antibody (e.g., tiragolumab), 3 months after the final dose of the anti-CD38 antibody (e.g., dratumumab), and 12 months after the final does of the anti-CD20 antibody (e.g., rituximab), whichever is later.

A woman is considered to be of childbearing potential if she is postmenarcheal, has not reached a postmenopausal state (≥12 continuous months of amenorrhea with no identified cause other than menopause), and is not permanently infertile due to surgery (i.e., removal of ovaries, fallopian tubes, and/or uterus) or another cause as determined by the investigator (e.g., Müllerian agenesis). The definition of childbearing potential may be adapted for alignment with local guidelines or regulations.

Examples of contraceptive methods with a failure rate of <1% per year include bilateral tubal ligation, male sterilization, hormonal contraceptives that inhibit ovulation, hormone-releasing intrauterine devices, and copper intrauterine devices. Hormonal contraceptive methods must be supplemented by a barrier method.

Men must agree to remain abstinent from heterosexual intercourse or use a condom, and refrain from donating sperm during the treatment period and for three months after the final dose of the anti-TIGIT antagonist antibody and/or the anti-CD38 antibody, and 5 months after the final dose of the anti-CD20 antibody, whichever is later, to avoid exposing an embryo.

The reliability of sexual abstinence should be evaluated in relation to the duration of the clinical trial and the preferred and usual lifestyle of the patient. Periodic abstinence (e.g., calendar, ovulation, symptothermal, or postovulation methods) and withdrawal are not acceptable methods of preventing drug exposure. If required per local guidelines or regulations, information about the reliability of abstinence will be described in the local Informed Consent Form.

Patients must not have used any chemotherapy, monoclonal antibody, radioimmunoconjugate, hormonal therapy, radiotherapy, and/or antibody-drug conjugate within 4 weeks prior to the first study drug administration.

Patients must not have had prior treatment with CAR-T therapy within 30 days before first study drug administration. Patients must not have had treatment with any chemotherapeutic agent, or treatment with any other anti-cancer agent (investigational or otherwise) within 6 weeks or 5 half-lives of the drug, whichever is shorter, prior to first study drug administration, with the following exceptions:

(a) prior treatment with cytokine therapy and/or cancer vaccines within 6 weeks or five half-lives of the drug, whichever is shorter before the first study drug administration;

(b) prior treatment with immune checkpoint inhibitor, including, including but not limited to anti-CTLA4, anti-PD-1, and/or anti-PD-L1 therapeutic antibodies, within 4 weeks or five half-lives of the drug, whichever is shorter, before first study drug administration;

(c) prior cancer immunotherapy not explicitly described in this protocol should be discussed with the Medical Monitor to determine potential eligibility;

(d) hormone-replacement therapy or oral contraceptives;

(e) herbal therapy within 7 days before first study drug administration; and/or

(f) palliative radiotherapy for painful metastases or metastases in potentially sensitive locations (e.g., epidural space) within 14 days prior to first study drug administration.

Patients with any history of an immune related Grade 4 adverse event attributed to prior cancer immunotherapy (other than endocrinopathy managed with replacement therapy or asymptomatic elevation of serum amylase or lipase) are ineligible. Additionally, patients are ineligible if they have had Grade 3 adverse events, with the exception of Grade 3 endocrinopathy managed with replacement therapy or asymptomatic elevation of serum amylase or lipase, that resulted in permanent discontinuation of the prior immunotherapeutic agent and/or occurred ≤6 months prior to Cycle 1, Day 1 and/or Grade 1-2 adverse events that did not resolve to baseline after treatment discontinuation associated with prior immunotherapeutic agents, excepting grade 2 peripheral sensory or motor neuropathy, any grade alopecia or vitiligo, and/or endocrinopathy managed with replacement therapy. Patients treated with corticosteroids for immune-related adverse events must demonstrate absence of related symptoms or signs for 4 weeks following discontinuation of corticosteroids.

Patients who have had prior treatment with any anti-TIGIT agent are also ineligible. Patients must not have had prior allogenic SCT, autologous SCT within 100 days prior to the first study drug administration, or prior solid organ transplantation.

Patients with active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis are ineligible.

Patients with a history of autoimmune-related hypothyroidism who are on thyroid-replacement hormone, those with controlled Type 1 diabetes mellitus who are on a stable insulin regimen, or with a history of disease-related immune thrombocytopenic purpura or autoimmune hemolytic anemia are eligible.

Patients with eczema, psoriasis, lichen simplex chronicus, or vitiligo with dermatologic manifestations only (e.g., patients with psoriatic arthritis are excluded) are eligible for the study provided all of following conditions are met:

(a) rash must cover <10% of body surface area

(b) disease is well controlled at baseline and requires only low-potency topical corticosteroids

(c) no occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.

Patients must not have a history of idiopathic pulmonary fibrosis, organizing pneumonia (e.g., bronchiolitis obliterans), drug-induced pneumonitis, or idiopathic pneumonitis, or evidence of active pneumonitis on screening chest CT scan (excepting a history of radiation pneumonitis in the radiation field (fibrosis)), confirmed progressive multifocal leukoencephalopathy (PML), leptomeningeal disease, severe allergic or anaphylactic reactions to monoclonal antibody therapy (or recombinant antibody-related fusion proteins) or known hypersensitivity to CHO-cell products, or of other malignancy that could affect compliance with the protocol or interpretation of results. Patients must also not have spinal cord compression not definitively treated with surgery and/or radiation or previously diagnosed and treated spinal cord compression without evidence that disease has been clinically stable for >2 weeks prior to screening.

Malignancies other than disease under study within 5 years prior to first study drug administration, with the exception of those with a negligible risk of metastasis or death (such as adequately treated carcinoma in situ of the cervix, basal or squamous cell skin cancer, localized prostate cancer, or ductal carcinoma in situ) are disqualifying. Patients with a history of curatively treated basal or squamous cell carcinoma of the skin or in situ carcinoma of the cervix, or those with a malignancy that has been treated with curative intent will also be allowed if the malignancy has been in remission without treatment for ≥2 years prior to first anti-TIGIT antagonist antibody administration.

Significant cardiovascular disease (e.g., New York Heart Association Class II or higher cardiac disease, myocardial infarction within the last 3 months, unstable arrhythmias, or unstable angina), significant active pulmonary disease (e.g., bronchospasm and/or obstructive pulmonary disease), and uncontrolled pleural effusion, pericardial effusion, or ascites requiring recurrent drainage procedures (once monthly or more frequently) is disqualifying. Patients with indwelling catheters (e.g., PleurX catheters) are allowed. Major surgery within 4 weeks prior to first study drug administration (excluding tumor biopsies, bone marrow biopsies, and superficial lymph node biopsies for diagnosis), and/or known active bacterial, viral, fungal, mycobacterial, parasitic, or other infection (excluding fungal infections of nail beds) at study enrollment, or any major episode of infection requiring treatment with IV antibiotics or hospitalization (relating to the completion of the course of antibiotics) within 4 weeks prior to first anti-TIGIT antagonist antibody administration are disqualifying. Additionally excluded are patients having recent infections not meeting the above criteria for severe infections, including the following:

(a) signs or symptoms of infection within 2 weeks prior to first study drug administration; and/or

(b) received oral or IV antibiotics within 2 weeks prior to first study drug administration. Patients receiving prophylactic antibiotics (e.g., for prevention of a urinary tract infection or chronic obstructive pulmonary disease) are eligible.

Patients having uncontrolled tumor-related pain including symptomatic lesions amenable to palliative radiotherapy (e.g., bone metastases or metastases causing nerve impingement) should be treated prior to enrollment. Patients having asymptomatic metastatic lesions whose further growth would likely cause functional deficits or intractable pain (e.g., epidural metastasis that is not currently associated with spinal cord compression) should be considered for loco-regional therapy if appropriate prior to enrollment.

Patients may not have an active Epstein-Barr virus (EBV) infection or a known or suspected chronic active EBV infection. If a patient has positive serology for EBV IgG and/or is positive for Epstein-Barr nuclear antigen (EBNA), then EBV IgM testing and/or EBV PCR is required for consideration of eligibility. If the patient has positive serology for EBV IgG and/or is positive for EBNA, they must be negative for EBV IgM and/or negative by EBV PCR.

If a patient has a negative HBsAg test and a positive total HBcAb test at screening, an HBV DNA test must also be performed to determine if the patient has an HBV infection. Additional EBV serology tests are performed for patients who subsequently experience an acute inflammatory event, e.g., systemic inflammatory response syndrome, while receiving study treatment.

Patients who are positive for HCV antibody must be negative for HCV by PCR to be eligible for study participation.

Patients must not have active tuberculosis.

Patients may not have a known history of HIV seropositivity.

Patients may not be treated with a live, attenuated vaccine within 4 weeks prior to initiation of study treatment, or anticipation of need for such a vaccine during study treatment or within 5 months after the final dose of study treatment. Influenza vaccination should be given during influenza season only. Patients must not receive live, attenuated influenza vaccine (e.g., FluMist) within 4 weeks prior to first study drug administration or at any time during the study, and for 5 months after the last study treatment.

Patients may not be treated with systemic corticosteroids or other systemic immunosuppressive medications (including but not limited to prednisone >10 mg/day, cyclophosphamide, azathioprine, methotrexate, thalidomide, and anti-tumor necrosis factor agents) within 2 weeks prior to first dose of study treatment. Acute, low-dose, systemic immunosuppressant medications (e.g., single dose of dexamethasone for nausea or B symptoms) may be enrolled. The use of inhaled corticosteroids, mineralocorticoids for management of orthostatic hypotension, and/or physiologic doses of corticosteroids for management of adrenal insufficiency is permitted.

Those who have a history of illicit drug or alcohol abuse in the past year prior to screening, or those that have a serious medical condition or abnormality in clinical laboratory tests that may preclude the patient's safe participation in and completion of the study, or which could affect compliance with the protocol or interpretation of results are also excluded.

Those who are pregnant or breastfeeding, or intending to become pregnant during the study or within 5 months after the final dose of the anti-TIGIT antagonist antibody, 3 months after the final dose of the anti-CD38 antibody, and 12 months after the final dose of the anti-CD20 antibody are not eligible. Women of childbearing potential must have a negative serum pregnancy test result within 14 days prior to initiation of study drug.

Patients having any serious medical condition, metabolic dysfunction, physical examination finding, and/or abnormality in clinical laboratory tests that, in the investigator's or Medical Monitor's judgment, precludes the patient's safe participation in and completion of the study, or which could affect compliance with the protocol or interpretation of results, or which may render the patient at high risk from treatment complications are also excluded.

Concomitant Therapy

Certain concomitant therapies are permitted. Concomitant therapies include any medication (e.g., prescription drugs, over the counter drugs, vaccines, herbal or homeopathic remedies, nutritional supplements) used by a patient in addition to protocol-mandated study treatment from seven days prior to initiation of study treatment to the treatment discontinuation visit. Patients are permitted to use the following concomitant therapies during the study.

The use of oral contraceptives and hormone-replacement therapy is acceptable while the patient is enrolled in the study. Concomitant use of hematopoietic growth factors such as erythropoietin, G-CSF (e.g., filgrastim or pegfilgrastim), granulocyte/macrophage colony-stimulating factor (e.g., sargramostim), or thrombopoietin (e.g., oprelvekin or eltrombopag) is also permitted. Initiation or dose and schedule modifications of hematopoietic growth factors is allowed.

Systemic corticosteroids and other immune-modulating medications may, in theory, attenuate the potential beneficial immunologic effects of treatment with the anti-TIGIT antagonist antibody but should be administered at the discretion of the treating physician in line with the management guidelines. Premedication for the anti-TIGIT antagonist antibody may be administered for Cycles ≥2 at the discretion of the treating physician after consultation with the medical monitor. For patients who receive the anti-CD20 antibody, in addition to premedication with antihistamines and antipyretics, an additional glucocorticoid (e.g., 100 mg IV prednisone or prednisolone or equivalent) is allowed at the investigator's discretion. The use of inhaled corticosteroids and mineralocorticoids (e.g., fludrocortisone) for patients with orthostatic hypotension or adrenocortical insufficiency is also allowed. Physiologic doses of corticosteroids for adrenal insufficiency are allowed.

Megestrol administered as an appetite stimulant is acceptable while the patient is enrolled in the study. Anti-infective prophylaxis for viral, fungal, bacterial or pneumocystis infections is also permitted. Cannabinoids are permitted only if obtained in accordance with local regulations.

Premedication with antihistamines, antipyretics, and/or analgesics may be administered only for the second and subsequent infusion of the anti-TIGIT antagonist antibody at the discretion of the investigator. Premedication with an antihistamine and acetaminophen is required for all patients receiving the anti-TIGIT antagonist antibody in combination with the anti-CD20 antibody or the anti-CD38 antibody.

In general, investigators should manage a patient's care (including preexisting conditions) with supportive therapies other than those defined as cautionary or prohibited therapies as clinically indicated. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists.

Any investigational therapy (excepting protocol-mandated study treatment) is prohibited within 28 days prior to initiation of the study treatment and during study treatment. Concomitant therapy intended for the treatment of cancer (including, but not limited to, chemotherapy, hormonal therapy, immunotherapy, radiotherapy, and herbal therapy) is prohibited for various time periods prior to starting study treatment, depending on the agent, and during study treatment, until disease progression is documented and the patient has discontinued study treatment. Certain forms of radiotherapy may be considered for pain palliation if patients are deriving benefit (e.g., treatment of known bony metastases) and provided they do not compromise assessments of tumor target lesions. Study drug administration may be continued during radiotherapy. Patients experiencing a mixed response requiring local therapy (e.g., surgery, stereotactic radiosurgery, radiotherapy, radiofrequency ablation) for control of lesions may still be eligible to continue study treatment, at the discretion of the investigator and after discussion with the Medical Monitor. Subsequent tumor assessments may need to take the local treatment into account in determining overall response.

Biologic agents other than hematopoietic growth factors, any therapies intended for the treatment of lymphoma, leukemia, or multiple myeloma, and immunosuppressive medications (e.g., cyclophosphamide, azathioprine, methotrexate, and thalidomide), immunostimulatory agents (e.g., IFN-α, IFN-γ, or interleukin-2) are also prohibited.

In addition, all patients (including those who discontinue the study early) should not receive other immunostimulatory agents for 5 half-lives (12 weeks) after the last dose of the anti-TIGIT antagonist antibody. Live, attenuated vaccines (e.g., FLUMIST®) are prohibited within 4 weeks prior to initiation of study treatment, during study treatment, and for 5 months after the final dose of study treatment. Receptor activator of nuclear factor kappa B (RANK) inhibitor (i.e., denosumab) is also prohibited. Patients who are receiving denosumab prior to enrollment must be willing and eligible to receive a bisphosphonate instead while on study.

Safety Endpoints

The phase Ia portion will evaluate the safety, pharmacokinetics, pharmacodynamics, and preliminary anti-tumor activity of the anti-TIGIT antagonist antibody when administered as a single agent in patients with recurrent or relapsed MM, DLBCL, or FL. The phase Ib portion will evaluate the safety, pharmacokinetics, pharmacodynamics, and preliminary anti-tumor activity of the anti-TIGIT antagonist antibody in combination with an anti-CD38 antibody in patients with recurrent or relapsed MM, or in combination with an anti-CD20 antibody in patients with relapsed or recurrent DLBCL or FL.

Safety will be evaluated by recording the incidence and severity of adverse events, with severity determined according to the National Cancer Institute Common Terminology Criteria for Adverse Events (NCI CTCAE), version 5.0 and by the change in baseline in targeted vital signs, clinical laboratory test results, and physical examination findings. The percentage of patients having adverse events may be assessed during screening, during each of cycles 1-21, and upon discontinuation of study treatment.

Pharmacokinetic Analyses

The pharmacokinetic (PK) profiles of the anti-TIGIT antagonist antibody when administered as a single agent, or in combination with an anti-CD38 or an anti-CD20 antibody will be determined by examining the serum concentration (e.g., Cₘₐₓ and Cₘᵢₙ) of the anti-TIGIT antagonist antibody at various timepoints (e.g., at each of cycles 1, 2, 3, 4, 8, 16, 17, and every 8 cycles thereafter (e.g., pre-infusion, at 30 (±10) minutes, 3 hours (±15) minutes and/or 24 (±6) hours following infusion), and at treatment discontinuation). The serum concentration of the anti-CD38 and anti-CD20 antibodies when administered in combination with the anti-TIGIT antagonist antibody will also be measured. Data may be compared with historical data, as these results will provide preliminary information on whether the anti-TIGIT antagonist antibody, the anti-CD38 antibody, and the anti-CD20 antibody PK are altered by co-administration of the other agent.

Activity Analyses

The following analyses to determine the activity of anti-TIGIT antagonist antibody as a single agent or in combination with the anti-CD38 antibody or anti-CD20 antibody will be based on the definitions of objective response according to the International Myeloma Working Group Uniform Response (IMWG) criteria (adapted from Durie et al. 2015 and Kumar et al. 2016) for MM or the Lugano Response Criteria for Malignant Lymphoma for DLBCL/FL. Response assessments will be assessed on the basis of physical examinations. CT scans, fluorodeoxyglucose (FDG) positron emission tomography (PET) scans, PET/CT scans, and/or MRI scans, and bone marrow examinations, according to the IMWG response criteria for MM and the Lugano classification for DLBCL/FL.

Response assessment data, duration of objective response (DOR), progression-free survival (PFS), and overall survival (OS) will be recorded for all patients with measurable disease by dose level or tumor type.

The analysis of objective response rate (ORR) will include patients in the Phase Ia or Phase Ib study who received any amount of the study treatment and have measurable disease at baseline. Objective response is defined as a complete response (CR) or partial response (PR), as determined by investigator assessment and confirmed by repeat assessment 4 weeks after initial documentation. Patients with missing baseline or no response assessments will be classified as non-responders. Objective response rate will be estimated and summarized by tumor type and by dose, if applicable.

Among patients with an objective response, duration of objective response will be defined as the time from the initial complete or partial response to the time of disease progression or death, whichever occurs first. For patients who do not die or experience disease progression before the end of the study or who are lost to follow-up, duration of objective response will be censored at the day of the last tumor assessment.

The analyses of PFS will include patients who have received any amount of study treatment. PFS is defined as the time from enrollment or the first day of study treatment with the anti-TIGIT antagonist antibody (Cycle 1, Day 1 in Phase Ia or Phase Ib) until documented disease progression or death, whichever occurs first. For patients who do not have documented progressive disease or death before the end of the study or who are lost to follow-up, PFS will be censored at the day of the last tumor assessment.

Immunogenicity Analyses

The immunogenicity analysis population will consist of all patients with at least one anti-TIGIT antagonist antibody anti-drug antibody (ADA) assessment. Patients will be grouped according to treatment received or, if no treatment is received prior to study discontinuation, according to treatment assigned.

The numbers and proportions of anti-TIGIT antagonist antibody ADA-positive patients and anti-TIGIT antagonist antibody ADA-negative patients at baseline (baseline prevalence) and after drug administration (post baseline incidence) will be summarized by treatment group. The presence of ADAs to the anti-TIGIT antagonist antibody and percentage of patients with ADAs to the anti-TIGIT antagonist antibody may be assessed at each of cycles 1, 2, 4, 8, 16, 17, and every 8 cycles thereafter, and at the discontinuation of study treatment. When determining post baseline incidence, patients are considered to be ADA positive if they are ADA negative or have missing data at baseline but develop an ADA response following study drug exposure (treatment-induced ADA response), or if they are ADA positive at baseline and the titer of one or more post baseline samples is at least 0.60 titer unit greater than the titer of the baseline sample (treatment-enhanced ADA response). Patients are considered to be ADA negative if they are ADA negative or have missing data at baseline and all post baseline samples are negative, or if they are ADA positive at baseline but do not have any post baseline samples with a titer that is at least 0.60 titer unit greater than the titer of the baseline sample (treatment unaffected).

Patients in the Phase Ib portion of the study who are treated with the anti-CD38 antibody or anti-CD20 antibody may be assessed for ADAs against the anti-CD38 antibody or the anti-CD20 antibody respectively. The relationship between ADA status and safety, activity, PK, and biomarker endpoints may be analyzed and reported via descriptive statistics as appropriate.

Biomarkers

Patient samples, including archival tumor tissues and bone marrow biopsies, as well as serum, plasma, and whole blood are collected for exploratory biomarker assessments for all patients in the study.

The objective of the exploratory biomarker assessments is to identify and/or evaluate biomarkers that may be predictive of response to the anti-TIGIT antagonist antibody as a single agent or in combination with an anti-CD38 antibody or anti-CD20 antibody (i.e., predictive biomarkers); are early surrogates of activity; are associated with progression to a more severe disease state (i.e., prognostic biomarkers); are associated with acquired resistance to the anti-TIGIT antagonist antibody as a single agent and in combination with an anti-CD38 antibody or anti-CD20 antibody; are associated with susceptibility to developing adverse events or can lead to improved adverse event monitoring or investigation (i.e., safety biomarkers); can provide evidence of activity of the anti-TIGIT antagonist antibody as a single agent and in combination with an anti-CD38 antibody or anti-CD20 antibody (i.e., pharmacodynamic biomarkers); or can increase the knowledge and understanding of disease biology and drug safety, and will be evaluated on the basis of the relationship between biomarkers in blood, bone marrow, and tumor tissue and safety, PK, activity, immunogenicity, or other endpoints.

Blood samples will be collected for DNA extraction to enable whole genome sequencing (WGS) or whole exome sequencing (WES) to identify variants that are predictive of response to study drug, are associated with progression to a more severe disease state, are associated with susceptibility to developing adverse events, can lead to improved adverse event monitoring or investigation, or can increase the knowledge and understanding of disease biology and drug safety.

### Example 2. Efficacy of an Anti-TIGIT Antagonist Antibody Alone or in Combination with an Anti-CD38 Antibody in Patients with Multiple Myeloma

To evaluate the efficacy and safety of treatment with an anti-TIGIT antagonist antibody (e.g., an anti-TIGIT antibody disclosed herein (e.g., tiragolumab)) in combination with an anti-CD38 antibody (e.g., daratumumab) in patients with a multiple myeloma (MM) (e.g., relapsed or refractory MM), patients are enrolled in a phase Ia/Ib open-label, global, multicenter study.

Inclusion/Exclusion Criteria

In addition to the inclusion and exclusion criteria disclosed in Example 1, above, patients must also meet the following eligibility requirements.

For patients with MM who have a >50% myeloma involvement in the bone marrow, a platelet count of ≥50,000 μL prior to enrollment is allowed. Subjects may not have received a platelet transfusion within 72 hours of the platelet count used for eligibility.

Patients may use growth factor support to achieve ANC eligibility criteria as described in Example 1, above. Patients may not have received a growth factor within the previous seven days prior to the ANC used for eligibility.

Patients must have total hemoglobin 8 g/dL; patients may receive red blood cell (RBC) transfusions or erythropoietic agents to meet this criteria. Patients who do not meet criteria for hematologic function due to extensive marrow involvement of MM and/or disease-related cytopenias (e.g., immune thrombocytopenia) may be enrolled into the study after discussion with and after approval of the Medical Monitor. Patients must have serum creatinine ≤2.0 mg/dL and creatinine clearance ≥30 mL/min (calculated or per 24-hr urine collection).

Serum calcium (corrected for albumin) level at or below the ULN (treatment of hypercalcemia is allowed and patients are eligible to enroll if the calcium level returns to normal with standard treatment).

To be eligible for treatment with an anti-TIGIT antagonist antibody monotherapy, patients must have R/R MM for which no established therapy for MM is appropriate and available or be intolerant to those established therapies. To be eligible for treatment with an anti-TIGIT antagonist antibody in combination with an anti-CD38 antibody, patients must have received at least 3 prior lines of therapy (e.g., including a proteasome inhibitor, an immunomodulatory drug (IMiD), and an anti-CD38 antibody). Patients must also undergo blood type, Rh, and indirect anti-globulin test (IAT; Indirect Coombs Test) assays before the first dose of the anti-CD38 antibody (e.g., daratumumab).

A line of therapy consists of ≥1 complete cycle of a single agent, a regimen consisting of a combination of several drugs, or a planned sequential therapy of various drugs (e.g., induction therapy followed by stem-cell transplantation (SCT) is considered 1 line of therapy. Documented evidence of progressive disease (as defined by the IMWG criteria) on or after the last prior therapy, or patients who were intolerant to the last prior therapy are eligible. Patients who are intolerant of daratumumab are not eligible.

Measurable disease is defined as at least one of the following:

(a) serum monoclonal protein (M-protein) ≥0.5 g/dL ≥5 g/L)

(b) urine M-protein ≥200 mg/24 hr

(c) serum free light chain (SFLC) assay: Involved SFLCs ≥10 mg/dL (≥100 mg/L) and an abnormal SFLC ratio (<0.26 or >1.65)

Patients must not have primary or secondary plasma cell leukemia as defined by an absolute plasma cell count exceeding 2000/μL or 20% of the peripheral blood white cells. Additionally, patients must not have current or history of CNS involvement by MM or have allergies or hypersensitivities to any components of the anti-CD38 antibody (e.g., daratumumab) formulation.

Study Treatment Dosage and Administration

Anti-TIGIT Antagonist Antibody as a Single Agent

During treatment, patients receive a fixed dose of 600 mg of an anti-TIGIT antagonist antibody (e.g., an anti-TIGIT antibody disclosed herein, e.g., tiragolumab) administered by intravenous infusion every 3 weeks (q3w) (21±3 days). The anti-TIGIT antagonist antibody (e.g., an anti-TIGIT antibody disclosed herein, e.g., tiragolumab) is administered on Day 1 of each 21-day dosing cycle. Prior to the first infusion of the anti-TIGIT antibody, the patient's vital signs (e.g., pulse rate, respiratory rate, blood pressure, and temperature) are recorded within 60 minutes before starting the infusion. The first infusion of the anti-TIGIT antibody (e.g., an anti-TIGIT antibody disclosed herein) is administered over 60 (±10) minutes. During this time, the patient's vital signs (pulse rate, respiratory rate, blood pressure, and temperature) are recorded at 15-minute intervals. Following infusion, the patient is observed for 60 minutes, during which time, the vital signs are monitored at 30 (±10) minutes after the infusion of the anti-TIGIT antagonist antibody).

If no infusion-associated adverse events are experienced during the first infusions of the anti-TIGIT antagonist antibody (e.g., an anti-TIGIT antibody disclosed herein, e.g., tiragolumab), subsequent infusions can be administered over 30 (±10) minutes. Additionally, the post-infusion observation periods may be reduced to 30 (±10) minutes. Pre-infusion recordation of vital signs shall continue to be recorded within 60 minutes prior to the start of infusion of the anti-TIGIT antagonist antibody (e.g., an anti-TIGIT antibody disclosed herein, e.g., tiragolumab).

If the patient experiences an infusion-related reaction (IRR) during any infusion of the anti-TIGIT antagonist antibody (e.g., an anti-TIGIT antibody disclosed herein, e.g., tiragolumab), premedication with an antihistamine and/or antipyretic may be administered for subsequent cycles, and the patient's vital signs are to be recorded at 15 (±10) minutes after the infusion.

Anti-TIGIT Antagonist Antibody in Combination with an Anti-CD38 Antibody

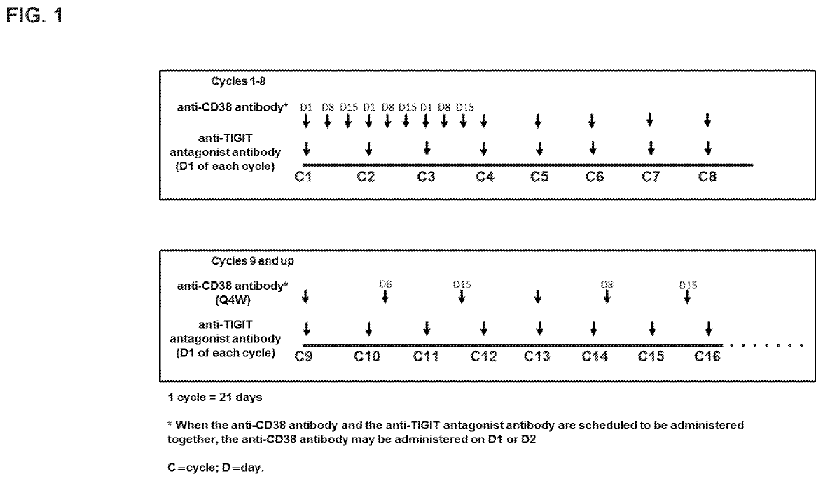

During treatment, patients receive a fixed dose of 600 mg of an anti-TIGIT antagonist antibody (e.g., an anti-TIGIT antibody disclosed herein, e.g., tiragolumab) administered by intravenous infusion every 3 weeks (q3w) (21±3 days). The anti-CD38 antibody (e.g., daratumumab) is administered by intravenous infusion at a dose of 16 mg/kg actual body weight weekly for a total of nine does, then every three weeks for a total of five doses, then every four weeks from week 25 onward until disease progression. The anti-CD38 antibody may be administered on day 1 or day 2 of each three-week cycle when the anti-TIGIT antagonist antibody and the anti-CD38 antagonist antibody are scheduled to be administered on the same day; however, if the anti-CD38 antibody and the anti-TIGIT antagonist antibody are administered on the same day, the anti-TIGIT antagonist antibody should be administered first (FIG. 1).

Antiviral prophylaxis to prevent herpes zoster reactivation should commence within one week after the first infusion of the anti-CD38 antibody, and continue for 3 months following treatment. Short and long-acting bronchodilators and inhaled corticosteroids may be administered as post-infusion medications for patients with a history of chronic obstructive pulmonary disease. These agents may be discontinued after the first four infusions in the absence of any major IRRs.

On the days of administration, the anti-TIGIT antagonist antibody is administered as described above. A pre-infusion regimen of 100 mg IV methylprednisolone (or equivalent), 650-1000 mg oral acetaminophen, and 25-50 mg oral or IV diphenhydramine (or equivalent) is administered to the patient one to three hours prior to the administration of the anti-CD38 antibody. Prior to the infusion of the anti-CD-38 antibody, the patient's vital signs (e.g., pulse rate, respiratory rate, blood pressure, and temperature) are recorded within 60 minutes before starting the infusion. The anti-CD38 antibody is diluted to a volume of 1000 mL and infused at a rate of 50 mL/hour for the first hour. In the absence of infusion reactions, the infusion rate may be increased by 50 mL/hour every hour, to a maximum rate of 200 mL/hour. The patient's vital signs are recorded every 15 (±5) minutes during the first two hours of every infusion, and then every 60 minutes thereafter for the remainder of the infusion. The patient's vital signs are recorded once again at the end of the infusion. Following infusion, the patient is observed for 60 minutes, during which time the vital signs are monitored as described above. Oral corticosteroid (20 mg methylprednisolone or equivalent dose of an intermediate-acting or long-acting corticosteroid) is administered on each of the two days following the administration of the anti-CD38 antibody, beginning the day after the infusion.

For subsequent infusions, pre-infusion medications are administered and vital signs are recorded as described above. If no IRRs are experienced during the first three hours of the first infusion, a dilution volume of 500 mL may be used, otherwise, a dilution volume of 1000 mL should be used. For the second infusion, anti-CD38 antibody infusion rates are the same as those used for the first infusion. For the third infusion onward, if there were no IRRs during a final infusion rate of 100 mL/hr in the first two infusions, a modified infusion orate of 100 mL/hour for the first hour with an increase of 50 mL/hour every hour to a maximum rate of 200 mL/hour may be used. Vital signs are recorded during the infusion as described above. Oral corticosteroids are administered post-infusion as described above. In the absence of IRRs, the post-infusion observation is conducted as described above. If the patient experienced IRRs in the previous infusion, the patient's vital signs are recorded at 15 (±10) minutes after the infusion.

Activity Analyses

The activity of the anti-TIGIT antagonist antibody as a single agent or in combination with the anti-CD38 antibody will be evaluated on the basis of ORR, DOR, PFS, and OS according to the International Myeloma Working Group Uniform Response (IMWG) criteria, as disclosed in Durie et al. Leukemia. 20(9):1467-73 (2006, Durie et al. Leukemia. 29:2416-7 (2015) and Kumar et al. Lancet Oncol. 17:e328-46 (2016), and as described in Tables 2 and 3. For MM, ORR is defined as the proportion of patients with a best overall response of stringent complete response (sCR), complete response (CR), very good partial response (VGPR), or partial response (PR), as defined by the IMWG criteria. ORR may be assessed during screening, during each of cycles 1-21, and upon discontinuation of study treatment. DOR is defined as the time from the first observation that a patient achieved a response (sCR, CR, VGPR, or PR), until the date of first recorded progression or death from any cause during the study (defined as within 30 days after the last dose of study drug), whichever occurs first. PFS is defined as the time from enrollment to the first occurrence of disease progression (per IMWG criteria) or death from any cause during the study (defined as within 30 days after the last dose of study drug), whichever occurs first.

TABLE 2
Response Categories According to IMWG Uniform Response Criteria

| | |
|---|---|
| Response | |
| Subcategory | Response Criteria |

All response categories require two consecutive assessments made any time
before starting any new therapy.

| | |
|---|---|
| sCR | CR as defined below, plus: |
| | Normal FLC ratio and absence of clonal cells in BM by immunohistochemistry |
| | (kappa/lamda ratio ≤ 4:1 or ≥ 1:2 for kappa and lambda patients, respectively after |
| | counting ≥100 plasma cells) ^{a} |
| CR | No evidence of initial monoclonal protein isotype(s) on immunofixation of the serum |
| | and urine, ^{b} disappearance of any soft tissue plasmacytomas, and ≤5% plasma cells |
| | in BM |
| VGPR | Serum and urine M-protein detectable by immunofixation but not on electrophoresis; |
| | or ≥90% reduction in serum M-protein- plus urine M-protein level < 100 mg/24 hr |
| PR | ≥50% reduction of serum M-protein and reduction in 24 hr urinary M-protein- by |
| | ≥90% or to <200 mg/24 hr. |
| | If the serum and urine M-protein are unmeasurable, a ≥50% decrease in the |
| | difference between involved and uninvolved FLC levels is required in place of |
| | the M-protein criteria. |
| | If serum and urine M-protein are unmeasurable and serum FLC assay is also |
| | unmeasurable, ≥50% reduction in plasma cells is required in place of M- |
| | protein, provided baseline BM plasma cell percentage was ≥30%. |
| | In addition to the above listed criteria, if present at baseline, a ≥50% reduction |
| | in the size (SPD) ^{c} of soft tissue plasmacytomas is also required. |
| MR | ≥25% but ≤49% reductions of serum M protein and reduction in 24-hour urine M- |
| | protein by 50%-89% |
| | In addition to the above criteria, if present at baseline, 25%-49% reduction in |
| | the size (SPD) ^{c} of soft tissue plasmacytomas is also required. |
| SD | Not meeting criteria for MR, CR, VGPR, PR, or PD |

TABLE 3
Disease Progression and Relapse According to IMWG Uniform Response Criteria

| Relapse | |
|---|---|
| Subcategory | Relapse Criteria |
| PD ^{d,e} | Any increase of ≥25% from lowest response value in any one of the following: |
| | Serum M-protein (absolute increase must be ≥0.5 g/dL) |
| | Serum M-protein increase ≥1 g/dL, if the lowest M component was ≥5 g/dL |
| | Urine M-protein (absolute increase must be ≥200 mg/24 hr) |
| | In patients without measurable serum and urine M-protein levels: the difference |
| | between involved and uninvolved FLC levels (absolute increase must be >10 |
| | mg/dL) |
| | In patients without measurable serum and urine M-protein levels and without |
| | measurable disease by FLC: BM plasma cell percentage irrespective of |
| | baseline status (absolute % must be ≥10%) ^{b} |
| | Appearance of new lesion(s), ≥50% increase from nadir in SPD of >1 lesion, or |
| | ≥50% increase in the longest diameter of a previous lesion >1 cm in short axis |
| | ≥50% increase in circulating plasma cells (minimum 200 cells per microliter) if |
| | this is the only measure of disease |
| Clinical | Requires one or more of the following: |
| relapse | Direct indications of increasing disease and/or end organ dysfunction (CRAB |
| | features) ^{f} related to the underlying clonal plasma cell proliferative disorder. It is |
| | not used in calculation of time to progression or PFS but is listed here as |
| | something that can be reported optionally or for use in clinical practice. |
| | Development of new soft tissue plasmacytomas or bone lesions (osteoporotic |
| | fractures do not constitute progression) |
| | Definite increase in the size of existing plasmacytomas or bone lesions. A |
| | definite increase is defined as a 50% (and ≥1 cm) increase as measured |
| | serially by the sum of the products of the cross-diameters of the measurable |
| | lesion. |
| | Hypercalcemia >11 mg/dL (2.65 mmol/L) |
| | Decrease in hemoglobin of ≥2 g/dL (1.25 mmol/L) not related to therapy or |
| | other non-myeloma related conditions |
| | Rise in serum creatinine by 2 mg/dL or more (177 μmol/L or more) from the start |
| | of therapy and attributable to myeloma |
| | Hyperviscosity related to serum paraprotein |
| Relapse | Any one or more of the following: |
| from CR (to | Reappearance of serum or urine M-protein- by immunofixation or |
| be used | electrophoresis |
| only if the | Development of ≥5% plasma cells in the BM |
| endpoint | Appearance of any other sign of progression (i.e., new plasmacytoma, lytic |
| studied is | bone lesion, or hypercalcemia) |
| PFS) ^{c} | |

BM = bone marrow; CR = complete response; CT = computed tomography; FLC = free light chain; M-protein = monoclonal protein; MR = minimal response; MRI = magnetic resonance imaging; PD = progressive disease; PET = positron emission tomography; PFS = progression-free survival; PR = partial response; sCR = stringent complete response; SD = stable disease; SPD = sum of the products of diameters; VGPR = very good partial response.
^{a} Special attention should be given to the emergence of a different M-protein following treatment, especially in the setting of patients having achieved a conventional CR, often related to oligoclonal reconstitution of the immune system. These bands typically disappear over time and in some studies have been associated with a better outcome. Also, appearance of IgGk in patients receiving monoclonal antibodies should be differentiated from the therapeutic antibody.
^{b} In some cases it is possible that the original M-protein light-chain isotype is still detected on immunofixation but the accompanying heavy-chain component has disappeared; this would not be considered a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy-chain isotype (IgG, IgM, etc.). Modified from Durie et al. Leukemia. 20(9): 1467-73 (2006). This requires two consecutive assessments to be carried out at any time before the institution of any new therapy (Durie et al. Leukemia. 29: 2416-7 (2015)).
^{c} Plasmacytoma measurements should be taken from the CT portion of the PET/CT or MRI scans, or dedicated CT scans where applicable. For patients with only skin involvement, the skin lesions should be measured with a ruler. Measurement of tumor size will be determined by the SPD.
^{d} Positive immunofixation alone in a patient previously classified as achieving a CR will not be considered progression. Criteria for relapse from a CR should be used only when calculating disease-free survival.
^{e} In the case where a value is felt to be a spurious result per investigator discretion (e.g. a possible laboratory error), that value will not be considered when determining the lowest value.
^{f} CRAB features = calcium elevation, renal failure, anemia, lytic bone lesions.

A bone marrow biopsy and aspirate are required prior to Cycle 1, Day 1 dosing; at various time points during the study; and at the time of confirmation of CR or at disease progression. The bone marrow sample scheduled prior to Cycle 1, Day 1 may be obtained after the patient's other screening procedures have been completed and enrollment of the patient has been confirmed by the Medical Monitor.

Myeloma-specific tests including serum protein electrophoresis (SPEP) with serum immunofixation electrophoresis (SIFE), SFLCs, and quantitative Ig levels will be conducted at the beginning of every cycle, starting with Cycle 1, Day 1 (screening samples may be used for Cycle 1, Day 1 if drawn within 28 days prior to Cycle 1, Day 1).

Myeloma-specific tests (e.g., a 24-hour urine protein electrophoresis (UPEP) with urine immunofixation and/or electrophoresis (UIFE) for M-protein quantitation) should be at screening and as needed to confirm a response.

The following confirmatory assessments are required for all response categories (sCR, CR, VGPR, PR, and minimal response [MR]):
(a) if extra-medullary disease was previously present, CT scan or MRI with bi-dimensional measurements to confirm reduction in size per IMWG criteria
(b) if extra-medullary disease was previously present, PET-CT scan, CT scan, or MRI to confirm complete resolution
(c) 24-hour UPEP/UIFE (performed locally) to confirm VGPR, even if a UPEP was not performed at screening

To confirm a sCR or CR, SIFE, SFLC, 24-hour UPEP/UIFE, and bone marrow aspiration and biopsy must be performed. Additionally, if extra-medullary disease was previously present, PET-CT scan, CT scan, or MRI to confirm complete resolution.

To confirm progressive disease, the following are required:
(a) if progressive disease is suspected by rising M-protein, SPEP, UPEP, or SFLC analysis should be obtained on two consecutive assessments in two consecutive cycles.
(b) if progressive disease is suspected on development of new bone lesions or soft tissue plasmacytomas or an increase in size of existing bone lesions or soft tissue plasmacytomas, skeletal survey/CT scan/MRI should be obtained and compared with baseline imaging.
(c) if progressive disease is suspected on hypercalcemia attributed solely to MM, local laboratory results levels of serum calcium should be >11 mg/dL and confirmed on a second assessment.

All patients with MM who have clinically suspected extra-medullary disease or known extra-medullary disease at the time of screening must undergo imaging during screening to evaluate for the presence/extent of extramedullary disease. This can be performed by CT scan of the chest, abdomen, and pelvis (preferably with IV contrast if renal function is adequate), PET/CT, or whole-body MRI. Patients who are found to have extra-medullary disease will undergo repeat imaging (preferably the same modality as performed at screening) every 4 cycles (±7 days). Imaging should also be performed upon clinical suspicion of progressive disease or to confirm response. Chest X-ray or ultrasound of the abdomen/liver/spleen may be substituted for CT, PET/CT, or MRI if, per the investigator's assessment, patients are not able to safely tolerate these imaging modalities and the anatomic location of the extramedullary disease is compatible with these alternative imaging methods.

A skeletal survey will be completed at screening and as clinically indicated. The skeletal survey may be completed up to 28 days prior to day 1 of cycle 1. Plain films and CT scans are both acceptable imaging modalities for assessing skeletal disease. Imaging should include the skull, long bones, chest, and pelvis. If plasmacytomas are seen on skeletal survey, bi-dimensional tumor measurements should be recorded. The skeletal survey may be omitted if a PET/CT scan or a low-dose, whole-body CT is performed as part of screening.

### Example 3. Efficacy of an Anti-TIGIT Antagonist Antibody Alone or in Combination with an Anti-CD20 Antibody in Patients with Non-Hodgkin's Lymphoma

To evaluate the efficacy and safety of treatment with an anti-TIGIT antagonist antibody (e.g., an anti-TIGIT antibody disclosed herein (e.g., tiragolumab)) in combination with an anti-CD20 antibody (e.g., rituximab) in patients with a non-Hodgkin's Lymphoma (NHL) (e.g., relapsed or refractory diffuse large B cell lymphoma (DLBCL) or follicular lymphoma (FL)), patients are enrolled in a phase Ia/Ib open-label, global, multicenter study.

Inclusion/Exclusion Criteria

In addition to the inclusion and exclusion criteria disclosed in Example 1, above, patients must also meet the following eligibility requirements.

Patients must have a total hemoglobin ≥9 g/dL without transfusion within 21 days prior to the first dose of the study treatment. Patients who do not meet criteria for hematologic function due to extensive marrow involvement of DLBCL/FL and/or disease-related cytopenias (e.g., immune thrombocytopenia) may be enrolled into the study after discussion with and after approval of the Medical Monitor.

Patients must have serum creatinine≤ULN or estimated creatinine CL≥50 mL/min (calculated or per 24-hr urine collection).

Patients must have a history of histologically-documented DLBCL who have relapsed after or failed to respond to at least two prior systemic treatment regimens (e.g., including at least one prior regimen containing anthracycline, and at least one containing an anti-CD20-directed therapy) and for which no suitable therapy of curative intent or higher priority exists (e.g., standard chemotherapy, autologous SCT).

Patients must also have at least one bi-dimensionally measurable lesion (>1.5 cm in its largest dimension by computerized tomography [CT] scan).

Patients must not have received treatment with radiotherapy within four weeks prior to the first study drug administration; however, are eligible if they (a) have at least one measurable lesion outside of the radiation field or (b) have only one measurable lesion that was previously irradiated but subsequently progressed.

Patients must not either have current or a history of CNS lymphoma or be currently eligible for autologous SCT. Patients must not have uncontrolled hypercalcemia (>1.5 mmol/L ionized calcium or Ca >12 mg/dL or corrected serum calcium ≥ULN) or symptomatic hypercalcemia requiring continued use of bisphosphonate therapy or denosumab. Patients who are receiving bisphosphonate therapy or denosumab specifically to prevent skeletal events and who do not have a history of clinically significant hypercalcemia are eligible. Additionally, patients must not have allergies or hypersensitivities to components of the anti-CD20 antibody (e.g., rituximab) formulation.

Study Treatment Dosage and Administration

Anti-TIGIT Antagonist Antibody as a Single Agent

During treatment, patients receive a fixed dose of 600 mg of an anti-TIGIT antagonist antibody (e.g., an anti-TIGIT antibody disclosed herein, e.g., tiragolumab) administered by intravenous infusion every 3 weeks (q3w) (21±3 days). The anti-TIGIT antagonist antibody (e.g., an anti-TIGIT antibody disclosed herein, e.g., tiragolumab) is administered on Day 1 of each 21-day dosing cycle. Prior to the first infusion of the anti-TIGIT antibody, the patient's vital signs (e.g., pulse rate, respiratory rate, blood pressure, and temperature) are recorded within 60 minutes before starting the infusion. The first infusion of the anti-TIGIT antibody (e.g., an anti-TIGIT antibody disclosed herein, e.g., tiragolumab) is administered over 60 (±10) minutes. During this time, the patient's vital signs (pulse rate, respiratory rate, blood pressure, and temperature) are recorded at 15-minute intervals. Following infusion, the patient is observed for 60 minutes, during which time, the vital signs are monitored at 30 (±10) minutes after the infusion of the anti-TIGIT antagonist antibody).

If no infusion-associated adverse events are experienced during the first infusions of the anti-TIGIT antagonist antibody (e.g., an anti-TIGIT antibody disclosed herein), subsequent infusions can be administered over 30 (±10) minutes. Additionally, the post-infusion observation periods may be reduced to 30 (±10) minutes. Pre-infusion recordation of vital signs shall continue to be recorded within 60 minutes prior to the start of infusion of the anti-TIGIT antagonist antibody (e.g., an anti-TIGIT antibody disclosed herein, e.g., tiragolumab).

If the patient experiences an infusion-related reaction during any infusion of the anti-TIGIT antagonist antibody (e.g., an anti-TIGIT antibody disclosed herein, e.g., tiragolumab), premedication with an antihistamine and/or antipyretic may be administered for subsequent cycles, and the patient's vital signs are to be recorded at 15 (±10) minutes after the infusion.

Anti-TIGIT Antagonist Antibody in Combination with an Anti-CD20 Antibody

During treatment, patients receive a fixed dose of 600 mg of an anti-TIGIT antagonist antibody (e.g., an anti-TIGIT antibody disclosed herein, e.g., tiragolumab) administered by intravenous infusion every 3 weeks (q3w) (21±3 days). The anti-CD20 antibody (e.g., rituximab) is administered by intravenous infusion at a dose of 375 mg/m² weekly (QW) for a total of eight doses. The infusion dose is based on the patient's body surface area at screening and will remain the same throughout the study. Empiric dose adjustments for obese patients (defined as a body mass index of ≥30) may be implemented.

On days where the anti-TIGIT antagonist antibody and the anti-CD20 antibody are administered on the same day, the anti-TIGIT antagonist antibody should be administered first.

The anti-CD20 antibody must be administered to patients in a setting where full emergency resuscitation facilities are immediately available, and patients should remain under close supervision at all times. The infusion of the anti-CD20 antibody may be split over two consecutive days (e.g., 125 mg/m² on Day 1 and 250 mg/m² on Day 2) if the patient is at increased risk for tumor lysis syndrome (e.g., high tumor burden, high peripheral lymphocyte count).

On the days of administration, the anti-TIGIT antagonist antibody is administered as described above. A pre-infusion regimen of oral acetaminophen (e.g., 500 mg) and an antihistamine such as diphenhydramine hydrochloride (25-50 mg) is administered to the patient 30-60 minutes prior to the administration of the anti-CD20 antibody. An additional glucocorticoid (e.g., 100 mg IV prednisone or prednisolone, or equivalent) is allowed at the investigator's discretion. The anti-CD20 antibody is infused at an initial rate of 50 mg/hr. If no IRR or hypersensitivity reaction occurs, the infusion rate may be increased in 50-mg/hr increments every 30 minutes, to a maximum of 400 mg/hr. If an IRR develops, the infusion is slowed or stopped, and infusion reaction medications and supportive care are administered. If the reaction resolves, the infusion may be resumed at a 50% reduction in rate. The patient's vital signs are recorded at 15, 30, 45, and 60 minutes during infusion (±5 minute windows are allowed for all time points). Following infusion, the patient is observed for 60 minutes, during which time the vital signs are monitored at 30 (±10) minutes after the infusion.

Following the first infusion, the IV line or central venous catheter should remain in place for at least 90 minutes in order to administer IV drugs, if necessary. If no adverse events occur after 90 minutes, the IV line may be removed, or the central venous catheter may be de-accessed.

For subsequent infusions, pre-infusion medications are administered and vital signs are recorded as described above. The patient's vital signs are recorded within 60 minutes prior to the infusion of the anti-CD20 antibody. If the patient experienced an IRR or hypersensitivity during the previous infusion of the anti-CD20 antibody, the infusion is administered according to the instructions for the first infusion. If the patient tolerated the prior infusion well (defined as an absence of Grade 2 reactions during a final infusion rate of 100 mg/hr), the initial infusion rate can be 100 mg/hr. If no IRR occurs, the infusion rate may be increased in 100-mg/hr increments every 30 minutes, to a maximum of 400 mg/hr.

If an IRR develops, the infusion is slowed or stopped, and infusion reaction medications and supportive care are administered. If the reaction resolves, the infusion may be resumed at a 50% reduction in rate.

If the patient tolerated the first or a subsequent infusion of the anti-CD20 antibody (without premedication) well without IRR, the observation after the next and following infusions may be reduced to 30 minutes; otherwise, the observation period should remain 60 minutes. The patients vital signs may be recorded at 15 (±10) minutes after the infusion. Following the second and any subsequent infusions, the IV line or central venous catheter should remain in place for at least 30 minutes in order to administer IV drugs, if necessary. If no adverse events occur after 30 minutes, the IV line may be removed, or the central venous catheter may be de-accessed.

Activity Analyses

The activity of the anti-TIGIT antagonist antibody as a single agent or in combination with the anti-CD20 antibody will be evaluated on the basis of ORR, DOR, PFS, and OS. For R/R DLBCL or R/R FL, ORR is defined as the proportion of patients with a CR or PR on two consecutive occasions ≥4 weeks apart, according to the Lugano classification, as described in Cheson et al. J. Clin. Oncol. 32(27):3059-3067 (2014), and in Table 4. ORR may be assessed during screening, during each of cycles 1-21, and upon discontinuation of study treatment. DOR is defined as the time from the first occurrence of a documented objective response to disease progression or death from any cause during the study (defined as within 30 days after the last dose of study drug) (whichever occurs first), according to the Lugano classification. PFS is defined as the time from enrollment to the first occurrence of disease progression or death from any cause during the study (defined as within 30 days after the last dose of study drug) (whichever occurs first), according to the Lugano classification.

Up to six of the largest target nodes, nodal masses, or other lymphomatous lesions that are measurable in two diameters should be identified from different body regions representative of the patient's overall disease burden and include mediastinal and retroperitoneal disease, if involved. At baseline, a measurable node must be greater than 15 mm in longest diameter (LDi). Measurable extranodal disease may be included in the six representative, measured lesions. At baseline, measurable extranodal lesions should be greater than 10 mm LDi.

All other lesions (including nodal, extranodal, and assessable disease) should be followed as non-measured disease as non-target lesions (e.g. cutaneous, GI, bone, spleen, liver, kidneys, pleural or pericardial effusions, ascites, bone, bone marrow).

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate progressive disease. The LDi and smallest diameter (SDi) are no longer needed to determine progression.

TABLE 4
Lugano Response Criteria for Malignant Lymphoma

| Response and Site | PET-CT-Based Response | CT-Based Response |
|---|---|---|
| Complete Response | Complete metabolic response | Complete radiologic response (all of |
| | | the following) |
| Lymph nodes and | Score 1, 2, or 3 ^{a} with or without a | Target nodes/nodal masses must |
| extralymphatic | residual mass on 5PS ^{b} | regress to ≤1.5 cm in LDi |
| sites | | No extralymphatic sites of disease |
| | It is recognized that in Waldeyer's ring | |
| | or extranodal sites with high | |
| | physiologic uptake or with activation | |
| | within spleen or marrow (e.g., with | |
| | chemotherapy or myeloid colony- | |
| | stimulating factors), uptake may be | |
| | greater than normal mediastinum | |
| | and/or liver. In this circumstance, | |
| | complete metabolic response may | |
| | be inferred if uptake at sites of initial | |
| | involvement is no greater than | |
| | surrounding normal tissue even if | |
| | the tissue has high physiologic | |
| | uptake. | |
| Non-measured | Not applicable | Absent |
| lesion | | |
| Organ | Not applicable | Regress to normal |
| enlargement | | |
| New lesions | None | None |
| Bone marrow | No evidence of FDG-avid disease in | Normal by morphology; if |
| | marrow | indeterminate, IHC negative |
| Partial Response | Partial metabolic response | Partial remission (all of the |
| | | following) |
| Lymph nodes and | Score 4 or 5 ^{b} with reduced uptake | ≥50% decrease in SPD of up to 6 |
| extralymphatic | compared with baseline and residual | target measurable nodes and |
| sites | mass(es) of any size | extranodal sites |
| | At interim, these findings suggest | When a lesion is too small to |
| | responding disease | measure on CT, assign 5 mm × 5 |
| | | mm as the default value |
| | At end of treatment, these findings | When no longer visible, 0 × 0 mm |
| | indicate residual disease | For a node >5 mm × 5 mm, but |
| | | smaller than normal, use actual |
| | | measurement for calculation |
| Non-measured | Not applicable | Absent/normal, regressed, but no |
| lesion | | increase |
| Organ | Not applicable | Spleen must have regressed by |
| enlargement | | >50% in length beyond normal |
| New lesions | None | None |
| Bone marrow | Residual uptake higher than uptake in | Not applicable |
| | normal marrow but reduced compared | |
| | with baseline (diffuse uptake | |
| | compatible with reactive changes from | |
| | chemotherapy allowed). If there are | |
| | persistent focal changes in the marrow | |
| | in the context of a nodal response, | |
| | consideration should be given to | |
| | further evaluation with MRI or biopsy | |
| | or an interval scan. | |
| No response or | No metabolic response | Stable disease |
| stable disease | | |
| Target | Score 4 or 5 ^{b} with no significant | <50% decrease from baseline in |
| nodes/nodal | change in FDG uptake from baseline | SPD of up to 6 dominant, |
| masses, | at interim or end of treatment | measurable nodes and extranodal |
| extranodal lesions | | sites; no criteria for progressive |
| | | disease are met |
| Non-measured | Not applicable | No increase consistent with |
| lesion | | progression |
| Organ | Not applicable | No increase consistent with |
| enlargement | | progression |
| New lesions | None | None |
| Bone marrow | No change from baseline | Not applicable |
| Progressive | Progressive metabolic disease | Progressive disease requires at |
| disease | | least 1 of the following |
| Individual target | Score 4 or 5 ^{b} with an increase in | PPD progression: |
| nodes/nodal | intensity of uptake from baseline | |
| masses | and/or | |
| Extranodal lesions | New FDG-avid foci consistent with | An individual node/lesion must be |
| | lymphoma at interim or end-of- | abnormal with: |
| | treatment assessment | LDi >1.5 cm and |
| | | Increase by ≥50% from PPD nadir and |
| | | An increase in LDi or SDi from nadir |
| | | 0.5 cm for lesions ≤2 cm |
| | | 1.0 cm for lesions >2 cm |
| | | In the setting of splenomegaly (>13 |
| | | cm), the splenic length must increase |
| | | by >50% of the extent of its prior |
| | | increase beyond baseline (e.g., a 15- |
| | | cm spleen must increase to >16 cm). |
| | | If no prior splenomegaly, must |
| | | increase by at least 2 cm from |
| | | baseline. |
| | | New or recurrent splenomegaly |
| Non-measured | None | New or clear progression of |
| lesions | | preexisting non-measured lesions |
| New lesions | New FDG-avid foci consistent with | Regrowth of previously resolved |
| | lymphoma rather than another etiology | lesions |
| | (e.g., infection, inflammation); if | A new node >1.5 cm in any axis |
| | uncertain regarding etiology of new | A new extranodal site >1.0 cm in any |
| | lesions, biopsy or interval scan may be | axis; if <1.0 cm in any axis, its |
| | considered | presence must be unequivocal and |
| | | must be attributable to lymphoma |
| | | Assessable disease of any size |
| | | unequivocally attributable to |
| | | lymphoma |
| Bone marrow | New or recurrent FDG-avid foci | New or recurrent involvement |

5PS = 5-point scale; CCT = computed tomography; FDG = fluorodeoxyglucose; IHC = immunohistochemistry; LDi = longest transverse diameter of a lesion; MRI = magnetic resonance imaging; PET = positron emission tomography; PPD = cross product of the LDi and perpendicular diameter; SDi = shortest axis perpendicular to the LDi; SPD = sum of the product of the perpendicular diameters for multiple lesions.
^{a} A score of 3 in many patients indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those in solid organs (e.g., liver, spleen, kidneys, and lungs), gastrointestinal involvement, cutaneous lesions, or those noted on palpation. Non-measured lesions: Any disease not selected as measured; dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{b} PET 5PS: 1 = no uptake above background; 2 = uptake mediastinum; 3 = uptake > mediastinum but ≤ liver; 4 = uptake moderately > liver; 5 = uptake markedly higher than liver and/or new lesions; X = new areas of uptake unlikely to be related to lymphoma.

FDG PET/CT imaging should be performed to assess FDG-avid lymphomas and to assess baseline tumor burden in this study. For lymphomas that are shown to not be FDG-avid or have variable FDG uptake, conventional CT scans may be performed. Following the initial PET/CT scan, PET/CT scans may be limited to areas of disease involvement if required by local health authorities.

CT scans should be performed with contiguous cuts of ≤10 mm in slice thickness and with resolution sufficient to allow accurate and consistent comparison of target lesion measurements with serial scans. CT scans with oral and IV contrast should include chest, abdomen, and pelvic scans; CT scans of the neck should be included if clinically indicated. Oral contrast may be omitted per institutional standards. CT scans for response assessment may be limited to areas of prior involvement only if required by local health authorities. At the investigator's discretion, PET/CT or CT scans may be repeated at any time if PD is suspected. MRI scans may be used instead of CT scans in patients for whom they are contraindicated.

If contrast is contraindicated (e.g., in patients with contrast allergy or impaired renal function), CT or combined PET/CT scans without contrast are permitted provided they permit consistent and precise measurement of target lesions during the study treatment period.

The same radiographic assessment modality should be used for all response evaluations, in order to ensure consistency across different timepoints (e.g., PET/CT with the same contrast protocol for CT scans). A full radiographic assessment must be performed any time disease progression or relapse is suspected. For patients who undergo screening/post-treatment biopsies, these lesions may not be selected as target lesions.

Bone marrow examinations including both biopsy and aspirate for morphology (flow studies are optional) are required at screening for staging purposes unless a bone marrow examination was done following evidence of relapse and within 3 months prior to Cycle 1, Day 1. For patients with DLBCL at both initial diagnosis and study entry, screening PET scan can be utilized to assess bone marrow involvement and bone marrow examinations are not required unless clinically indicated (as described in Cheson et al. J. Clin. Oncol. 32(27):3059-3067 (2014)). If bone marrow is positive for tumor at screening, a repeat bone marrow examination is performed to confirm a radiologic assessment of CR. If bone marrow assessment was negative at baseline and there is no radiographic evidence of progression for lymphoma patients, an additional analysis may be performed to provide evidence of relapse.

For patients with more than one bi-dimensionally measurable lesion (>1.5 cm in the largest dimension by CT scan), tumor biopsies from safely accessible tumor sites (i.e., without unacceptable risk of major procedural complication[s] per investigator assessment) are required prior to Cycle 1, Day 1 dosing; between Cycle 1, Day 15 and Cycle 2, Day 1; and at disease progression.

### VIII. Other Embodiments

Some embodiments of the technology described herein can be defined according to any of the following numerated embodiments:

1. A method for treating a subject having a hematologic cancer, the method comprising administering to the subject an effective amount of an anti-TIGIT antagonist antibody and an effective amount of an anti-CD38 antibody.

2. The method of embodiment 1, wherein the anti-TIGIT antagonist antibody is administered prior to the anti-CD38 antibody.

3. The method of embodiment 2, wherein the method comprises a first observation period following administration of the anti-TIGIT antagonist antibody and a second observation period following administration of the anti-CD38 antibody.

4. The method of embodiment 3, wherein the first observation period and the second observation period are each between about 30 minutes to about 60 minutes in length.

5. The method of embodiment 1, wherein the anti-CD38 antibody is administered prior to the anti-TIGIT antagonist antibody.

6. The method of embodiment 5, wherein the method comprises a first observation period following administration of the anti-CD38 antibody and a second observation period following administration of the anti-TIGIT antagonist antibody.

7. The method of embodiment 6, wherein the first observation period and the second observation period are each between about 30 minutes to about 60 minutes in length.

8. The method of any one of embodiments 1-7, further comprising administering to the subject a corticosteroid prior to the administration of the anti-CD38 antibody.

9. The method of any one of embodiments 1-8, further comprising administering to the subject an antipyretic prior to the administration of the anti-CD38 antibody.

10. The method of any one of embodiments 1-9, further comprising administering to the subject an antihistamine prior to the administration of the anti-CD38 antibody.

11. The method of any one of embodiments 1-10, further comprising administering to the subject a corticosteroid, an antipyretic, and an antihistamine prior to the administration of the anti-CD38 antibody.

12. The method of embodiment 11, wherein the corticosteroid is methylprednisolone, the antipyretic is acetaminophen, and/or the antihistamine is diphenhydramine.

13. The method of any one of embodiments 1-12, wherein the method comprises administering to the subject a corticosteroid on each of the two days following the administration of the anti-CD38 antibody.

14. The method of any one of embodiments 1-13, wherein the method comprises administering to the subject the anti-CD38 antibody at a dose of about 16 mg/kg.

15. The method of any one of embodiments 1-14, wherein the anti-CD38 antibody is an anti-CD38 antagonist antibody.

16. The method of any one of embodiments 1-15, wherein the anti-CD38 antibody comprises the following complementarity determining regions (CDRs):

(a) a CDR-H1 comprising the amino acid sequence of SFAMS (SEQ ID NO: 20);

(b) a CDR-H2 comprising the amino acid sequence of AISGSGGGTYYADSVKG (SEQ ID NO: 21);

(c) a CDR-H3 comprising the amino acid sequence of DKILWFGEPVFDY (SEQ ID NO: 22);

(d) a CDR-L1 comprising the amino acid sequence of RASQSVSSYLA (SEQ ID NO: 23);

(e) a CDR-L2 comprising the amino acid sequence of DASNRAT (SEQ ID NO: 24); and

(f) a CDR-L3 comprising the amino acid sequence of QQRSNWPPTF (SEQ ID NO: 25).

17. The method of embodiment 16, wherein the anti-CD38 antibody further comprises the following light chain variable region framework regions (FRs):

(a) an FR-L1 comprising the amino acid sequence of EIVLTQSPATLSLSPGERATLSC (SEQ ID NO: 26);

(b) an FR-L2 comprising the amino acid sequence of WYQQKPGQAPRLLIY (SEQ ID NO: 27);

(c) an FR-L3 comprising the amino acid sequence of GIPARFSGSGSGTDFTLTISSLEPEDFAVYYC (SEQ ID NO: 28); and

(d) an FR-L4 comprising the amino acid sequence of GQGTKVEIK (SEQ ID NO: 29).

18. The method of embodiment 17, wherein the anti-CD38 antibody further comprises the following heavy chain variable region FRs:

(a) an FR-H1 comprising the amino acid sequence of

(SEQ ID NO: 30)
EVQLLESGGGLVQPGGSLRLSCAVSGFTFN;

(b) an FR-H2 comprising the amino acid sequence of WVRQAPGKGLEWVS (SEQ ID NO: 31);

(c) an FR-H3 comprising the amino acid sequence of RFTISRDNSKNTLYLQMNSLRAEDTAVYFCAK (SEQ ID NO: 32); and

(d) an FR-H4 comprising the amino acid sequence of WGQGTLVTVSS (SEQ ID NO: 33).

19. The method of any one of embodiments 16-18, wherein the anti-CD38 antibody further comprises:

(a) a heavy chain variable (VH) domain comprising an amino acid sequence having at least 95% sequence identity to the amino acid sequence of

(SEQ ID NO: 34)
EVQLLESGGGLVQPGGSLRLSCAVSGFTFNSFAMSWVRQAPGKGLEWVSAI
SGSGGGTYYADSVKGRFTISRDNSKNTLYLQMNSLRAEDTAVYFCAKDKIL
WFGEPVFDYWGQGTLVTVSS;

(b) a light chain variable (VL) domain comprising an amino acid sequence having at least 95% sequence identity to the amino acid sequence of EIVLTQSPATLSLSPGERATLSCRASQSVSSYLAWYQQKPGQAPRLLIYDASNRATGIP ARFSGSGSGTDFTLTISSLEPEDFAVYYCQQRSNWPPTFGQGTKVEIK (SEQ ID NO: 35); or

(c) a VH domain as in (a) and a VL domain as in (b).

20. The method of embodiment 19, wherein the anti-CD38 antibody comprises:

(a) a VH domain comprising the amino acid sequence of SEQ ID NO: 34; and

(b) a VL domain comprising the amino acid sequence of SEQ ID NO: 35.

21. The method of any one of embodiments 1-20, wherein the anti-CD38 antibody is a monoclonal antibody.

22. The method of any one of embodiments 1-21, wherein the anti-CD38 antibody is a human antibody.

23. The method of any one of embodiments 1-21, wherein the anti-CD38 antibody is a full-length antibody.

24. The method of any one of embodiments 1-23, wherein the anti-CD38 antibody is daratumumab.

25. The method of any one of embodiments 1-24, wherein the anti-CD38 antibody is an antibody fragment that binds CD38 selected from the group consisting of Fab, Fab′, Fab′-SH, Fv, single chain variable fragment (scFv), and (Fab′)₂ fragments.

26. The method of any one of embodiments 1-25, wherein the anti-CD38 antibody is an IgG class antibody.

27. The method of embodiment 26, wherein the IgG class antibody is an IgG1 subclass antibody.

28. The method of any one of embodiments 1-27, wherein the method comprises administering to the subject the anti-CD38 antibody intravenously.

29. The method of any one of embodiments 1-28, wherein the hematologic cancer is a myeloma.

30. The method of embodiment 29, wherein the myeloma is a multiple myeloma (MM).

31. The method of embodiment 30, wherein the MM is a relapsed or refractory MM.

32. A method for treating a subject having a hematological cancer, the method comprising administering to the subject an effective amount of an anti-TIGIT antagonist antibody and an effective amount of an anti-CD20 antibody.

33. The method of embodiment 32, wherein the anti-TIGIT antagonist antibody is administered prior to the anti-CD20 antibody.

34. The method of embodiment 32 or 33, wherein the method comprises a first observation period following administration of the anti-TIGIT antagonist antibody and a second observation period following administration of the anti-CD20 antibody.

35. The method of embodiment 34, wherein the first observation period and the second observation period are each between about 30 minutes to about 60 minutes in length.

36. The method of any one of embodiments 32-35, wherein the subject has an infusion-related reaction (IRR) to the anti-TIGIT antagonist antibody, and the method further comprises administering to the subject an antihistamine and/or an antipyretic prior to a subsequent administration of the anti-TIGIT antagonist antibody.

37. The method of any one of embodiments 32-36, further comprising administering to the subject an antipyretic and an antihistamine prior to each administration of the anti-CD20 antibody.

38. The method of embodiment 37, wherein the antipyretic is acetaminophen and the antihistamine is diphenhydramine.

39. The method of embodiment 38, further comprising administering to the subject a glucocorticoid prior to each administration of the anti-CD20 antibody.

40. The method of any one of embodiments 32-39, wherein the method comprises administering to the subject the anti-CD20 antibody at a dose of about 375 mg/m².

41. The method of any one of embodiments 32-40, wherein the anti-CD20 antibody is an anti-CD20 antagonist antibody.

42. The method of any one of embodiments 32-41, wherein the anti-CD20 antibody comprises the following CDRs:

(a) a CDR-H1 comprising the amino acid sequence of SYNMH (SEQ ID NO: 36);

(b) a CDR-H2 comprising the amino acid sequence of AIYPGNGDTSYNQKFKG (SEQ ID NO: 37);

(c) a CDR-H3 comprising the amino acid sequence of STYYGGDWYFNV (SEQ ID NO: 38);

(d) a CDR-L1 comprising the amino acid sequence of RASSSVSYIH (SEQ ID NO: 39);

(e) a CDR-L2 comprising the amino acid sequence of ATSNLAS (SEQ ID NO: 40); and

(f) a CDR-L3 comprising the amino acid sequence of QQWTSNPPT (SEQ ID NO: 41).

43. The method of embodiment 42, wherein the anti-CD20 antibody further comprises the following light chain variable region FRs:

(a) an FR-L1 comprising the amino acid sequence of QIVLSQSPAILSASPGEKVTMTC (SEQ ID NO: 42);

(b) an FR-L2 comprising the amino acid sequence of WFQQKPGSSPKPWIY (SEQ ID NO: 43);

(c) an FR-L3 comprising the amino acid sequence of GVPVRFSGSGSGTSYSLTISRVEAEDAATYYC (SEQ ID NO: 44); and

(d) an FR-L4 comprising the amino acid sequence of FGGGTKLEIK (SEQ ID NO: 45).

44. The method of embodiment 43, wherein the anti-CD20 antibody further comprises the following heavy chain variable region FRs:

(a) an FR-H1 comprising the amino acid sequence of

(SEQ ID NO: 46)
QVQLQQPGAELVKPGASVKMSCKASGYTFT;

(b) an FR-H2 comprising the amino acid sequence of WVKQTPGRGLEWIG (SEQ ID NO: 47);

(c) an FR-H3 comprising the amino acid sequence of KATLTADKSSSTAYMQLSSLTSEDSAVYYCAR (SEQ ID NO: 48); and

(d) an FR-H4 comprising the amino acid sequence of WGAGTTVTVS (SEQ ID NO: 49).

45. The method of any one of embodiments 44-44, wherein the anti-CD20 antibody further comprises:

(a) a VH domain comprising an amino acid sequence having at least 95% sequence identity to the amino acid sequence of

(SEQ ID NO: 50)
QVQLQQPGAELVKPGASVKMSCKASGYTFTSYNMHWVKQTPGRGLEWIGAI
YPGNGDTSYNQKFKGKATLTADKSSSTAYMQLSSLTSEDSAVYYCARSTYY
GGDWYFNVWGAGTTVTVS;

(b) a VL domain comprising an amino acid sequence having at least 95% sequence identity to the amino acid sequence of QIVLSQSPAILSASPGEKVTMTCRASSSVSYIHWFQQKPGSSPKPWIYATSNLASGVPVRFSGSGSG TSYSLTISRVEAEDAATYYCQQWTSNPPTFGGGTKLEIK (SEQ ID NO: 51); or

(c) a VH domain as in (a) and a VL domain as in (b).

46. The method of embodiment 45, wherein the anti-CD20 antibody comprises:

(a) a VH domain comprising the amino acid sequence of SEQ ID NO:50; and

(b) a VL domain comprising the amino acid sequence of SEQ ID NO:51.

47. The method of any one of embodiments 32-46, wherein the anti-CD20 antibody is a monoclonal antibody.

48. The method of any one of embodiments 32-47, wherein the anti-CD20 antibody is a chimeric antibody.

49. The method of any one of embodiments 32-48, wherein the anti-CD20 antibody is a full-length antibody.

50. The method of any one of embodiments 32-49, wherein the anti-CD20 antibody is rituximab.

51. The method of any one of embodiments 32-48, wherein the anti-CD20 antibody is an antibody fragment that binds CD20 selected from the group consisting of Fab, Fab′, Fab′-SH, Fv, single chain variable fragment (scFv), and (Fab′)₂ fragments.

52. The method of any one of embodiments 32-51, wherein the anti-CD20 antibody is an IgG class antibody.

53. The method of embodiment 52, wherein the IgG class antibody is an IgG1 subclass antibody.

54. The method of any one of embodiments 32-53, wherein the method comprises administering to the subject the anti-CD20 antibody intravenously.

55. The method of any one of embodiments 32-54, wherein the hematologic cancer is a lymphoma.

56. The method of embodiment 55, wherein the lymphoma is a non-Hodgkin's lymphoma (NHL).

57. The method of embodiment 56, wherein the NHL is a relapsed or refractory diffuse large B cell lymphoma (DLBCL).

58. The method of embodiment 56, wherein the NHL is a relapsed or refractory follicular lymphoma (FL).

59. A method for treating a subject having a hematologic cancer, the method comprising administering to the subject an anti-TIGIT antagonist antibody at a fixed dose of between about 30 mg to about 1200 mg and an anti-CD38 antibody at a dose of between about 8 mg/kg to about 24 mg/kg in a dosing regimen comprising at least nine dosing cycles, wherein:

(a) the anti-TIGIT antagonist antibody is administered once every three weeks; and

(b) the anti-CD38 antibody is administered once every week during each of dosing cycles 1-3, once every three weeks during each of dosing cycles 4-8, and once every four weeks beginning on dosing cycle 9.

60. The method of embodiment 59, wherein the length of each dosing cycle is 21 days.

61. The method of embodiment 59 or 60, wherein the anti-TIGIT antagonist antibody is administered on or about day 1 of each dosing cycle.

62. The method of any one of embodiments 59-61, wherein the anti-CD38 antibody is administered on or about days 1, 8, and 15 of each of dosing cycles 1-3, on or about day 1 of each of dosing cycles 4-8, and on or about day 1 dosing cycle 9.

63. The method of any one of embodiments 59-62, wherein the anti-TIGIT antagonist antibody and the anti-CD38 antibody are both administered on or about day 1 of each of dosing cycles 1-9.

64. The method of embodiment 63, wherein the anti-TIGIT antagonist antibody is administered prior to the anti-CD38 antibody.

65. The method of embodiment 64, wherein the method comprises a first observation period following administration of the anti-TIGIT antagonist antibody and a second observation period following administration of the anti-CD38 antibody.

66. The method of embodiment 65, wherein the first observation period and the second observation period are each between about 30 minutes to about 60 minutes in length.

67. The method of embodiment 63, wherein the anti-CD38 antibody is administered prior to the anti-TIGIT antagonist antibody.

68. The method of embodiment 67, wherein the method comprises a first observation period following administration of the anti-CD38 antibody and a second observation period following administration of the anti-TIGIT antagonist antibody.

69. The method of embodiment 68, wherein the first observation period and the second observation period are each between about 30 minutes to about 60 minutes in length.

70. The method of any one of embodiments 59-69, wherein the dosing regimen comprises at least 12 dosing cycles.

71. The method of embodiment 70, wherein the dosing regimen comprises at least 16 dosing cycles.

72. The method of any one of embodiments 59-71, further comprising administering to the subject a corticosteroid prior to each administration of the anti-CD38 antibody.

73. The method of any one of embodiments 59-72, further comprising administering to the subject an antipyretic prior to each administration of the anti-CD38 antibody.

74. The method of any one of embodiments 59-73, further comprising administering to the subject an antihistamine prior to each administration of the anti-CD38 antibody.

75. The method of any one of embodiments 59-74, further comprising administering to the subject a corticosteroid, an antipyretic, and an antihistamine prior to each administration of the anti-CD38 antibody.

76. The method of any one of embodiments 72-75, wherein the corticosteroid is methylprednisolone, the antipyretic is acetaminophen, and/or the antihistamine is diphenhydramine.

77. The method of any one of embodiments 59-76, wherein the method comprises administering to the subject a corticosteroid on each of the two days following administration of the anti-CD38 antibody.

78. The method of any one of embodiments 59-77, wherein the method comprises administering to the subject the anti-CD38 antibody at a dose of about 16 mg/kg.

79. The method of any one of embodiments 59-78, wherein the anti-CD38 antibody is an anti-CD38 antagonist antibody.

80. The method of any one of embodiments 59-79, wherein the anti-CD38 antibody comprises the following complementarity determining regions (CDRs):

(a) a CDR-H1 comprising the amino acid sequence of SFAMS (SEQ ID NO: 20);

(b) a CDR-H2 comprising the amino acid sequence of AISGSGGGTYYADSVKG (SEQ ID NO: 21);

(c) a CDR-H3 comprising the amino acid sequence of DKILWFGEPVFDY (SEQ ID NO: 22);

(d) a CDR-L1 comprising the amino acid sequence of RASQSVSSYLA (SEQ ID NO: 23);

(e) a CDR-L2 comprising the amino acid sequence of DASNRAT (SEQ ID NO: 24); and

(f) a CDR-L3 comprising the amino acid sequence of QQRSNWPPTF (SEQ ID NO: 25).

81. The method of embodiment 80, wherein the anti-CD38 antibody further comprises the following light chain variable region framework regions (FRs):

(a) an FR-L1 comprising the amino acid sequence of EIVLTQSPATLSLSPGERATLSC (SEQ ID NO: 26);

(b) an FR-L2 comprising the amino acid sequence of WYQQKPGQAPRLLIY (SEQ ID NO: 27);

(c) an FR-L3 comprising the amino acid sequence of GIPARFSGSGSGTDFTLTISSLEPEDFAVYYC (SEQ ID NO: 28); and

(d) an FR-L4 comprising the amino acid sequence of GQGTKVEIK (SEQ ID NO: 29).

82. The method of embodiment 81, wherein the anti-CD38 antibody further comprises the following heavy chain variable region FRs:

(a) an FR-H1 comprising the amino acid sequence of

(SEQ ID NO: 30)
EVQLLESGGGLVQPGGSLRLSCAVSGFTFN;

(b) an FR-H2 comprising the amino acid sequence of WVRQAPGKGLEWVS (SEQ ID NO: 31);

(c) an FR-H3 comprising the amino acid sequence of RFTISRDNSKNTLYLQMNSLRAEDTAVYFCAK (SEQ ID NO: 32); and

(d) an FR-H4 comprising the amino acid sequence of WGQGTLVTVSS (SEQ ID NO: 33).

83. The method of any one of embodiments 80-82, wherein the anti-CD38 antibody further comprises:

(a) a heavy chain variable (VH) domain comprising an amino acid sequence having at least 95% sequence identity to the amino acid sequence of

(SEQ ID NO: 34)
EVQLLESGGGLVQPGGSLRLSCAVSGFTFNSFAMSWVRQAPGKGLEWVSAI
SGSGGGTYYADSVKGRFTISRDNSKNTLYLQMNSLRAEDTAVYFCAKDKIL
WFGEPVFDYWGQGTLVTVSS;

(b) a light chain variable (VL) domain comprising an amino acid sequence having at least 95% sequence identity to the amino acid sequence of EIVLTQSPATLSLSPGERATLSCRASQSVSSYLAWYQQKPGQAPRLLIYDASNRATGIP ARFSGSGSGTDFTLTISSLEPEDFAVYYCQQRSNWPPTFGQGTKVEIK (SEQ ID NO: 35); or

(c) a VH domain as in (a) and a VL domain as in (b).

84. The method of embodiment 83, wherein the anti-CD38 antibody comprises:

(a) a VH domain comprising the amino acid sequence of SEQ ID NO: 34; and

(b) a VL domain comprising the amino acid sequence of SEQ ID NO: 35.

85. The method of any one of embodiments 59-84, wherein the anti-CD38 antibody is a monoclonal antibody.

86. The method of any one of embodiments 59-85, wherein the anti-CD38 antibody is a human antibody.

87. The method of any one of embodiments 59-86, wherein the anti-CD38 antibody is a full-length antibody.

88. The method of any one of embodiments 59-87, wherein the anti-CD38 antibody is daratumumab.

89. The method of any one of embodiments 59-86, wherein the anti-CD38 antibody is an antibody fragment that binds CD38 selected from the group consisting of Fab, Fab′, Fab′-SH, Fv, single chain variable fragment (scFv), and (Fab′)₂ fragments.

90. The method of any one of embodiments 59-89, wherein the anti-CD38 antibody is an IgG class antibody.

91. The method of embodiment 90, wherein the IgG class antibody is an IgG1 subclass antibody.

92. The method of any one of embodiments 59-91, wherein the method comprises administering to the subject the anti-CD38 antibody intravenously.

93. The method of any one of embodiments 59-92, wherein the hematologic cancer is a myeloma.

94. The method of embodiment 93, wherein the myeloma is a multiple myeloma (MM).

95. The method of embodiment 94, wherein the MM is a relapsed or refractory MM.

96. A method for treating a subject having a hematologic cancer, the method comprising administering to the subject an anti-TIGIT antagonist antibody at a fixed dose of between about 30 mg to about 1200 mg and an anti-CD20 antibody at a dose of between about 250 mg/m² to about 500 mg/m² in a dosing regimen comprising at least a first, a second, and a third dosing cycle, wherein:

(a) the anti-TIGIT antagonist antibody is administered once every three weeks; and

(b) the anti-CD20 antibody is administered once every week.

97. The method of embodiment 96, wherein:

(a) each dosing cycle of the dosing regimen comprises a single dose of the anti-TIGIT antagonist antibody;

(b) the first dosing cycle comprises a first dose (C1D1), a second dose (C1D2), and a third dose (C1D3) of the anti-CD20 antibody;

(c) the second dosing cycle comprises a first dose (C2D1), a second dose (C2D2), and a third dose (C2D3) of the anti-CD20 antibody; and

(d) the third dosing cycle comprises at least a first dose (C3D1) and a second dose (C3D2) of the anti-CD20 antibody.

98. The method of embodiment 96 or 97, wherein the dosing regimen comprises a total of eight doses of the anti-CD20 antibody.

99. The method of any one of embodiments 96-98, wherein the length of each dosing cycle is 21 days.

100. The method of any one of embodiments 96-99, wherein the method comprises administering to the subject the anti-TIGIT antagonist antibody on or about day 1 of each dosing cycle.

101. The method of any one of embodiments 96-100, wherein the method comprises administering to the subject the C1 D1, the C1 D2, and the C1 D3 of the anti-CD20 antibody on or about days 1, 8, and 15, respectively, of the first dosing cycle.

102. The method of any one of embodiments 96-101, wherein the method comprises administering to the subject the C2D1, the C2D2, and the C2D3 of the anti-CD20 antibody on or about days 1, 8, and 15, respectively, of the second dosing cycle.

103. The method of any one of embodiments 96-102, wherein the method comprises administering to the subject the C3D1 and the C3D2 of the anti-CD20 antibody on or about days 1 and 8, respectively, of the third dosing cycle.

104. The method of any one of embodiments 96-103, wherein the anti-TIGIT antagonist antibody and the anti-CD20 antibody are both administered on or about day 1 of each of dosing cycles 1, 2, and 3.

105. The method of embodiment 104, wherein the anti-TIGIT antagonist antibody is administered prior to the anti-CD20 antibody.

106. The method of embodiment 104 or 105, wherein the method comprises a first observation period following administration of the anti-TIGIT antagonist antibody and a second observation period following administration of the anti-CD20 antibody.

107. The method of embodiment 106, wherein the first observation period and the second observation period are each between about 30 minutes to about 60 minutes in length.

108. The method of any one of embodiments 96-107, wherein the dosing regimen comprises at least 12 dosing cycles.

109. The method of embodiment 108, wherein the dosing regimen comprises at least 16 dosing cycles.

110. The method of any one of embodiments 96-109, wherein the subject has an infusion-related reaction (IRR) to the anti-TIGIT antagonist antibody, and the method further comprises administering to the subject an antihistamine and/or an antipyretic prior to a subsequent administration of the anti-TIGIT antagonist antibody.

111. The method of any one of embodiments 96-110, further comprising administering to the subject an antipyretic and an antihistamine prior to each administration of the anti-CD20 antibody.

112. The method of embodiment 111, wherein the antipyretic is acetaminophen and the antihistamine is diphenhydramine.

113. The method of embodiment 112, further comprising administering to the subject a glucocorticoid prior to each administration of the anti-CD20 antibody.

114. The method of any one of embodiments 96-113, wherein the method comprises administering to the subject the anti-CD20 antibody at a dose of about 375 mg/m².

115. The method of any one of embodiments 96-114, wherein the anti-CD20 antibody is an anti-CD20 antagonist antibody.

116. The method of any one of embodiments 96-115, wherein the anti-CD20 antibody comprises the following CDRs:

(a) a CDR-H1 comprising the amino acid sequence of SYNMH (SEQ ID NO: 36);

(b) a CDR-H2 comprising the amino acid sequence of AIYPGNGDTSYNQKFKG (SEQ ID NO: 37);

(c) a CDR-H3 comprising the amino acid sequence of STYYGGDWYFNV (SEQ ID NO: 38);

(d) a CDR-L1 comprising the amino acid sequence of RASSSVSYIH (SEQ ID NO: 39);

(e) a CDR-L2 comprising the amino acid sequence of ATSNLAS (SEQ ID NO: 40); and

(f) a CDR-L3 comprising the amino acid sequence of QQWTSNPPT (SEQ ID NO: 41).

117. The method of embodiment 116, wherein the anti-CD20 antibody further comprises the following light chain variable region FRs:

(a) an FR-L1 comprising the amino acid sequence of QIVLSQSPAILSASPGEKVTMTC (SEQ ID NO: 42);

(b) an FR-L2 comprising the amino acid sequence of WFQQKPGSSPKPWIY (SEQ ID NO: 43);

(c) an FR-L3 comprising the amino acid sequence of GVPVRFSGSGSGTSYSLTISRVEAEDAATYYC (SEQ ID NO: 44); and

(d) an FR-L4 comprising the amino acid sequence of FGGGTKLEIK (SEQ ID NO: 45).

118. The method of embodiment 117, wherein the anti-CD20 antibody further comprises the following heavy chain variable region FRs:

(a) an FR-H1 comprising the amino acid sequence of

(SEQ ID NO: 46)
QVQLQQPGAELVKPGASVKMSCKASGYTFT;

(b) an FR-H2 comprising the amino acid sequence of WVKQTPGRGLEWIG (SEQ ID NO: 47);

(c) an FR-H3 comprising the amino acid sequence of KATLTADKSSSTAYMQLSSLTSEDSAVYYCAR (SEQ ID NO: 48); and

(d) an FR-H4 comprising the amino acid sequence of WGAGTTVTVS (SEQ ID NO: 49).

119. The method of any one of embodiments 116-118, wherein the anti-CD20 antibody further comprises:

(a) a VH domain comprising an amino acid sequence having at least 95% sequence identity to the amino acid sequence of

(SEQ ID NO: 50)
QVQLQQPGAELVKPGASVKMSCKASGYTFTSYNMHWVKQTPGRGLEWIGAI
YPGNGDTSYNQKFKGKATLTADKSSSTAYMQLSSLTSEDSAVYYCARSTYY
GGDWYFNVWGAGTTVTVS;

(b) a VL domain comprising an amino acid sequence having at least 95% sequence identity to the amino acid sequence of QIVLSQSPAILSASPGEKVTMTCRASSSVSYIHWFQQKPGSSPKPWIYATSNLASGVPVRFSGSGSG TSYSLTISRVEAEDAATYYCQQWTSNPPTFGGGTKLEIK (SEQ ID NO: 51); or

(c) a VH domain as in (a) and a VL domain as in (b).

120. The method of embodiment 119, wherein the anti-CD20 antibody comprises:

(a) a VH domain comprising the amino acid sequence of SEQ ID NO:50; and

(b) a VL domain comprising the amino acid sequence of SEQ ID NO:51.

121. The method of any one of embodiments 96-120, wherein the anti-CD20 antibody is a monoclonal antibody.

122. The method of any one of embodiments 96-120, wherein the anti-CD20 antibody is a chimeric antibody.

123. The method of any one of embodiments 96-122, wherein the anti-CD20 antibody is a full-length antibody.

124. The method of any one of embodiments 96-123, wherein the anti-CD20 antibody is rituximab.

125. The method of any one of embodiments 96-122, wherein the anti-CD20 antibody is an antibody fragment that binds CD20 selected from the group consisting of Fab, Fab′, Fab′-SH, Fv, single chain variable fragment (scFv), and (Fab′)₂ fragments.

126. The method of any one of embodiments 96-125, wherein the anti-CD20 antibody is an IgG class antibody.

127. The method of embodiment 126, wherein the IgG class antibody is an IgG1 subclass antibody.

128. The method of any one of embodiments 96-127, wherein the method comprises administering to the subject the anti-CD20 antibody intravenously.

129. The method of any one of embodiments 96-128, wherein the hematologic cancer is a lymphoma.

130. The method of embodiment 129, wherein the lymphoma is a non-Hodgkin's lymphoma (NHL).

131. The method of embodiment 130, wherein the NHL is a relapsed or refractory diffuse large B cell lymphoma (DLBCL).

132. The method of embodiment 130, wherein the NHL is a relapsed or refractory follicular lymphoma (FL).

133. The method of any one of embodiments 1-132, wherein the method comprises administering to the subject an anti-TIGIT antagonist antibody at a fixed dose of between about 30 mg to about 600 mg.

134. The method of embodiment 133, wherein the method comprises administering to the subject an anti-TIGIT antagonist antibody at a fixed dose of about 600 mg.

135. The method of any one of embodiments 1-134, wherein the anti-TIGIT antagonist antibody comprises the following CDRs:

(a) a CDR-H1 comprising the amino acid sequence of SNSAAWN (SEQ ID NO: 1);

(b) a CDR-H2 comprising the amino acid sequence of KTYYRFKWYSDYAVSVKG (SEQ ID NO: 2);

(c) a CDR-H3 comprising the amino acid sequence of ESTTYDLLAGPFDY (SEQ ID NO: 3);

(d) a CDR-L1 comprising the amino acid sequence of KSSQTVLYSSNNKKYLA (SEQ ID NO: 4);

(e) a CDR-L2 comprising the amino acid sequence of WASTRES (SEQ ID NO: 5); and

(f) a CDR-L3 comprising the amino acid sequence of QQYYSTPFT (SEQ ID NO: 6).

136. The method of embodiment 1-135, wherein the anti-TIGIT antagonist antibody further comprises the following light chain variable region FRs:

(a) an FR-L1 comprising the amino acid sequence of DIVMTQSPDSLAVSLGERATINC (SEQ ID NO: 7);

(b) an FR-L2 comprising the amino acid sequence of WYQQKPGQPPNLLIY (SEQ ID NO: 8);

(c) an FR-L3 comprising the amino acid sequence of GVPDRFSGSGSGTDFTLTISSLQAEDVAVYYC (SEQ ID NO: 9); and

(d) an FR-L4 comprising the amino acid sequence of FGPGTKVEIK (SEQ ID NO: 10).

137. The method of embodiment 136, wherein the anti-TIGIT antagonist antibody further comprises the following heavy chain variable region FRs:

(a) an FR-H1 comprising the amino acid sequence of X₁VQLQQSGPGLVKPSQTLSLTCAISGDSVS (SEQ ID NO: 11), wherein X1 is Q or E;

(b) an FR-H2 comprising the amino acid sequence of WIRQSPSRGLEWLG (SEQ ID NO: 12);

(c) an FR-H3 comprising the amino acid sequence of RITINPDTSKNQFSLQLNSVTPEDTAVFYCTR (SEQ ID NO: 13); and

(d) an FR-H4 comprising the amino acid sequence of WGQGTLVTVSS (SEQ ID NO: 14).

138. The method of embodiment 137, wherein X1 is Q.

139. The method of embodiment 137, wherein X1 is E.

140. The method of any one of embodiments 135-139, wherein the anti-TIGIT antagonist antibody comprises:

(a) a VH domain comprising an amino acid sequence having at least 95% sequence identity to the amino acid sequence of

(SEQ ID NO: 17)
EVQLQQSGPGLVKPSQTLSLTCAISGDSVSSNSAAWNWIRQSPSRGLEWLG
KTYYRFKWYSDYAVSVKGRITINPDTSKNQFSLQLNSVTPEDTAVFYCTRE
STTYDLLAGPFDYWGQGTLVTVSS
or
(SEQ ID NO: 18)
QVQLQQSGPGLVKPSQTLSLTCAISGDSVSSNSAAWNWIRQSPSRGLEWLG
KTYYRFKWYSDYAVSVKGRITINPDTSKNQFSLQLNSVTPEDTAVFYCTRE
STTYDLLAGPFDYWGQGTLVTVSS;

(b) a VL domain comprising an amino acid sequence having at least 95% sequence identity to the

amino acid sequence of DIVMTQSPDSLAVSLGERATINCKSSQTVLYSSNNKKYLAWYQQKPGQPPNLLIYWASTRESGVPDR FSGSGSGTDFTLTISSLQAEDVAVYYCQQYYSTPFTFGPGTKVEIK (SEQ ID NO: 19); or

(c) a VH domain as in (a) and a VL domain as in (b).

141. The method of embodiment 140, wherein the anti-TIGIT antagonist antibody comprises:

(a) a VH domain comprising the amino acid sequence of SEQ ID NO: 17 or 18; and

(b) a VL domain comprising the amino acid sequence of SEQ ID NO: 19.

142. The method of any one of embodiments 1-141, wherein the anti-TIGIT antagonist antibody is a monoclonal antibody.

143. The method of any one of embodiments 1-142, wherein the anti-TIGIT antagonist antibody is a human antibody.

144. The method of any one of embodiments 1-143, wherein the anti-TIGIT antagonist antibody is a full-length antibody.

145. The method of any one of embodiments 1-137 and 139-144, wherein the anti-TIGIT antagonist antibody is tiragolumab.

146. The method of any one of embodiments 1-143, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

147. The method of any one of claims 1-146, wherein the anti-TIGIT antagonist antibody is an IgG class antibody.

148. The method of embodiment 147, wherein the IgG class antibody is an IgG1 subclass antibody.

149. The method of any one of embodiments 1-148, wherein the anti-TIGIT antagonist antibody is administered intravenously.

150. A method for treating a subject having a hematologic cancer, the method comprising administering to the subject an effective amount of tiragolumab and an effective amount of daratumumab.

151. The method of embodiment 150, wherein the hematological cancer is myeloma.

152. The method of embodiment 151, wherein the myeloma is MM.

153. The method of embodiment 152, wherein the MM is relapsed or refractory MM.

154. The method of any one of embodiments 150-153, wherein the tiragolumab is administered at a fixed dose of 600 mg.

155. The method of any one of embodiments 150-154, wherein the daratumumab is administered at a dose of 16 mg/kg.

156. A method of treating a subject having a hematologic cancer, the method comprising administering to the subject an effective amount of tiragolumab and an effective amount of rituximab.

157. The method of embodiment 156, wherein the hematologic cancer is lymphoma.

158. The method of embodiment 157, wherein the lymphoma is NHL.

159. The method of embodiment 158, wherein the NHL is relapsed or refractory NHL.

160. The method of any one of embodiments 156-159, wherein the tiragolumab is administered at a fixed dose of 600 mg.

161. The method of any one of embodiments 156-160, wherein the rituximab is administered at a dose of 375 mg/m².

162. The method of any one of embodiments 156-161, wherein the rituximab is administered in a total of eight doses.

163. A method for treating a subject having a relapsed or refractory MM, the method comprising administering to the subject tiragolumab at a fixed dose of 600 mg and daratumumab at a dose of 16 mg/kg in a dosing regimen comprising at least nine dosing cycles, wherein the length of each dosing cycle is 21 days, and wherein:

(a) tiragolumab is administered on or about day 1 of each dosing cycle; and

(b) daratumumab is administered on or about days 1, 8, and 15 of each of dosing cycles 1-3, on or about day 1 during each of dosing cycles 4-8, and once every 4 weeks beginning on or about day 1 of dosing cycle 9.

164. A method of treating a subject having a relapsed or refractory NHL, the method comprising administering to the subject tiragolumab at a fixed dose of 600 mg and rituximab at a dose of 375 mg/m² in a dosing regimen comprising at least a first, a second, and a third dosing cycle, wherein the length of each dosing cycle is 21 days, and wherein:

(a) each dosing cycle comprises a single dose of tiragolumab administered on or about day 1 of each dosing cycle;

(b) the first dosing cycle comprises a first dose (C1D1), a second dose (C1D2), and a third dose (C1D3) of rituximab, wherein the C1 D1, the C1 D2, and the C1 D3 are administered on or about days 1, 8, and 15, respectively, of the first dosing cycle;

(c) the second dosing cycle further comprises a first dose (C2D1), a second dose (C2D2), and a third dose (C2D3) of rituximab, wherein the C2D1, the C2D2, and the C2D3 are administered on or about days 1, 8, and 15, respectively, of the second dosing cycle; and

(d) the third dosing cycle further comprises a first dose (C3D1) and a second dose (C3D2) of rituximab, wherein the C3D1 and the C3D2 are administered on or about days 1 and 8, respectively, of the third dosing cycle, and

wherein the dosing regimen comprises a total of eight doses of rituximab.

165. The method of embodiment 163 or 164, wherein the dosing regimen comprises at least 12 dosing cycles.

166. The method of embodiment 165, wherein the dosing regimen comprises at least 16 dosing cycles.

167. A kit comprising an anti-TIGIT antagonist antibody, an anti-CD38 antibody, and a package insert comprising instructions to administer the anti-TIGIT antagonist antibody and the anti-CD38 antibody to a subject having a hematologic cancer in accordance with the methods of any one of embodiments 1-31, 59-95, and 133-149.

168. The kit of embodiment 167, wherein the anti-TIGIT antagonist antibody is tigarolumab and the anti-CD38 antibody is daratumumab.

169. A kit comprising an anti-TIGIT antagonist antibody, an anti-CD20 antibody, and a package insert comprising instructions to administer the anti-TIGIT antagonist antibody and the anti-CD20 antibody to a subject having a hematologic cancer in accordance with the methods of any one of embodiments 32-58 and 96-149.

170. The kit of embodiment 169, wherein the anti-TIGIT antagonist antibody is tiragolumab and the anti-CD20 antibody is rituximab.

171. A method for treating a subject having a relapsed or refractory MM, the method comprising administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle.

172. A method for treating a subject having a relapsed or refractory NHL, the method comprising administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle.

173. The method of embodiment 171 or 172, wherein tiragolumab is administered as a monotherapy.

174. The method of any one of embodiments 171-173, wherein the method comprises an observation period following administration of tiragolumab.

175. The method of embodiment 174, wherein the observation period is between about 30 minutes to about 60 minutes in length.

176. The method of any one of embodiments 171-175, wherein the dosing regimen comprises at least 12 dosing cycles.

177. The method of embodiment 176, wherein the dosing regimen comprises at least 16 dosing cycles.

178. The method of any one of embodiments 171-177, wherein the subject has an infusion-related reaction (IRR) to tiragolumab, and the method further comprises administering to the subject an antihistamine and/or an antipyretic prior to a subsequent administration of tiragolumab.

179. The method of any one of embodiments 171-178, wherein the method comprises administering to the subject tiragolumab intravenously.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating a subject having a hematologic cancer, the method comprising administering to the subject an anti-TIGIT antagonist antibody at a fixed dose of between about 30 mg to about 1200 mg and an anti-CD38 antibody at a dose of between about 8 mg/kg to about 24 mg/kg in a dosing regimen comprising at least nine dosing cycles, wherein:
(a) the anti-TIGIT antagonist antibody is administered once every three weeks; and
(b) the anti-CD38 antibody is administered once every week during each of dosing cycles 1-3, once every three weeks during each of dosing cycles 4-8, and once every four weeks beginning on dosing cycle 9.

2. The method of claim 1, wherein the length of each dosing cycle is 21 days.

3. The method of claim 1, wherein:
(a) the anti-TIGIT antagonist antibody is administered on or about day 1 of each dosing cycle; and/or
(b) the anti-CD38 antibody is administered on or about days 1, 8, and 15 of each of dosing cycles 1-3, on or about day 1 of each of dosing cycles 4-8, and on or about day 1 dosing cycle 9.

4. (canceled)

5. The method of claim 1, wherein the anti-TIGIT antagonist antibody and the anti-CD38 antibody are both administered on or about day 1 of each of dosing cycles 1-9 and the anti-TIGIT antagonist antibody is administered prior to the anti-CD38 antibody.

6-11. (canceled)

12. The method of claim 1, wherein the dosing regimen comprises at least 12 dosing cycles or at least 16 dosing cycles.

13-16. (canceled)

17. The method of claim 1, further comprising:
(a) administering to the subject a corticosteroid, an antipyretic, and/or an antihistamine prior to each administration of the anti-CD38 antibody; and/or
(b) administering to the subject a corticosteroid on each of the two days following administration of the anti-CD38 antibody.

18-19. (canceled)

20. The method of claim 1, wherein the method comprises administering to the subject the anti-CD38 antibody at a dose of about 16 mg/kg.

21. The method of claim 1, wherein the anti-CD38 antibody is an anti-CD38 antagonist antibody.

22-29. (canceled)

30. The method of claim 1, wherein the anti-CD38 antibody is daratumumab.

31-33. (canceled)

34. The method of claim 1, wherein the method comprises administering to the subject the anti-CD38 antibody intravenously.

35. The method of claim 1, wherein the hematologic cancer is a myeloma, a multiple myeloma (MM), or a relapsed or refractory MM.

36-37. (canceled)

38. A method for treating a subject having a hematologic cancer, the method comprising administering to the subject an anti-TIGIT antagonist antibody at a fixed dose of between about 30 mg to about 1200 mg and an anti-CD20 antibody at a dose of between about 250 mg/m² to about 500 mg/m² in a dosing regimen comprising at least a first, a second, and a third dosing cycle, wherein:
(a) the anti-TIGIT antagonist antibody is administered once every three weeks; and
(b) the anti-CD20 antibody is administered once every week.

39. The method of claim 38, wherein:
(a) each dosing cycle of the dosing regimen comprises a single dose of the anti-TIGIT antagonist antibody;
(b) the first dosing cycle comprises a first dose (C1D1), a second dose (C1D2), and a third dose (C1D3) of the anti-CD20 antibody;
(c) the second dosing cycle comprises a first dose (C2D1), a second dose (C2D2), and a third dose (C2D3) of the anti-CD20 antibody; and
(d) the third dosing cycle comprises at least a first dose (C3D1) and a second dose (C3D2) of the anti-CD20 antibody.

40. The method of claim 38, wherein the dosing regimen comprises a total of eight doses of the anti-CD20 antibody.

41. The method of claim 38, wherein the length of each dosing cycle is 21 days.

42. The method of claim 39, wherein the method comprises:
(a) administering to the subject the anti-TIGIT antagonist antibody on or about day 1 of each dosing cycle;
(b) administering to the subject the C1D1, the C1D2, and the C1D3 of the anti-CD20 antibody on or about days 1, 8, and 15, respectively, of the first dosing cycle;
(c) administering to the subject the C2D1, the C2D2, and the C2D3 of the anti-CD20 antibody on or about days 1, 8, and 15, respectively, of the second dosing cycle; and/or
(d) administering to the subject the C3D1 and the C3D2 of the anti-CD20 antibody on or about days 1 and 8, respectively, of the third dosing cycle.

43-45. (canceled)

46. The method of claim 38, wherein the anti-TIGIT antagonist antibody and the anti-CD20 antibody are both administered on or about day 1 of each of dosing cycles 1, 2, and 3, and the anti-TIGIT antagonist antibody is administered prior to the anti-CD20 antibody.

47-49. (canceled)

50. The method of claim 38, wherein the dosing regimen comprises at least 12 dosing cycles or at least 16 dosing cycles.

51. (canceled)

52. The method of claim 38, wherein the subject has an infusion-related reaction (IRR) to the anti-TIGIT antagonist antibody, and the method further comprises administering to the subject an antihistamine and/or an antipyretic prior to a subsequent administration of the anti-TIGIT antagonist antibody.

53. The method of claim 38, further comprising administering to the subject an antipyretic and an antihistamine and/or a glucocorticoid prior to each administration of the anti-CD20 antibody.

54-55. (canceled)

56. The method of claim 38, wherein the method comprises administering to the subject the anti-CD20 antibody at a dose of about 375 mg/m².

57. The method of claim 38, wherein the anti-CD20 antibody is an anti-CD20 antagonist antibody.

58-65. (canceled)

66. The method of claim 38, wherein the anti-CD20 antibody is rituximab.

67-69. (canceled)

70. The method of claim 38, wherein the method comprises administering to the subject the anti-CD20 antibody intravenously.

71. The method of claim 38, wherein the hematologic cancer is a lymphoma, a non-Hodgkin's lymphoma (NHL), is a relapsed or refractory diffuse large B cell lymphoma (DLBCL), or a relapsed or refractory follicular lymphoma (FL).

72-74. (canceled)

75. The method of claim 1, wherein the method comprises administering to the subject the anti-TIGIT antagonist antibody at a fixed dose of between about 30 mg to about 600 mg.

76. The method of claim 75, wherein the method comprises administering to the subject the anti-TIGIT antagonist antibody at a fixed dose of about 600 mg.

77-86. (canceled)

87. The method of claim 1, wherein the anti-TIGIT antagonist antibody is tiragolumab.

88-90. (canceled)

91. The method of claim 1, wherein the anti-TIGIT antagonist antibody is administered intravenously.

92-95. (canceled)

96. A kit comprising an anti-TIGIT antagonist antibody, an anti-CD38 antibody, and a package insert comprising instructions to administer the anti-TIGIT antagonist antibody and the anti-CD38 antibody to a subject having a hematologic cancer in accordance with the method of claim 1.

97. The kit of claim 96, wherein the anti-TIGIT antagonist antibody is tiragolumab and the anti-CD38 antibody is daratumumab.

98. A kit comprising an anti-TIGIT antagonist antibody, an anti-CD20 antibody, and a package insert comprising instructions to administer the anti-TIGIT antagonist antibody and the anti-CD20 antibody to a subject having a hematologic cancer in accordance with the method of claim 38.

99. The kit of claim 98, wherein the anti-TIGIT antagonist antibody is tiragolumab and the anti-CD20 antibody is rituximab.

100. A method for treating a subject having a relapsed or refractory MM, the method comprising administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle.

101. A method for treating a subject having a relapsed or refractory NHL, the method comprising administering to the subject tiragolumab at a fixed dose of 600 mg in a dosing regimen comprising one or more dosing cycles, wherein the length of each dosing cycle is 21 days, and wherein tiragolumab is administered on or about day 1 of each dosing cycle.

102. The method of claim 100, wherein tiragolumab is administered as a monotherapy.

103-104. (canceled)

105. The method of claim 100, wherein the dosing regimen comprises at least 12 dosing cycles or at least 16 dosing cycles.

106. (canceled)

107. The method of claim 100, wherein the subject has an infusion-related reaction (IRR) to tiragolumab, and the method further comprises administering to the subject an antihistamine and/or an antipyretic prior to a subsequent administration of tiragolumab.

108. The method of claim 100, wherein the method comprises administering to the subject tiragolumab intravenously.

109. The method of claim 38, wherein the method comprises administering to the subject the anti-TIGIT antagonist antibody at a fixed dose of between about 30 mg to about 600 mg.

110. The method of claim 109, wherein the method comprises administering to the subject the anti-TIGIT antagonist antibody at a fixed dose of about 600 mg.

111. The method of claim 38, wherein the anti-TIGIT antagonist antibody is tiragolumab.

112. The method of claim 38, wherein the anti-TIGIT antagonist antibody is administered intravenously.

113. The method of claim 101, wherein tiragolumab is administered as a monotherapy.

114. The method of claim 101, wherein the dosing regimen comprises at least 12 dosing cycles or at least 16 dosing cycles.

115. The method of claim 101, wherein the subject has an infusion-related reaction (IRR) to tiragolumab, and the method further comprises administering to the subject an antihistamine and/or an antipyretic prior to a subsequent administration of tiragolumab.

116. The method of claim 101, wherein the method comprises administering to the subject tiragolumab intravenously.


SEQUENCE LISTING (57 sequences)

SEQ ID NO: 1 - protein, 7 aa, Homo sapiens
SNSAAWN

SEQ ID NO: 2 - protein, 18 aa, Homo sapiens
KTYYRFKWYSDYAVSVKG

SEQ ID NO: 3 - protein, 14 aa, Homo sapiens
ESTTYDLLAGPFDY

SEQ ID NO: 4 - protein, 17 aa, Homo sapiens
KSSQTVLYSSNNKKYLA

SEQ ID NO: 5 - protein, 7 aa, Homo sapiens
WASTRES

SEQ ID NO: 6 - protein, 9 aa, Homo sapiens
QQYYSTPFT

SEQ ID NO: 7 - protein, 23 aa, Homo sapiens
DIVMTQSPDSLAVSLGERATINC

SEQ ID NO: 8 - protein, 15 aa, Homo sapiens
WYQQKPGQPPNLLIY

SEQ ID NO: 9 - protein, 32 aa, Homo sapiens
GVPDRFSGSGSGTDFTLTISSLQAEDVAVYYC

SEQ ID NO: 10 - protein, 10 aa, Homo sapiens
FGPGTKVEIK

SEQ ID NO: 11 - protein, 30 aa, Homo sapiens
  MISC_FEATURE (1)..(1): Xaa is Gln or Glu
XVQLQQSGPGLVKPSQTLSLTCAISGDSVS

SEQ ID NO: 12 - protein, 14 aa, Homo sapiens
WIRQSPSRGLEWLG

SEQ ID NO: 13 - protein, 32 aa, Homo sapiens
RITINPDTSKNQFSLQLNSVTPEDTAVFYCTR

SEQ ID NO: 14 - protein, 11 aa, Homo sapiens
WGQGTLVTVSS

SEQ ID NO: 15 - protein, 30 aa, Homo sapiens
EVQLQQSGPGLVKPSQTLSLTCAISGDSVS

SEQ ID NO: 16 - protein, 30 aa, Homo sapiens
QVQLQQSGPGLVKPSQTLSLTCAISGDSVS

SEQ ID NO: 17 - protein, 126 aa, Homo sapiens
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 18 - protein, 126 aa, Homo sapiens
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 19 - protein, 113 aa, Homo sapiens
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 20 - protein, 5 aa, Homo sapiens
SFAMS

SEQ ID NO: 21 - protein, 17 aa, Homo sapiens
AISGSGGGTYYADSVKG

SEQ ID NO: 22 - protein, 13 aa, Homo sapiens
DKILWFGEPVFDY

SEQ ID NO: 23 - protein, 11 aa, Homo sapiens
RASQSVSSYLA

SEQ ID NO: 24 - protein, 7 aa, Homo sapiens
DASNRAT

SEQ ID NO: 25 - protein, 10 aa, Homo sapiens
QQRSNWPPTF

SEQ ID NO: 26 - protein, 23 aa, Homo sapiens
EIVLTQSPATLSLSPGERATLSC

SEQ ID NO: 27 - protein, 15 aa, Homo sapiens
WYQQKPGQAPRLLIY

SEQ ID NO: 28 - protein, 32 aa, Homo sapiens
GIPARFSGSGSGTDFTLTISSLEPEDFAVYYC

SEQ ID NO: 29 - protein, 9 aa, Homo sapiens
GQGTKVEIK

SEQ ID NO: 30 - protein, 30 aa, Homo sapiens
EVQLLESGGGLVQPGGSLRLSCAVSGFTFN

SEQ ID NO: 31 - protein, 14 aa, Homo sapiens
WVRQAPGKGLEWVS

SEQ ID NO: 32 - protein, 32 aa, Homo sapiens
RFTISRDNSKNTLYLQMNSLRAEDTAVYFCAK

SEQ ID NO: 33 - protein, 11 aa, Homo sapiens
WGQGTLVTVSS

SEQ ID NO: 34 - protein, 122 aa, Homo sapiens [sequence omitted]

SEQ ID NO: 35 - protein, 107 aa, Homo sapiens [sequence omitted]

SEQ ID NO: 36 - protein, 5 aa, Artificial Sequence
  Synthetic Construct
SYNMH

SEQ ID NO: 37 - protein, 17 aa, Artificial Sequence
  Synthetic Construct
AIYPGNGDTSYNQKFKG

SEQ ID NO: 38 - protein, 12 aa, Artificial Sequence
  Synthetic Construct
STYYGGDWYFNV

SEQ ID NO: 39 - protein, 10 aa, Artificial Sequence
  Synthetic Construct
RASSSVSYIH

SEQ ID NO: 40 - protein, 7 aa, Artificial Sequence
  Synthetic Construct
ATSNLAS

SEQ ID NO: 41 - protein, 9 aa, Artificial Sequence
  Synthetic Construct
QQWTSNPPT

SEQ ID NO: 42 - protein, 23 aa, Artificial Sequence
  Synthetic Construct
QIVLSQSPAILSASPGEKVTMTC

SEQ ID NO: 43 - protein, 15 aa, Artificial Sequence
  Synthetic Construct
WFQQKPGSSPKPWIY

SEQ ID NO: 44 - protein, 32 aa, Artificial Sequence
  Synthetic Construct
GVPVRFSGSGSGTSYSLTISRVEAEDAATYYC

SEQ ID NO: 45 - protein, 10 aa, Artificial Sequence
  Synthetic Construct
FGGGTKLEIK

SEQ ID NO: 46 - protein, 30 aa, Artificial Sequence
  Synthetic Construct
QVQLQQPGAELVKPGASVKMSCKASGYTFT

SEQ ID NO: 47 - protein, 14 aa, Artificial Sequence
  Synthetic Construct
WVKQTPGRGLEWIG

SEQ ID NO: 48 - protein, 32 aa, Artificial Sequence
  Synthetic Construct
KATLTADKSSSTAYMQLSSLTSEDSAVYYCAR

SEQ ID NO: 49 - protein, 10 aa, Artificial Sequence
  Synthetic Construct
WGAGTTVTVS

SEQ ID NO: 50 - protein, 120 aa, Artificial Sequence
  Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVS

SEQ ID NO: 51 - protein, 106 aa, Artificial Sequence [sequence omitted]

SEQ ID NO: 52 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 53 - protein, 223 aa, Homo sapiens [sequence omitted]

SEQ ID NO: 54 - DNA, 3594 bp, Homo sapiens
gtctatcagcgatttcatcttcaggcctggactacaccactcaccctcccagtgtgcttgagaaacaaactgcacccactgaactccgcagctagcatccaaatcagcccttgagatttgaggccttggagactcagatcctgaacaagagagaacaaaatctctactttgatggaacttccattctgtggggaagagactgacaataagcaattaaataaataagaactcagcagtaggccttgcctcagatccaaggtcactcggaagaggccatgtctaccctcaatgacactcatggaggaaatgctgagagaagcattcagatgcatgacacaaggtaagactgccaaaaatcttgttcttgctctcctcattttgttatttgttttatttttaggagttttgagagcaaaatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtatttttttttgtcattttctccatcaacaaccagggagactgcacctgatggaaaagatatatgactgcttcatgacattcctaaactatcttttttttattccacatctacgtttttggtggagtcccttttgcatcattgttttaaggatgataaaaaaaaataacaactagggacaatacagaacccattccatttatctttctacagggctgacattgtggcacattcttagagttaccacaccccatgagggaagctctaaatagccaacacccatctgttttttgtaaaaacagcatagcttatacatggacatgtctctgccttaacttttcctaactcccactctaggctattgtttgcatgtctacctacttttagccattatgcgagaaaagaaaaaaatgaccatagaaaatgccaccatgaggtgcccaaatttcaaataataattaacatttagttatatttataatttccagatgacaaagtatttcatcaaataacttcatttgatgttccatgatcaagaaagaatccctatctctattttacaagtaattcaaagaggccaaataacttgtaaacaagaaaaggtaacttgtcaacagtcataactagtaattatgagagccttgtttcataaccaggtcttcttactcaaatcctgtgatgtttgaaataaccaaattgtctctccaatgtctgcataaactgtgagagccaagtcaacagcttttatcaagaatttactctctgaccagcaataaacaagcactgagagacacagagagccagattcagattttacccatggggataaaaagactcagactttcaccacatttggaaaactacttgcatcataaatatataataactggtagtttatatgaagcagacactaagtgctatagacactctcagaatatcatacttggaaacaatgtaattaaaatgccgaatctgagtcaacagctgccctacttttcaattcagatatactagtaccttacctagaaataatgttaacctagggtgaagtcactataatctgtagtctattatttgggcatttgctacatgatgagtgctgccagattgtggcaggtaaagagacaatgtaatttgcactccctatgatatttctacatttttagcgaccactagtggaagacattccccaaaattagaaaaaaaggagatagaagatttctgtctatgtaaagttctcaaaatttgttctaaattaataaaactatctttgtgttcttttctgcaacagatgattccaacatgggtgtttgtctattcttctttactcttgaaacattagaccatgggaggctcttacagccttgagttgatatttatacaacccaaatctaggtttgaacggtgaggtgtcaggtcatcaaatattcatgtctatatagtcttacacaggttctcaaaaaaaatgttcatgggataggtcattgataatggattccttattctgagaactccagacgactgaaatatatgagagaaggaaaaggacatagtaggagcaggcctgagaaaaaaatgaaagtcagaaatctttaaaaaaatacaagatcttatttctatcttattttttctcctcttctgaaatatatatgaggattcctctccaaacccatggtttctctaagaattttgagtcatttgtatgacctcaaataattagttttagctgacctcacataactccttataataggagacatctttaatgtctgctattaaagaaggatgaaaattcctatgaccttctccccgattatccctttggcaatatagagtcaaataataacattgaccaatagtaaacatgctttgccaagaagtagaagatatattctctagccttagtttttcctcccaatttgcatttttgtaaaaataatgttgtatccacaaaggaaataaactttaaaaacccaagtgca

SEQ ID NO: 55 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 56 - DNA, 5620 bp, Homo sapiens
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 57 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI